SEQUENCE LISTING (9 sequences)

SEQ ID NO: 1 - protein, 219 aa, Artificial Sequence
  Synthetic Construct
DIVMTQSPDSLAVSLGERATINCKSSQSLLNSGTRKNYLAWYQQKPGQPPKLLISWASSRESGVPDRFSGSGSGTDFTLTISSLQAEDVAVYYCKQSYNLFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 389 aa, Artificial Sequence
  Synthetic Construct
SALMSRLTISKDTSKNQFSLKLSSVTAADTAVYYCARDENWDGAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQAYICNVNHKPSNTKVDKKVGPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIERTISKAKGQPREPQVYTLPPSRDELAKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 17 aa, Artificial Sequence
  Synthetic Construct
KSSQSLLNSGTRKNYLA

SEQ ID NO: 4 - protein, 7 aa, Artificial Sequence
  Synthetic Construct
WASSRES

SEQ ID NO: 5 - protein, 8 aa, Artificial Sequence
  Synthetic Construct
KQSYNLFT

SEQ ID NO: 6 - protein, 5 aa, Artificial Sequence
  Synthetic Construct
SYGVN

SEQ ID NO: 7 - protein, 16 aa, Artificial Sequence
  Synthetic Construct
VMWAGGSTNYNSALMS

SEQ ID NO: 8 - protein, 11 aa, Artificial Sequence
  Synthetic Construct
DENWDGAWFAY

SEQ ID NO: 9 - protein, 4 aa, Artificial Sequence
  Synthetic Construct
GPLG

(19) United States
(12) Patent Application Publication
(10) Pub. No.: US 2023/0256114 A1
(43) Pub. Date: Aug. 17, 2023

(54) NOVEL MAYTANSINOIDS AS ADC PAYLOADS AND THEIR USE FOR THE TREATMENT OF CANCER

(71) Applicant: Bionecure Therapeutics, Inc., Germantown, MD (US)

(72) Inventors: Dingguo LIU, San Diego, CA (US); Haifeng BAO, Bethesda, MD (US); Wei YUAN, Ashburn, VA (US)

(21) Appl. No.: 18/015,037

(22) PCT Filed: Jul. 2, 2021
(86) PCT No.: PCT/US2021/040347

§ 371 Date: Jan. 6, 2023

Related U.S. Application Data
(60) Provisional application No. 63/048,879, filed on Jul. 7, 2020.

(51) Int. Cl. A61K 47/68; A61P 35/00

(52) U.S. Cl. CPC A61K 47/6851; A61K 47/6803; A61K 47/6889; A61P 35/00

(57) ABSTRACT

The present invention describes novel maytansinoid and ansamitosin derivatives and methods for preparing payloads thereof bearing a linker with a functional group for conjugation to cell binding agents to generate cytotoxic drug conjugates. The present invention further relates to antibody drug conjugates conjugated to tumor associated antigen (TAA) antibody, preparation methods, pharmaceutical compositions and uses thereof for the treatment of cancer. The maytansinoid drug linker derivative payloads show increased solubility with decreased aggregation rate compared SMCC-DM1. An antibody drug conjugate containing a maytansinoid drug linker derivative payload and an anti-T rop-2 antibody (e.g., anti-T rop-2-BI-P203) was more or equally potent against Trop-2 high expressing tumors as compared to anti-T rop-2-SMCC-DM 1 or anti-T rop-2-vc-MMAE ADCs, with less effect on low or no antigen expressing cells, suggesting an increased therapeutic window. Methods relating to the use of the novel ADCs to treat antigen positive cells in cancers and immunological disorders are also provided herein.

### CROSS-REFERENCE TO RELATED APPLICATION

This application claims priority to U.S. provisional application 63/048,879, filed on Jul. 7, 2020, the contents of which is incorporated by reference in its entirety for all purposes.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 227362000240SEQLIST.TXT, date recorded: Jul. 2, 2021, size: 7 KB).

### FIELD OF THE INVENTION

The present invention describes novel maytansinoid and ansamitosin derivatives and methods for preparing maytansinoid and ansamitosin derivative payloads bearing a linker with a functional group that can be used for conjugation to cell binding agents to generate cytotoxic drug conjugates. The present invention further relates to therapeutic use of these conjugates for treatment of cancer as the conjugates are targeted and selectively delivered to a specific tumor cell population. The present invention further relates to antibody drug conjugates made with maytansinoid and ansamitosin derivative payloads bearing a linker moiety conjugated to tumor associated antigen (TAA) binding agents, preparation methods, pharmaceutical compositions and uses thereof for the treatment of cancer.

### BACKGROUND OF THE INVENTION

Today, cancer remains a major cause of death worldwide despite the numerous advanced diagnostic and therapeutic methods that have been developed. The major barrier to successful treatment and prevention of cancer lies in the fact that many cancers still fail to respond to the current chemotherapeutic and immunotherapy intervention, and many individuals suffer a recurrence or death, even after aggressive therapy.

Cancer immunotherapy is enjoying a renaissance, and in the past few years the rapidly advancing field has produced several new methods of treating cancer. Numerous cancer immunotherapy strategies have been the focus of extensive research and clinical evaluation including, but not limited to, treatment using depleting antibodies to specific tumor antigens; treatment using antibody-drug conjugates; treatment using agonistic, antagonistic, or blocking antibodies to co-stimulatory or co-inhibitory molecules (immune checkpoints); treatment using bispecific T cell engaging antibodies (BITE®) such as blinatumomab; treatment involving administration of biological response modifiers such as IL-2, IL-12, IL-15, IL-21, GM-CSF IFN-α, IFN-β and IFN-γ; treatment using therapeutic vaccines such as sipuleucel-T; treatment using dendritic cell vaccines, or tumor antigen peptide vaccines; treatment using chimeric antigen receptor (CAR)-T cells; treatment using CAR-NK cells; treatment using tumor infiltrating lymphocytes (TILs); and treatment using adoptively transferred anti-tumor T cells (ex vivo expanded and/or TCR transgenic).

Antibody-drug conjugates (ADCs) combine the binding specificity of an antibody with the potency of drugs such as, for example, cytotoxic agents, anticancer and immunosuppressive drugs. The use of ADCs allows the target-specific delivery of drugs which, if administered as unconjugated drugs, may result in unacceptable levels of toxicity to normal cells. The mechanism of an ADC is to recognize and bind to specific antigen through the antibodies, trigger a series of reactions, and then enter the cytoplasm through the endocytosis, where the highly cytotoxic drug is dissociated from the antibody after the degradation by lysosomal enzymes to kill cancer cells. Compared with the traditional chemotherapy which causes damage to both cancer cells and normal tissues indiscriminately, targeting drug delivery can make the drug act on cancer cells directly and reduce the damage to normal cells.

The cytotoxic compounds used in antibody-drug conjugates inhibit various essential cellular targets, such as microtubules (maytansinoids, auristatins, taxanes: U.S. Pat. Nos. 5,208,020; 5,416,064; 6,333,410; 6,441,163; 6,340,701: 6,372,738; 6,436,931; 6,596,757: 7,276,497; 7,301,019; 7,303,749; 7,368,565; 7,473,796; 7,585,857; 7,598,290: 7,495,114; 7,601,354, U.S. Patent Application Nos. 20100092495, 20100129314, 20090274713, 20090076263, 20080171865) and DNA (calicheamicin, doxorubicin, CC-1065 analogues: U.S. Pat. Nos. 5,475,092: 5,585,499; 5,846,545; 6,534,660; 6,756,397; 6,630,579; 7,388,026; 7,655,660; 7,655,661).

Maytansinoids are potent antimitotic agents that interfere with the formation of microtubules through the inhibition of the assembly of tubulin (Remillard, et al. (1975) Science 189: 1002-1005). Although maytansinoids are 100 to 1000-fold more cytotoxic than conventional chemotherapeutic agents like methotrexate, daunorubicin, and vincristine (U.S. Pat. No. 3,896,111), maytansinoids failed in human clinical trials due to an inadequate therapeutic window was observed.

Due to its high cytotoxicity, maytansinoids bearing various acyl side chains on the N-methylalanyl moiety suitable for linking to cell binding agents have been disclosed (see for example U.S. Pat. Nos. 5,208,020; 5,416,064; 7,473,796; 7,368,565; 7,301,019; 7,276,497; 6,716,821; 6,441,163: U.S. Patent Application Nos. 20100129314: 20100092495; 20090274713; 20090076263; 20080171865; 20080171856; 20070270585; 20070269447; 20070264266; and 20060167245; Chari et al., Cancer Res., 52: 127-131 (1992); Liu et al., Proc. Natl. Acad. Sci., 93:8618-8623 (1996); and Widdison et al., J. Med. Chem., 49; 4392, 2006). Among these conjugates, the cell-binding agent is linked via a cleavable disulfide bonds to the maytansinoids such as DM1 (N-deacetyl N-(3-mercapto-1-oxopropyl)-maytansine, CAS Number: 139504-50-0, FIG. 2) or DM4 (N-deacetyl-N-(4-mercapto-4-methyl-1-oxopentyl)-maytansine, CAS Number: 796073-69-31).

There still exists a great need for novel and effective ADCs for use in the treatment or to prevent recurrence of cancers and/or immunological disorders.

### SUMMARY OF THE INVENTION

The present invention describes novel maytansinoid and ansamitosin derivatives and methods for preparing maytansinoid and ansamitosin derivative payloads bearing a linker with a functional group that can be used for conjugation to cell binding agents to generate cytotoxic drug conjugates.

In one aspect, the present invention relates to an antibody drug conjugate (ADC) comprising an antibody (e.g., a cell binding antibody) chemically linked to a derivatized maytansinol or maytansinol analog residue represented by the following formula (I):

[MayO-L-]ₓ-Ab (I)

wherein x is about 1 to about 10;
Ab is an antibody (e.g., a cell binding antibody) or antigen binding fragment thereof;
wherein MayO is a maytansinol or maytansinol analog;
L is a bivalent linker comprising a N-methylalanine moiety represented by the following formula:

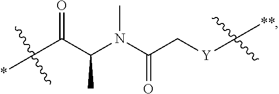

wherein * indicates the point of attachment to MayO and ** indicates the point of attachment to Ab; Y is selected from

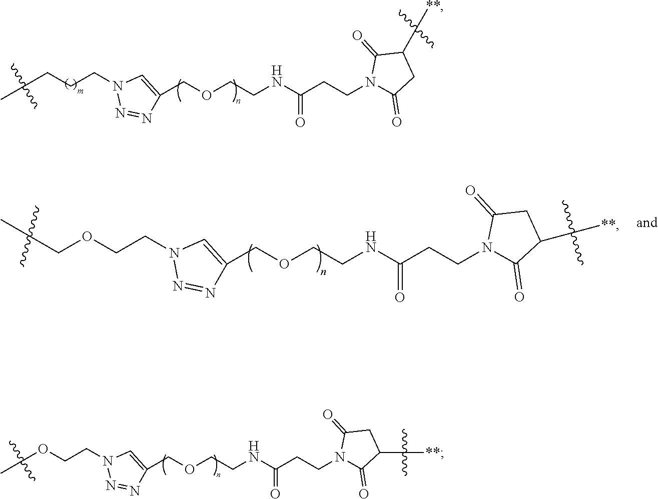

wherein m is 0-8, and n=2-12.

In various embodiments, Ab is an anti-Trop-2 antibody or an antigen binding fragment thereof.

In various embodiments, the invention relates to antibody drug conjugates wherein x is about 1 to about 10. In various embodiments, x is about 4 to about 7. In various embodiments, x is about 4. In various embodiments, x is about 6. In various embodiments, x is about 7.

In another aspect, the invention relates to derivatized maytansinol or maytansinol analogs represented by the following formula (II):

MayO-L′ (II)

wherein MayO is maytansinol or maytansinol analogs, L′ is a bivalent linker comprising a N-methylalanine moiety represented by the following formula:

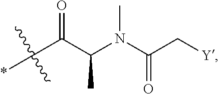

wherein * indicates the point of attachment to MayO; and Y′ comprises a functional group which can attach to an antibody (e.g., a cell binding antibody).

In various embodiments, the invention relates to derivatized maytansinol or maytansinol analogs wherein Y′ comprises pyrroline-dione.

In various embodiments, the invention relates to derivatized maytansinol or maytansinol analogs wherein Y′ is selected from

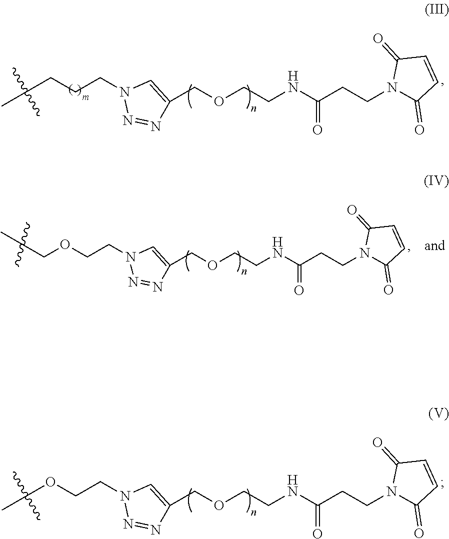

m is 0 to 8; and n is 2 to 12.

In various embodiments, Y′ is a linker reagent having the formula of (III):

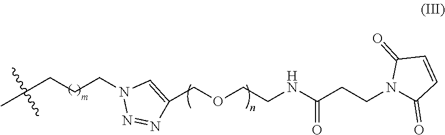

wherein m=0-8, and n=2-12.

In various embodiments, Y′ is a linker reagent having the formula of (IV)

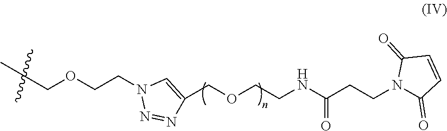

wherein n=2-12.

In various embodiments, Y′ is a linker reagent having the formula of (V)

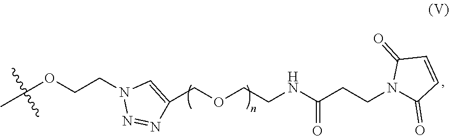

wherein n=2-12.

In another aspect, the invention relates to a derivatized maytansinol or maytansinol analog residue represented by the following formula (VI):

MayO-L²′ (VI)

wherein MayO is maytansinol or maytansinol analogs, L²′ is a bivalent linker represented by the following formula: *—C(═O)R—Y″, wherein
* indicates the point of attachment to MayO;
R is a 3-7 membered heterocyclyl, aryl or cyclic alkyl ring; and
Y″ comprises a functional group which can attach to an antibody (e.g., a cell binding antibody).

In another aspect, the invention relates to an antibody drug conjugate comprising an antibody (e.g., a cell binding antibody) chemically linked to a derivatized maytansinol or maytansinol analog residue represented by the following formula (VII):

[MayO-L²-]ₓ-Ab (VII)

wherein x is about 1 to about 10;
Ab is an antibody (e.g., a cell binding antibody) or an antigen binding fragment thereof; wherein MayO is maytansinol or a maytansinol analog;
L² is a bivalent linker represented by the following formula: *—C(═O)R—Y″—**, wherein
* indicates the point of attachment to MayO, ** indicates the point of attachment to Ab;
R is a 3-7 membered heterocyclyl, aryl or cyclic alkyl ring; and
Y″ comprises a functional group which can attach to a cell binding antibody.

In various embodiments, Ab is an anti-Trop-2 antibody or an antigen binding fragment thereof.

In various embodiments, the invention relates to an antibody drug conjugate where L² is a bivalent linker selected from:

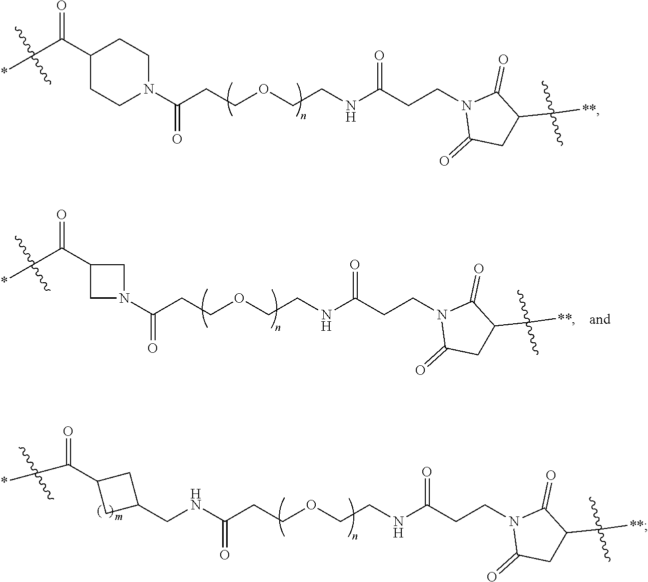

wherein m=0-3; and n=2-12.

In various embodiments, the invention relates to antibody drug conjugates wherein x is about 1 to about 10. In various embodiments, x is about 4 to about 7. In various embodiments, x is about 4. In various embodiments, x is about 6. In various embodiments, x is about 7.

In various embodiments, the invention relates to derivatized maytansinol or maytansinol analogs wherein L² comprises pyrroline-dione. In various embodiments of the invention, the heterocyclyl ring is selected from saturated or unsaturated 4-6 membered nitrogen containing heterocyclic rings. Examples of saturated heterocyclic radicals include saturated 3 to 6-membered heteromonocyclic group containing 1 to 4 nitrogen atoms [e.g. pyrrolidinyl, imidazolidinyl, piperidino, piperazinyl]; saturated 3 to 6-membered heteromonocyclic group containing 1 to 2 oxygen atoms and 1 to 3 nitrogen atoms [e.g. morpholinyl]; saturated 3 to 6-membered heteromonocyclic group containing 1 to 2 sulfur atoms and 1 to 3 nitrogen atoms [e.g., thiazolidinyl]. Examples of unsaturated heterocyclic radicals, also termed “heteroaryl” radicals, include unsaturated 5 to 6 membered heteromonocyclyl group containing 1 to 4 nitrogen atoms, for example, pyrrolyl, pyrrolinyl, imidazolyl, pyrazolyl, 2-pyridyl, 3-pyridyl, 4-pyridyl, pyrimidyl, pyrazinyl, pyridazinyl, triazolyl [e.g., 4H-1,2,4-triazolyl, 1H-1,2,3-triazolyl, 2H-1,2,3-triazolyl]; unsaturated condensed heterocyclic group containing 1 to 5 nitrogen atoms, for example, indolyl, isoindolyl, indolizinyl, benzimidazolyl, quinolyl, isoquinolyl, indazolyl, benzotriazolyl, tetrazolopyridazinyl [e.g., tetrazolo[1,5-b]pyridazinyl]; unsaturated 5- to 6-membered heteromonocyclic group containing 1 to 2 oxygen atoms and 1 to 3 nitrogen atoms, for example, oxazolyl, isoxazolyl, oxadiazolyl [e.g., 1,2,4-oxadiazolyl, 1,3,4-oxadiazolyl, 1,2,5-oxadiazolyl]; unsaturated condensed heterocyclic group containing 1 to 2 oxygen atoms and 1 to 3 nitrogen atoms [e.g. benzoxazolyl, benzoxadiazolyl]; and unsaturated 5 to 6-membered heteromonocyclic group containing 1 to 2 sulfur atoms and 1 to 3 nitrogen atoms, for example, thiazolyl, thiadiazolyl [e.g., 1,2,4-thiadiazolyl. In various embodiments of the invention, the cyclic alkyl ring, also know as a cycloalkyl ring, is a saturated cyclic alkyl group derived by the removal of one hydrogen atom from a single carbon atom of a parent cycloalkane. Typical cycloalkyl groups include, but are not limited to, groups derived from cyclopropane, cyclobutane, cyclopentane, cyclohexane, and cycloheptane, and the like.

In various embodiments, L²′ is a linker having the formula of (VIII):

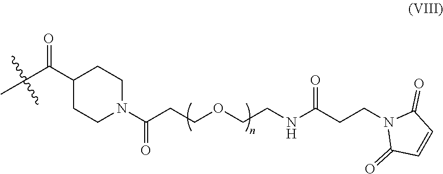

wherein n=2-12.

In various embodiments, L²′ is a linker having the formula of (IX):

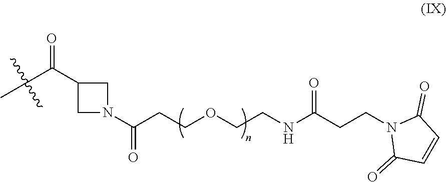

wherein n=2-12.

In various embodiments, L²′ is a linker having the formula of (X):

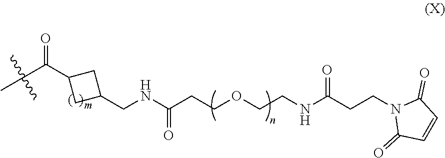

wherein m=0-3, and n=2-12.

The present invention provides an anti-Trop-2 antibody that is conjugated with maytansinol or maytansinol analogs, thus targeting disease cells or tissues. The anti-Trop-2 antibody binds to an antigen in the disease cells or tissues. A drug conjugated to the antibody exerts a cytotoxic, cytostatic, or immunosuppressive effect on the antigen-expressing cells to treat or prevent recurrence of Trop-2 positive cancers. The high affinity of the antibody drug conjugate ensures that the maytansinol or maytansinol analogs targets the tumor cells. The present technology provides a method to treat cancers by exerting cellular inhibitory or killing effect of maytansinol or maytansinol analogs on the Trop-2 positive cells.

In various embodiments, the ADC's comprise an L or L² that is a non-cleavable linker. In various embodiments, the ADC is an anti-Trop-2 antibody conjugated with maytansinol or maytansinol analogs. In various embodiments, the ADC is an anti-Trop-2 antibody conjugated with maytansinol or maytansinol analogs, wherein the maytansinol or maytansinol analogs is linked to an anti-Trop-2 antibody via a linker that is not acid labile. In various embodiments, the ADC is an anti-Trop-2 antibody conjugated with a maytansinol or maytansinol analogs, wherein the maytansinol or maytansinol analogs is linked to an anti-Trop-2 antibody via a linker that is not peptidase cathepsin sensitive. In various embodiments, the ADC is an anti-Trop-2 antibody conjugated with maytansinol or maytansinol analogs, wherein the maytansinol or maytansinol analogs is linked to an anti-Trop-2 antibody via a linker that does not contain a disulfide bond. In various embodiments, the ADC is an anti-Trop-2 antibody conjugated with maytansinol or maytansinol analogs, wherein the maytansinol or maytansinol analogs is linked to an anti-Trop-2 antibody via a linker that provides stability during circulation while being able to release the drug once inside the cells. Such linkers are contemplated to provide stability to the conjugated molecule prior to endocytosis, such as during circulation, to prevent premature degradation of the linker and release of the toxic drug, thus minimize the toxic effect of the drug.

In various embodiments there is provided one or more of a set of ADCs of Formulas I and VII wherein L and L² are cysteine reactive linkers.

In various embodiments there is provided one or more of a set of compounds of Formula II and VI wherein L′ and L²′ are non-cleavable linkers.

In various embodiments, the number of bonds formed between the drug-linker and cysteine residue on the anti-Trop-2 antibody is from 3 to 10. In various embodiments, the number of such bonds is at least 2, or alternatively at least 4, or 5. In various embodiments, the number of such formed bonds is no more than 10, or alternatively no more than 9, or 8, 7, 6, 5, or 4. In various embodiments, each anti-Trop-2 antibody, on average, is conjugated with about 4-7 drug molecules through cysteines.

The drug load on an anti-Trop-2 antibody may vary depending on many factors, such as the potency of the drug, the size, stability of the anti-Trop-2 antibody, conjugatable groups available on the anti-Trop-2 antibody, etc. In various embodiments, 1 to 10 maytansinol or maytansinol analogs molecules are conjugated with 1 anti-Trop-2 antibody molecule. In various embodiments, an average of about 4 to 7 maytansinol or maytansinol analogs drug molecules are conjugated with an anti-Trop-2 antibody molecule.

Another aspect of the invention relates to methods of inhibiting abnormal cell growth or treating a proliferative disorder, an autoimmune disorder, destructive bone disorder, infectious disease, viral disease, fibrotic disease, neurodegenerative disorder, pancreatitis or kidney disease in a mammal comprising administering to said mammal a therapeutically effective amount of the conjugate of formulas I and VII and, optionally, a chemotherapeutic agent.

Another aspect of the invention relates to pharmaceutical compositions of the cell binding agent conjugates of formulae I and VII and a pharmaceutically acceptable carrier, additive or diluent thereof

In various embodiments, the ADC constructs of the present invention comprise an Ab that is a targeting moiety, such as an antibody or antibody fragment capable of binding to a tumor associated antigen (TAA), a tissue-specific antigen, a cell surface molecule, extracellular matrix protein or protease(s), or any post-translational modification residue(s). In various embodiments, the ADC constructs of the present invention comprise an Ab that is a targeting moiety that exhibits binding affinity to a diseased cell or tissue.

In various embodiments, the antibody or antibody fragment is capable of binding to a TAA selected from the group consisting of: tumor-associated calcium signal transducer 2 (also known as Trop-2), Her2, Her3, Her4, EGF, EGFR, CD2, CD3, CD5, CD7, CD13, CD19, CD20, CD21, CD23, CD30, CD33, CD34, CD38, CD46, CD55, CD59, CD69, CD70, CD71, CD97, CD117, CD123, CD127, CD134, CD137, CD138, CD146, CD147, CD152, CD154, CD174, CD195, CD200, CD205, CD212, CD223, CD227, CD253, CD272, CD274, CD276, CD278, CD279, CD309, CD319, CD326, CD340, DR6, Kv1.3, 5E10, MUC1, uPA, MAGE3, MUC16, KLK3, K-ras, Mesothelin, p53, Survivin, G250, PSMA, Endoplasmin, BCMA, GPNMB, EphA2, EphB2, TMEFF2, Integrin beta 6, 5T4, CA9, IGF-1R, Axl, B7H3, B7H4, CDH6, HAVCR1, STEAP-1, STEAP-2, UPK2, CLDN18.

In various embodiments, the ADC comprises an TAA Ab selected from the group consisting of a fully human antibody, a humanized antibody, a chimeric antibody, a monoclonal antibody, a polyclonal antibody, a recombinant antibody, an antigen-binding antibody fragment, a Fab, a Fab′, a Fab₂, a Fab′₂, a IgG, a IgM, a IgA, a IgE, a scFv, a dsFv, a dAb, a nanobody, a unibody, and an diabody. In various embodiments, the antibody is a chimeric antibody. In various embodiments, the antibody is a humanized monoclonal antibody. In various embodiments, the antibody is a fully human monoclonal antibody.

In various embodiments the antibody is a humanized anti-Trop-2 antibody which comprises a light chain having an amino acid sequence as set forth in SEQ ID NO: 1 and a heavy chain having an amino acid sequence as set forth in SEQ ID NO: 2. In various embodiments the antibody is a humanized anti-Trop-2 antibody which comprises a heavy-chain variable domain having three complementary regions consisting of HCDR1 (SEQ ID NO: 6), HCDR2 (SEQ ID NO: 7), and HCDR3 (SEQ ID NO: 8), and a light-chain variable domain having three complementary regions consisting of LCDR1 (SEQ ID NO: 3), LCDR2 (SEQ ID NO: 4), and LCDR3 (SEQ ID NO: 5).

In another aspect, the present invention provides a pharmaceutical composition comprising the isolated ADC constructs in admixture with a pharmaceutically acceptable carrier.

In another aspect, the invention provides uses of the ADC constructs for the preparation of a medicament for the treatment of cancer.

In another aspect, the present invention provides a method for treating cancer or cancer metastasis in a subject, comprising administering a therapeutically effective amount of the pharmaceutical compositions of the invention to a subject in need thereof. In one embodiment, the subject is a human subject. In various embodiments, the cancer is selected from pancreatic cancer, gastric cancer, liver cancer, breast cancer, ovarian cancer, colorectal cancer, melanoma, leukemia, myelodysplastic syndrome, lung cancer, prostate cancer, brain cancer, bladder cancer, head-neck cancer, or rhabdomyosarcoma or any cancer.

In various embodiments, the subject previously responded to treatment with an anti-cancer therapy, but, upon cessation of therapy, suffered relapse (hereinafter “a recurrent cancer”). In various embodiments, the subject has a resistant or refractory cancer.

In another aspect, the present invention provides a method for treating cancer or cancer metastasis in a subject, comprising administering a therapeutically effective amount of the pharmaceutical compositions of the invention in combination with a second therapy selected from the group consisting of: cytotoxic chemotherapy, immunotherapy, small molecule kinase inhibitor targeted therapy, surgery, radiation therapy, stem cell transplantation, cell therapies including CAR-T, CAR-N K, iPS induced CAR-T or iPS induced CAR-NK and vaccine such as Bacille Calmette-Guerine (BCG). In various embodiments, the combination therapy may comprise administering to the subject a therapeutically effective amount of immunotherapy, including, but are not limited to, treatment using depleting antibodies to specific tumor antigens; treatment using antibody-drug conjugates; treatment using agonistic, antagonistic, or blocking antibodies to co-stimulatory or co-inhibitory molecules (immune checkpoints) such as CTLA-4, PD-1, PD-L1, CD40, OX-40, CD137, GITR, LAG3, TIM-3, Siglec 7, Siglec 8, Siglec 9, Siglec 15 and VISTA; treatment using bispecific T cell engaging antibodies (BITE®) such as blinatumomab: treatment involving administration of biological response modifiers such as IL-12, IL-21, GM-CSF, IFN-α, IFN-β and IFN-γ; treatment using therapeutic vaccines such as sipuleucel-T; treatment using dendritic cell vaccines, or tumor antigen peptide vaccines; treatment using chimeric antigen receptor (CAR)-T cells; treatment using CAR-NK cells; treatment using tumor infiltrating lymphocytes (TILs); treatment using adoptively transferred anti-tumor T cells (ex vivo expanded and/or TCR transgenic); treatment using TALL-104 cells; and treatment using immunostimulatory agents such as Toll-like receptor (TLR) agonists CpG and imiquimod; and treatment using vaccine such as BCG; wherein the combination therapy optionally provides increased effector cell killing of tumor cells, i.e., a synergy exists between the ADC constructs and the immunotherapy when co-administered.

In another aspect, there are provided novel compounds described herein as well as methods of making thereof.

### BRIEF DESCRIPTION OF THE DRAWINGS

FIG. 1 depicts the structures of Maytansine and Ansamitocins.

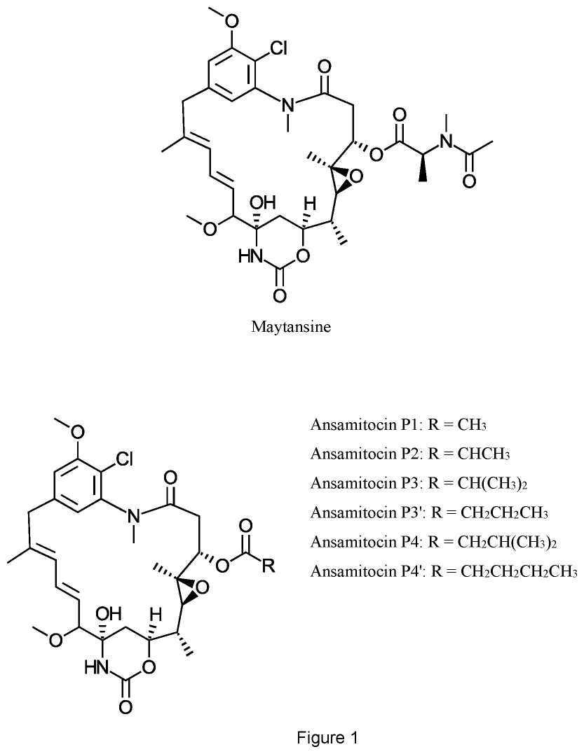

FIG. 2 depicts the structures of DM-1 and DM-4.

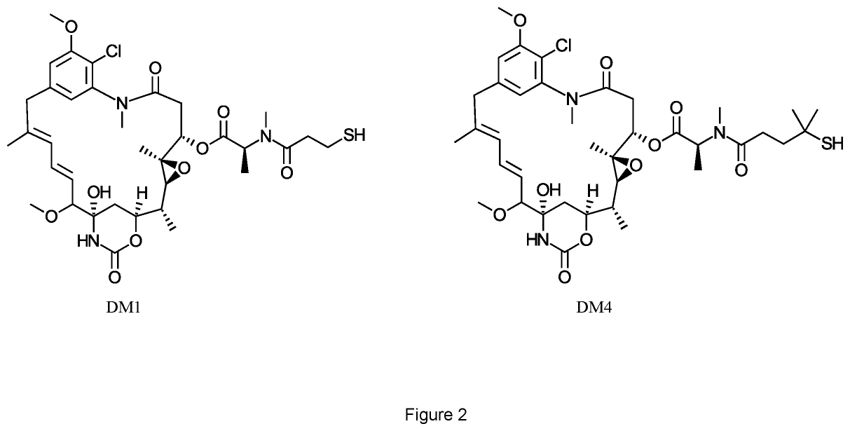

FIG. 3 depicts synthesis of compound BI-P204.

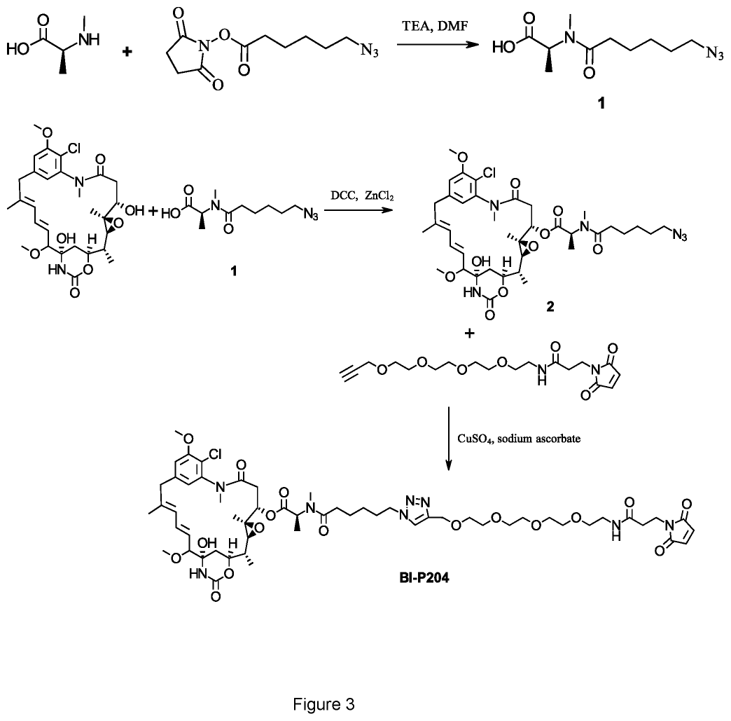

FIG. 4 depicts synthesis of compound BI-P203.

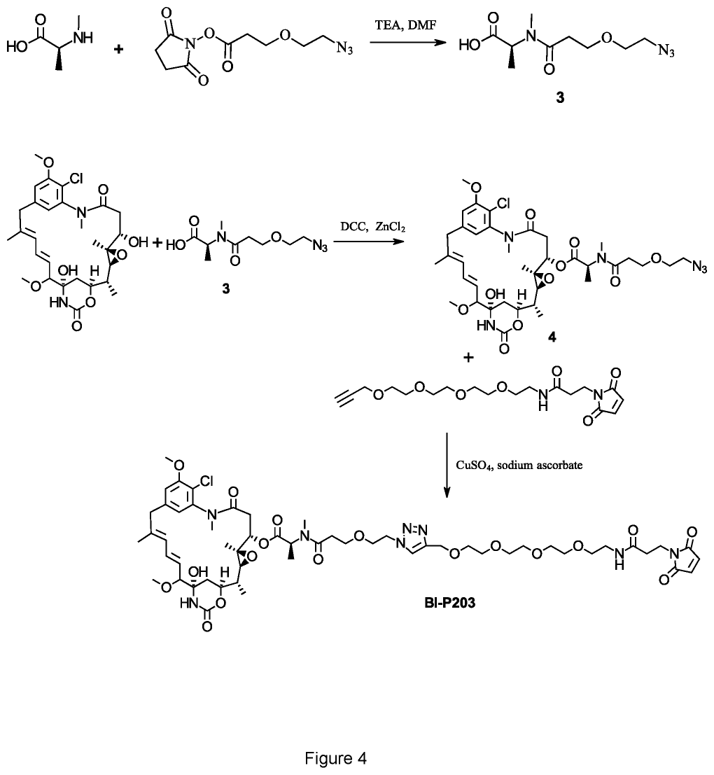

FIG. 5 depicts synthesis of compound BI-P205.

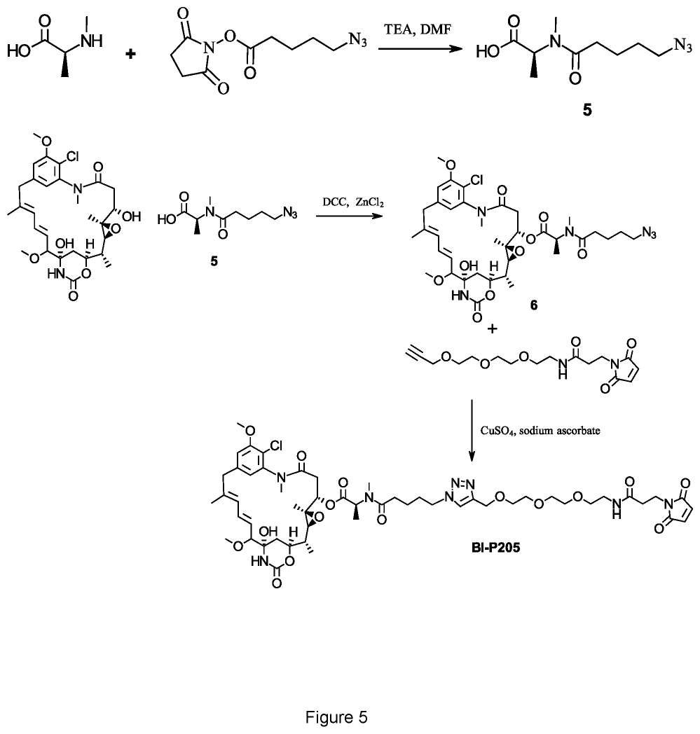

FIG. 6 depicts synthesis of compound BI-P206.

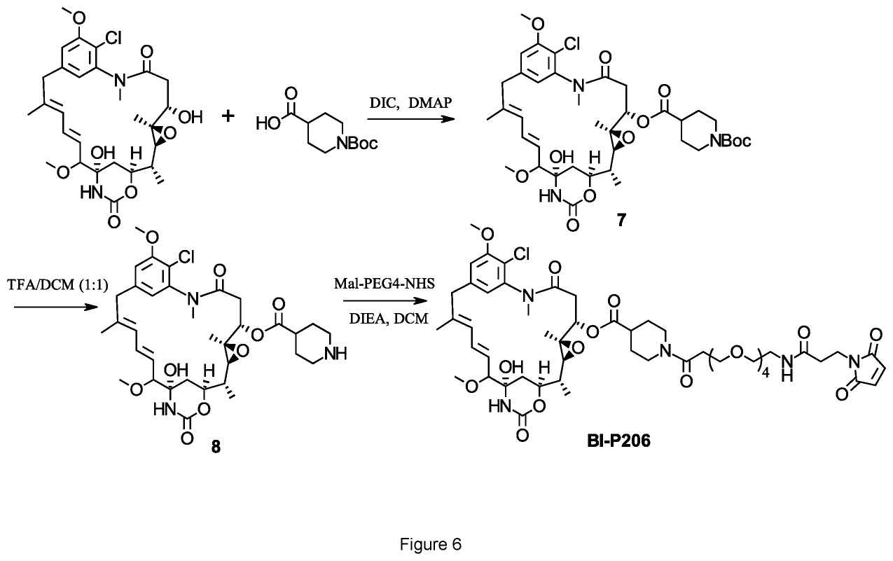

FIG. 7 depicts synthesis of compound BI-P207.

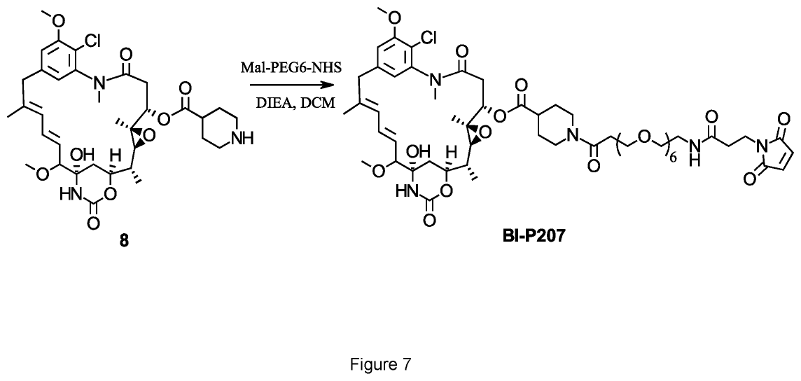

FIG. 8 depicts synthesis of compound BI-P208.

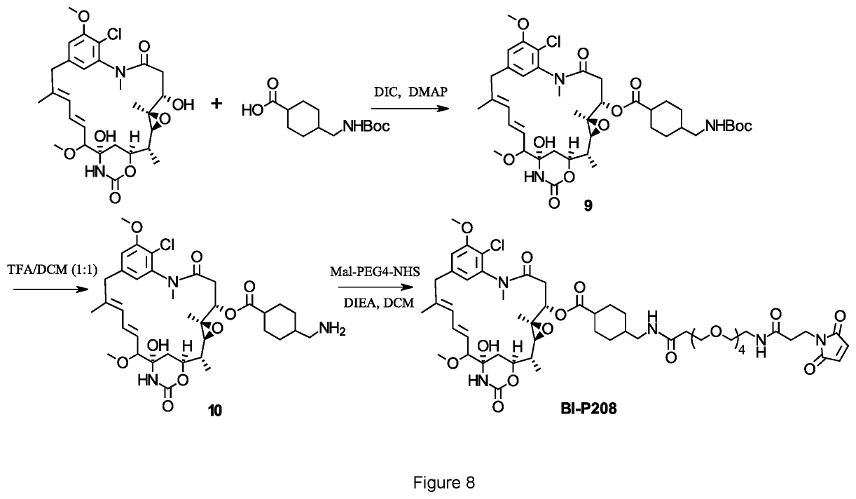

FIG. 9 depicts synthesis of compound BI-P209.

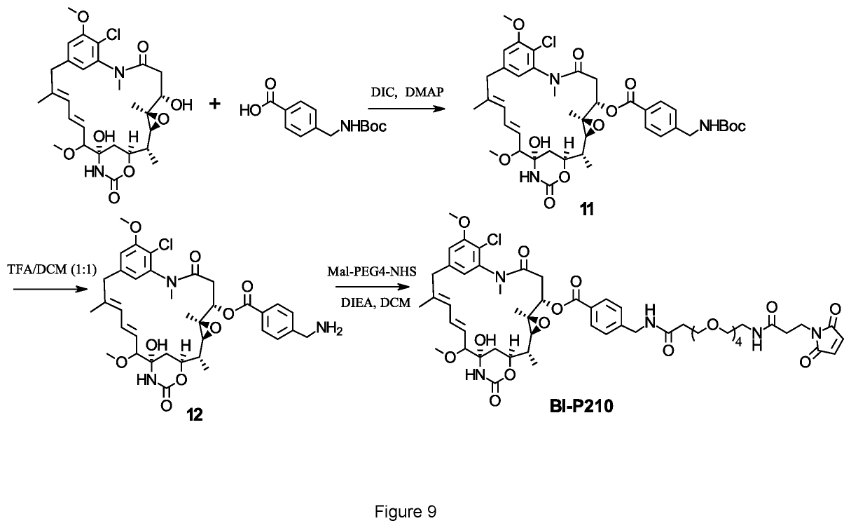

FIG. 10 depicts synthesis of compound BI-P210.

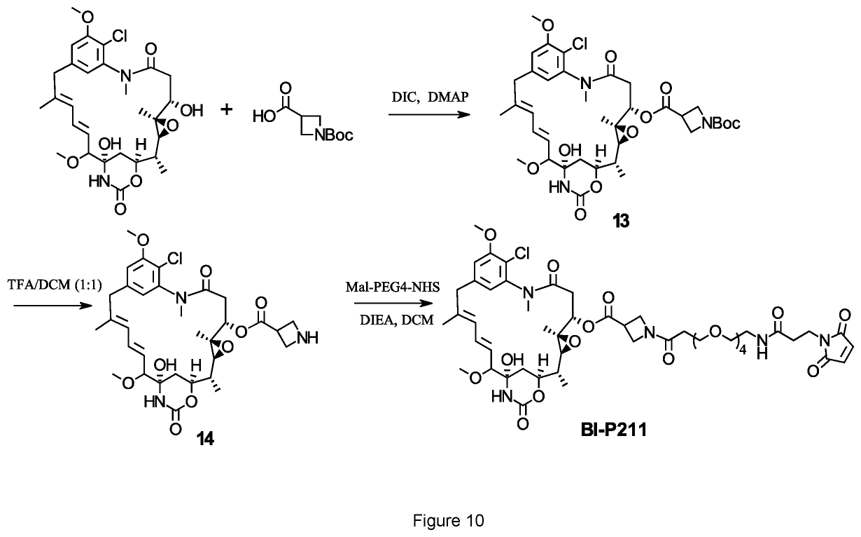

FIG. 11 depicts synthesis of compound BI-P211.

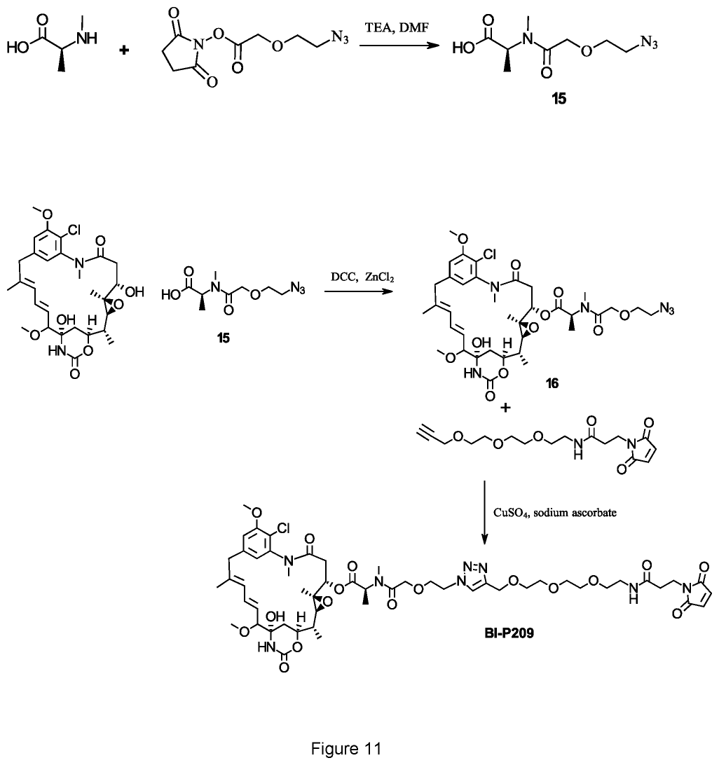

FIG. 12 depicts conjugation procedure for maytansinoid payload with reduced mAb.

FIG. 13 depicts line graphs depicting the results of in vitro cytotoxicity assay of ADCs containing various DM1 derivatives in Trop-2 positive pancreatic cancer cell line BxPC-3.

FIG. 14 depicts line graphs depicting the results of in vitro cytotoxicity assay of ADCs containing various DM1 derivatives in Trop-2 positive breast cancer cell line MDA-M B-468.

FIG. 15 depicts line graphs depicting the results of in vitro cytotoxicity assay of ADCs containing various DM1 derivatives in Trop-2 positive gastric cancer cell line NCI-N87.

FIG. 16 depicts line graphs depicting the results of in vitro cytotoxicity assay of ADCs containing various DM1 derivatives in Trop-2 positive ovarian cancer cell line SK-BR-3.

FIG. 17 depicts line graphs depicting the results of in vitro cytotoxicity assay of ADCs containing various DM1 derivatives in Trop-2 positive colon cancer cell line Colo205.

FIG. 18 depicts line graphs depicting the results of in vitro cytotoxicity assay of ADCs containing various DM1 derivatives in Trop-2 negative lung cancer cell line A549.

FIG. 19 depicts line graphs depicting the results of in vitro cytotoxicity assay of ADCs containing various DM1 derivatives in Trop-2 negative breast cancer cell line MDA-MB-231.

FIG. 20 depicts line graphs depicting the results of in vitro cytotoxicity assay of ADC-BI-P203 and ADC-BI-P209 in Trop-2 positive breast cancer cell line MDA-MB-468, ovarian cancer cell line SK-BR-3, colon cancer cell line Colo205, and Trop-2 negative lung cancer cell line A549.

FIG. 21 depicts line graphs depicting the results of in vitro cytotoxicity assay of ADC-BI-P203 and ADC-BI-P209 in Trop-2 positive breast cancer cell line MDA-MB-468 (A) and Trop-2 negative lung cancer cell line A549 (B).

FIG. 22 depicts line graphs depicting the in vivo efficacy of anti-Trop-2-BI-P203 ADC in MDA-MB-468 xenograft models. FIG. 22A depicts mean tumor volume (mm³) and FIG. 22B depicts body weight (g).

FIG. 23 depicts line graphs depicting the in vivo efficacy of anti-Trop-2-BI-P203 ADC in Colo205 xenograft models. FIG. 23A depicts mean tumor volume (mm³) and FIG. 23B depicts body weight (g).

### MODE(S) OF CARRYING OUT THE INVENTION

The present invention provides novel maytansinoid drug linker derivative payloads, and novel antibody drug conjugates comprising a maytansinoid drug linker derivative payloads of the present invention linked to an antibody for targeted delivery to disease tissues. In various embodiments, the present invention provides antibody-drug conjugates (ADCs) and ADC derivatives and methods relating to the use of such conjugates to treat cancer. The antibody, or other targeting moiety in the ADC, binds to e.g., a tumor associated antigen (TAA) on the cancer cell. In various embodiments, the antibody is conjugated to a novel DM1 derivative payload which exerts a cytotoxic, cytostatic, or immunosuppressive effect on the antigen expressing cells to treat or prevent recurrence of the antigen expressing cancers or immunological disorders. Importantly, the ADCs of the present invention have superior drug/antibody ratios (DARs), demonstrate improved solubility, enhanced CMC characteristics, and increased therapeutic efficacy particularly against high antigen expressing tumors while sparing the normal tissues expressing low or no level of antigen. Moreover, the ADCs provide for the targeting of broader patient populations and patients having a refractory cancer or who previously responded to treatment with an anti-cancer therapy, but, upon cessation of therapy, suffered relapse (hereinafter “a recurrent cancer”).

### Definitions

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, immunology, microbiology, genetics and protein and nucleic acid chemistry and hybridization described herein are those commonly used and well known in the art. The methods and techniques of the present invention are generally performed according to conventional methods well known in the art and as described in various general and more specific references that are cited and discussed throughout the present specification unless otherwise indicated. See, e.g., Green and Sambrook, Molecular Cloning: A Laboratory Manual, 4th ed., Cold Spring Harbor Laboratory Press, Cold Spring Harbor, N.Y. (2012), incorporated herein by reference. Enzymatic reactions and purification techniques are performed according to manufacturer's specifications, as commonly accomplished in the art or as described herein. The nomenclature used in connection with, and the laboratory procedures and techniques of, analytical chemistry, synthetic organic chemistry, and medicinal and pharmaceutical chemistry described herein are those commonly used and well known in the art. Standard techniques are used for chemical syntheses, chemical analyses, pharmaceutical preparation, formulation, and delivery, and treatment of subjects.

As used herein, the term “alkyl” refers to a fully saturated branched or unbranched hydrocarbon moiety having up to 20 carbon atoms. Examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl and the like.

As used herein, ther term “heterocyclyl”, “heterocycloalkyl” or “heterocyclo” refers to a saturated or unsaturated non-aromatic ring or ring system, and contains at least one heteroatom selected from O, S, and N. The heterocyclyl can be attached at a heteroatom, a carbon atom, or both.

As used herein, the term “aryl” refers to an aromatic hydrocarbon group having 6-20 carbon atoms in the ring portion. Typically, aryl is monocyclic, bicyclic or tricyclic aryl having 6-20 carbon atoms. Furthermore, the term “aryl” as used herein, refers to an aromatic moiety which can be a single aromatic ring, or multiple aromatic rings that are fused together. Non-limiting examples include phenyl, naphthyl or tetrahydronaphthyl, each of which may optionally be substituted with 1-4 substituents, such as alkyl, trifluoromethyl, cycloalkyl, halogen, hydroxy, alkoxy, acyl, alkyl-C(O)—O—, aryl-O—, heteroaryl-O—, amino, thiol, alkyl-S—, aryl-S— nitro, cyano, carboxy, alkyl-O—C(O)—, carbamoyl, alkyl-S(O)—, sulfonyl, sulfonamido, phenyl, and heterocyclyl.

As used herein, “cyclic alkyl” or “cycloalkyl” refers to a saturated or unsaturated monocyclic, bicyclic or tricyclic hydrocarbon groups of 3-12 carbon atoms. Unless otherwise provided, cycloalkyl refers to cyclic hydrocarbon moiety having between 3 and 9 ring carbon atoms or between 3 and 7 ring carbon atoms, each of which can be optionally substituted with one, or two, or three, or more substituents independently selected from the group consisting of alkyl, halo, oxo, hydroxy, alkoxy, alkyl-C(O)—, acylamino, carbamoyl, alkyl-NH—, (alkyl)₂N—, thiol, alkyl-S—, nitro, cyano, carboxy, alkyl-O—C(O)—, sulfonyl, sulfonamido, sulfamoyl, and heterocyclyl.

As used herein, the term “optionally substituted” unless otherwise specified refers to a group that is unsubstituted or is substituted with one or more, typically 1, 2, 3 or 4, suitable non-hydrogen substituents.

The point of attachment of a given moiety to the parent structure can be readily determined by one of skill in art. Thus, although the point of attachment may not be explicitly shown, it would be evident to the skilled artisan based on common general knowledge in the chemical arts.

The terms “polypeptide”, “peptide” and “protein” are used interchangeably herein to refer to a polymer of amino acid residues. In various embodiments, “peptides”, “polypeptides”, and “proteins” are chains of amino acids whose alpha carbons are linked through peptide bonds. The terminal amino acid at one end of the chain (amino terminal) therefore has a free amino group, while the terminal amino acid at the other end of the chain (carboxy terminal) has a free carboxyl group. As used herein, the term “amino terminus” (abbreviated N-terminus) refers to the free α-amino group on an amino acid at the amino terminal of a peptide or to the α-amino group (imino group when participating in a peptide bond) of an amino acid at any other location within the peptide. Similarly, the term “carboxy terminus” refers to the free carboxyl group on the carboxy terminus of a peptide or the carboxyl group of an amino acid at any other location within the peptide. Peptides also include essentially any polyamino acid including, but not limited to, peptide mimetics such as amino acids joined by an ether bond as opposed to an amide bond.

Polypeptides of the invention include polypeptides that have been modified in any way and for any reason, for example, to: (1) reduce susceptibility to proteolysis, (2) reduce susceptibility to oxidation, (3) alter binding affinity for forming protein complexes, (4) alter binding affinities, and (5) confer or modify other physicochemical or functional properties. For example, single or multiple amino acid substitutions (e.g., conservative amino acid substitutions) may be made in the naturally occurring sequence (e.g., in the portion of the polypeptide outside the domain(s) forming intermolecular contacts). A “conservative amino acid substitution” refers to the substitution in a polypeptide of an amino acid with a functionally similar amino acid. A “non-conservative amino acid substitution” refers to the substitution of a member of one of these classes for a member from another class. In making such changes, according to various embodiments, the hydropathic index of amino acids may be considered. Each amino acid has been assigned a hydropathic index on the basis of its hydrophobicity and charge characteristics. They are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is understood in the art (see, for example, Kyte et al., 1982, J. Mol. Biol. 157:105-131). It is known that certain amino acids may be substituted for other amino acids having a similar hydropathic index or score and still retain a similar biological activity. In making changes based upon the hydropathic index, in various embodiments, the substitution of amino acids whose hydropathic indices are within ±2 is included. In various embodiments, those that are within ±1 are included, and in various embodiments, those within ±0.5 are included.

It is also understood in the art that the substitution of like amino acids can be made effectively on the basis of hydrophilicity, particularly where the biologically functional protein or peptide thereby created is intended for use in immunological embodiments, as disclosed herein. In various embodiments, the greatest local average hydrophilicity of a protein, as governed by the hydrophilicity of its adjacent amino acids, correlates with its immunogenicity and antigenicity, i.e., with a biological property of the protein.

The following hydrophilicity values have been assigned to these amino acid residues: arginine (+3.0); lysine (+3.0); aspartate (+3.0.+−0.1); glutamate (+3.0.+−0.1); serine (+0.3); asparagine (+0.2); glutamine (+0.2); glycine (0); threonine (−0.4); proline (−0.5.+−0.1); alanine (−0.5); histidine (−0.5); cysteine (−1.0); methionine (−1.3); valine (−1.5); leucine (−1.8); isoleucine (−1.8); tyrosine (−2.3); phenylalanine (−2.5) and tryptophan (−3.4). In making changes based upon similar hydrophilicity values, in various embodiments, the substitution of amino acids whose hydrophilicity values are within ±2 is included, in various embodiments, those that are within ±1 are included, and in various embodiments, those within ±0.5 are included.

The term “polypeptide fragment” and “truncated polypeptide” as used herein refers to a polypeptide that has an amino-terminal and/or carboxy-terminal deletion as compared to a corresponding full-length protein. In various embodiments, fragments can be, e.g., at least 5, at least 10, at least 25, at least 50, at least 100, at least 150, at least 200, at least 250, at least 300, at least 350, at least 400, at least 450, at least 500, at least 600, at least 700, at least 800, at least 900 or at least 1000 amino acids in length. In various embodiments, fragments can also be, e.g., at most 1000, at most 900, at most 800, at most 700, at most 600, at most 500, at most 450, at most 400, at most 350, at most 300, at most 250, at most 200, at most 150, at most 100, at most 50, at most 25, at most 10, or at most 5 amino acids in length. A fragment can further comprise, at either or both of its ends, one or more additional amino acids, for example, a sequence of amino acids from a different naturally-occurring protein (e.g., an Fc or leucine zipper domain) or an artificial amino acid sequence (e.g., an artificial linker sequence).

The terms “polypeptide variant” and “polypeptide mutant” as used herein refers to a polypeptide that comprises an amino acid sequence wherein one or more amino acid residues are inserted into, deleted from and/or substituted into the amino acid sequence relative to another polypeptide sequence. In various embodiments, the number of amino acid residues to be inserted, deleted, or substituted can be, e.g., at least 1, at least 2, at least 3, at least 4, at least 5, at least 10, at least 25, at least 50, at least 75, at least 100, at least 125, at least 150, at least 175, at least 200, at least 225, at least 250, at least 275, at least 300, at least 350, at least 400, at least 450 or at least 500 amino acids in length. Variants of the present invention include fusion proteins.

A “derivative” of a polypeptide is a polypeptide that has been chemically modified, e.g., conjugation to another chemical moiety such as, for example, polyethylene glycol, albumin (e.g., human serum albumin), phosphorylation, and glycosylation.

The term “% sequence identity” is used interchangeably herein with the term “% identity” and refers to the level of amino acid sequence identity between two or more peptide sequences or the level of nucleotide sequence identity between two or more nucleotide sequences, when aligned using a sequence alignment program. For example, as used herein, 80% identity means the same thing as 80% sequence identity determined by a defined algorithm and means that a given sequence is at least 80% identical to another length of another sequence. In various embodiments, the % identity is selected from, e.g., at least 60%, at least 65%, at least 70%, at least 75%, at least 80%, at least 85%, at least 90%, at least 95%, or at least 99% or more sequence identity to a given sequence. In various embodiments, the % identity is in the range of, e.g., about 60% to about 70%, about 70% to about 80%, about 80% to about 85%, about 85% to about 90%, about 90% to about 95%, or about 95% to about 99%.

The term “% sequence homology” is used interchangeably herein with the term “% homology” and refers to the level of amino acid sequence homology between two or more peptide sequences or the level of nucleotide sequence homology between two or more nucleotide sequences, when aligned using a sequence alignment program. For example, as used herein, 80% homology means the same thing as 80% sequence homology determined by a defined algorithm, and accordingly a homologue of a given sequence has greater than 80% sequence homology over a length of the given sequence. In various embodiments, the % homology is selected from, e.g., at least 60%, at least 65%, at least 70%, at least 75%, at least 80%, at least 85%, at least 90%, at least 95%, or at least 99% or more sequence homology to a given sequence. In various embodiments, the % homology is in the range of, e.g., about 60% to about 70%, about 70% to about 80%, about 80% to about 85%, about 85% to about 90%, about 90% to about 95%, or about 95% to about 99%.

Exemplary computer programs which can be used to determine identity between two sequences include, but are not limited to, the suite of BLAST programs, e.g., BLASTN, BLASTX, and TBLASTX, BLASTP and TBLASTN, publicly available on the Internet at the NCBI website. See also Altschul et al., J. Mol. Biol. 215:403-10, 1990 (with special reference to the published default setting, i.e., parameters w=4, t=17) and Altschul et al., Nucleic Acids Res., 25:3389-3402, 1997. Sequence searches are typically carried out using the BLASTP program when evaluating a given amino acid sequence relative to amino acid sequences in the GenBank Protein Sequences and other public databases. The BLASTX program is preferred for searching nucleic acid sequences that have been translated in all reading frames against amino acid sequences in the GenBank Protein Sequences and other public databases. Both BLASTP and BLASTX are run using default parameters of an open gap penalty of 11.0, and an extended gap penalty of 1.0, and utilize the BLOSUM-62 matrix. See Id.

In addition to calculating percent sequence identity, the BLAST algorithm also performs a statistical analysis of the similarity between two sequences (see, e.g., Karlin & Altschul, Proc. Nat'l. Acad. Sci. USA, 90:5873-5787, 1993). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is, e.g., less than about 0.1, less than about 0.01, or less than about 0.001.

The terms “substantial similarity” or “substantially similar,” in the context of polypeptide sequences, indicate that a polypeptide region has a sequence with at least 70%, typically at least 80%, more typically at least 85%, or at least 90% or at least 95% sequence similarity to a reference sequence. For example, a polypeptide is substantially similar to a second polypeptide, for example, where the two peptides differ by one or more conservative substitution(s).

The term “recombinant polypeptide”, as used herein, is intended to include all polypeptides, including fusion molecules and ADCs that are prepared, expressed, created, derived from, or isolated by recombinant means, such as polypeptides expressed using a recombinant expression vector transfected into a host cell.

The term “heterologous” as used herein refers to a composition or state that is not native or naturally found, for example, that may be achieved by replacing an existing natural composition or state with one that is derived from another source. Similarly, the expression of a protein in an organism other than the organism in which that protein is naturally expressed constitutes a heterologous expression system and a heterologous protein.

The term “tumor associated antigen” (TAA) refers to, e.g., cell surface antigens that are selectively expressed by cancer cells or over-expressed in cancer cells relative to most normal cells. The terms “TAA variant” and “TAA mutant” as used herein refers to a TAA that comprises an amino acid sequence wherein one or more amino acid residues are inserted into, deleted from and/or substituted into the amino acid sequence relative to another TAA sequence. In various embodiments, the number of amino acid residues to be inserted, deleted, or substituted can be, e.g., at least 1, at least 2, at least 3, at least 4, at least 5, at least 10, at least 25, at least 50, at least 75, at least 100, at least 125, at least 150, at least 175, at least 200, at least 225, at least 250, at least 275, at least 300, at least 350, at least 400, at least 450 or at least 500 amino acids in length.

The term “anti-TAA antagonist antibody” (interchangeably termed “anti-TAA antibody”) refers to an antibody that is able to bind to TAA and inhibit TAA biological activity and/or downstream pathway(s) mediated by TAA signaling. An anti-TAA antagonist antibody encompasses antibodies that block, antagonize, suppress or reduce (including significantly) TAA biological activity, including downstream pathways mediated by TAA signaling, such as receptor binding and/or elicitation of a cellular response to TAA. For purpose of the present invention, it will be explicitly understood that the term “anti-TAA antagonist antibody” encompasses all the previously identified terms, titles, and functional states and characteristics whereby the TAA itself, an TAA biological activity (including but not limited to its ability to mediate any aspect of headache), or the consequences of the biological activity, are substantially nullified, decreased, or neutralized in any meaningful degree. In some embodiment, an anti-TAA antagonist antibody binds TAA and prevents TAA binding to a TAA receptor. In other embodiments, an anti-TAA antibody binds TAA and prevents activation of a TAA receptor. Examples of anti-TAA antagonist antibodies are provided herein.

The term “[Target] antibody” should be interpreted as similar to “anti-[Target] antibody” and means an antibody capable of binding to the [Target]. The term “Target” or [Target] shall be interpreted as a TAA or any molecule present at the surface of cells, preferably tumoral cells, more preferably mammals and human cells, and which can be used for drug delivery. Preferably, the Target is specifically express or overexpress on the surface of tumoral cells in comparison with normal cells.

The term “antibody” is used herein to refer to a protein comprising one or more polypeptides substantially or partially encoded by immunoglobulin genes or fragments of immunoglobulin genes and having specificity to a tumor antigen or specificity to a molecule overexpressed in a pathological state. The recognized immunoglobulin genes include the kappa, lambda, alpha, gamma, delta, epsilon and mu constant region genes, as well as subtypes of these genes and myriad of immunoglobulin variable region genes. Light chains (LC) are classified as either kappa or lambda. Heavy chains (HC) are classified as gamma, mu, alpha, delta, or epsilon, which in turn define the immunoglobulin classes, IgG, IgM, IgA, IgD and IgE, respectively. A typical immunoglobulin (e.g., antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition.

In a full-length antibody, each heavy chain is comprised of a heavy chain variable region (abbreviated herein as HCVR or VH) and a heavy chain constant region. The heavy chain constant region is comprised of three domains, CH1, CH2 and CH3 (and in some instances, CH4). Each light chain is comprised of a light chain variable region (abbreviated herein as LCVR or VL) and a light chain constant region. The light chain constant region is comprised of one domain, C_{L}. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The extent of the framework region and CDRs has been defined. The sequences of the framework regions of different light or heavy chains are relatively conserved within a species, such as humans. The framework region of an antibody, that is the combined framework regions of the constituent light and heavy chains, serves to position and align the CDRs in three-dimensional space. Immunoglobulin molecules can be of any type (e.g., IgG, IgE, IgM, IgD, IgA and IgY), class (e.g., IgG1, IgG2, IgG 3, IgG4, IgA1 and IgA2) or subclass.

The CDRs are primarily responsible for binding to an epitope of an antigen. The CDRs of each chain are typically referred to as CDR1, CDR2, CDR3, numbered sequentially starting from the N-terminus, and are also typically identified by the chain in which the particular CDR is located. Thus, a VH CDR3 is located in the variable domain of the heavy chain of the antibody in which it is found, whereas a VL CDR1 is the CDR1 from the variable domain of the light chain of the antibody in which it is found. Antibodies with different specificities (i.e. different combining sites for different antigens) have different CDRs. Although it is the CDRs that vary from antibody to antibody, only a limited number of amino acid positions within the CDRs are directly involved in antigen binding. These positions within the CDRs are called specificity determining residues (SDRs).

The Kabat definition is a standard for numbering the residues in an antibody and is typically used to identify CDR regions. The Kabat database is now maintained online and CDR sequences can be determined, for example, see IMGT/V-QUEST programme version: 3.2.18 Mar. 29, 2011, available on the internet and Brochet, X. et al., Nucl. Acids Res. 36, W503-508, 2008). The Chothia definition is similar to the Kabat definition, but the Chothia definition takes into account positions of certain structural loop regions. See, e.g., Chothia et al., J. Mol. Biol., 196: 901-17, 1986; Chothia et al., Nature, 342: 877-83, 1989. The AbM definition uses an integrated suite of computer programs produced by Oxford Molecular Group that model antibody structure. See, e.g., Martin et al., Proc. Natl. Acad. Sci. USA, 86:9268-9272, 1989; “AbM™, A Computer Program for Modeling Variable Regions of Antibodies,” Oxford, UK; Oxford Molecular, Ltd. The AbM definition models the tertiary structure of an antibody from primary sequence using a combination of knowledge databases and ab initio methods, such as those described by Samudrala et al., “Ab lnitio Protein Structure Prediction Using a Combined Hierarchical Approach,” in PROTEINS, Structure, Function and Genetics Suppl., 3:194-198, 1999. The contact definition is based on an analysis of the available complex crystal structures. See, e.g., MacCallum et al., J. Mol. Biol., 5:732-45, 1996.

The term “Fc region” is used to define the C-terminal region of an immunoglobulin heavy chain, which may be generated by papain digestion of an intact antibody. The Fc region may be a native sequence Fc region or a variant Fc region. The Fc region of an immunoglobulin generally comprises two constant domains, a CH2 domain and a CH3 domain, and optionally comprises a CH4 domain. The Fc portion of an antibody mediates several important effector functions e.g. cytokine induction, ADCC, phagocytosis, complement dependent cytotoxicity (CDC) and half-life/clearance rate of antibody and antigen-antibody complexes (e.g., the neonatal FcR (FcRn) binds to the Fc region of IgG at acidic pH in the endosome and protects IgG from degradation, thereby contributing to the long serum half-life of IgG). Replacements of amino acid residues in the Fc portion to alter antibody effector function are known in the art (see, e.g., Winter et al., U.S. Pat. Nos. 5,648,260 and 5,624,821).

Antibodies exist as intact immunoglobulins or as a number of well characterized fragments. Such fragments include Fab fragments, Fab′ fragments, Fab₂, F(ab)′₂ fragments, single chain Fv proteins (“scFv”) and disulfide stabilized Fv proteins (“dsFv”), that bind to the target antigen. A scFv protein is a fusion protein in which a light chain variable region of an immunoglobulin and a heavy chain variable region of an immunoglobulin are bound by a linker, while in dsFvs, the chains have been mutated to introduce a disulfide bond to stabilize the association of the chains. While various antibody fragments are defined in terms of the digestion of an intact antibody, one of skill will appreciate that such fragments may be synthesized de novo either chemically or by utilizing recombinant DNA methodology. Thus, as used herein, the term antibody encompasses e.g., monoclonal antibodies (including full-length monoclonal antibodies), polyclonal antibodies, multispecific antibodies (e.g., bispecific antibodies) formed from at least two intact antibodies, human antibodies, humanized antibodies, camelised antibodies, chimeric antibodies, single-chain Fvs (scFv), single-chain antibodies, single domain antibodies, domain antibodies, Fab fragments, F(ab′)2 fragments, antibody fragments that exhibit the desired biological activity, disulfide-linked Fvs (sdFv), intrabodies, and epitope-binding fragments or antigen binding fragments of any of the above.

Papain digestion of antibodies produces two identical antigen-binding fragments, called “Fab” fragments, each with a single antigen-binding site. A “Fab fragment” comprises one light chain and the CH1 and variable regions of one heavy chain. The heavy chain of a Fab molecule cannot form a disulfide bond with another heavy chain molecule. A “Fab′ fragment” comprises one light chain and a portion of one heavy chain that contains the VH domain and the CH1 domain and also the region between the CH1 and CH2 domains, such that an interchain disulfide bond can be formed between the two heavy chains of two Fab′ fragments to form an F(ab′)2 molecule.

Pepsin treatment of an antibody yields an F(ab′)2 fragment that has two antigen-combining sites and is still capable of cross-linking antigen. A “F(ab′)2 fragment” contains two light chains and two heavy chains containing a portion of the constant region between the CH1 and CH2 domains, such that an interchain disulfide bond is formed between the two heavy chains. A F(ab′)2 fragment thus is composed of two Fab′ fragments that are held together by a disulfide bond between the two heavy chains.

The “Fv region” comprises the variable regions from both the heavy and light chains but lacks the constant regions.

“Single-chain antibodies” are Fv molecules in which the heavy and light chain variable regions have been connected by a flexible linker to form a single polypeptide chain, which forms an antigen binding region. Single chain antibodies are discussed in detail in International Patent Application Publication No. WO 88/01649, U.S. Pat. Nos. 4,946,778 and 5,260,203, the disclosures of which are incorporated by reference.

The terms “an antigen-binding fragment” and “antigen-binding protein” as used herein means any protein that binds a specified target antigen. “Antigen-binding fragment” includes but is not limited to antibodies and binding parts thereof, such as immunologically functional fragments. An exemplary antigen-binding fragment of an antibody is the heavy chain and/or light chain CDR(s), or the heavy and/or light chain variable region.

The term “immunologically functional fragment” (or simply “fragment”) of an antibody or immunoglobulin chain (heavy or light chain) antigen binding protein, as used herein, is a species of antigen binding protein comprising a portion (regardless of how that portion is obtained or synthesized) of an antibody that lacks at least some of the amino acids present in a full-length chain but which is still capable of specifically binding to an antigen. Such fragments are biologically active in that they bind to the target antigen and can compete with other antigen binding proteins, including intact antibodies, for binding to a given epitope. In some embodiments, the fragments are neutralizing fragments. In one aspect, such a fragment will retain at least one CDR present in the full-length light or heavy chain, and in some embodiments will comprise a single heavy chain and/or light chain or portion thereof. These biologically active fragments can be produced by recombinant DNA techniques or can be produced by enzymatic or chemical cleavage of antigen binding proteins, including intact antibodies. Immunologically functional immunoglobulin fragments include, but are not limited to, Fab, a diabody, Fab′, F(ab′)2, Fv, domain antibodies and single-chain antibodies, and can be derived from any mammalian source, including but not limited to human, mouse, rat, camelid or rabbit. It is further contemplated that a functional portion of the antigen binding proteins disclosed herein, for example, one or more CDRs, could be covalently bound to a second protein or to a small molecule to create a therapeutic agent directed to a particular target in the body, possessing bifunctional therapeutic properties, or having a prolonged serum half-life.

Diabodies are bivalent antibodies comprising two polypeptide chains, wherein each polypeptide chain comprises VH and VL regions joined by a linker that is too short to allow for pairing between two regions on the same chain, thus allowing each region to pair with a complementary region on another polypeptide chain (see, e.g., Holliger et al., Proc. Natl. Acad. Sci. USA, 90:6444-48, 1993; and Poljak et al., Structure, 2:1121-23, 1994). If the two polypeptide chains of a diabody are identical, then a diabody resulting from their pairing will have two identical antigen binding sites. Polypeptide chains having different sequences can be used to make a diabody with two different antigen binding sites. Similarly, tribodies and tetrabodies are antibodies comprising three and four polypeptide chains, respectively, and forming three and four antigen binding sites, respectively, which can be the same or different.

Bispecific antibodies or fragments can be of several configurations. For example, bispecific antibodies may resemble single antibodies (or antibody fragments) but have two different antigen binding sites (variable regions). In various embodiments bispecific antibodies can be produced by chemical techniques (Kranz et al., Proc. Natl. Acad. Sci. USA, 78:5807, 1981; by “polydoma” techniques (see, e.g., U.S. Pat. No. 4,474,893); or by recombinant DNA techniques. In various embodiments bispecific antibodies of the present invention can have binding specificities for at least two different epitopes at least one of which is a tumor associate antigen. In various embodiments the antibodies and fragments can also be heteroantibodies. Heteroantibodies are two or more antibodies, or antibody binding fragments (e.g., Fab) linked together, each antibody or fragment having a different specificity.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigen. Furthermore, in contrast to polyclonal antibody preparations that typically include different antibodies directed against different determinants (epitopes), each monoclonal antibody is directed against a single determinant on the antigen. The modifier “monoclonal” is not to be construed as requiring production of the antibody by any specific method.

The term “chimeric antibody” as used herein refers to an antibody which has framework residues from one species, such as human, and CDRs (which generally confer antigen binding) from another species, such as a murine antibody that specifically binds targeted antigen.

The term “human antibody”, as used herein, is intended to include antibodies having variable and constant regions derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include antibodies in which CDR sequences derived from the germline of another mammalian species, such as a mouse, have been grafted onto human framework sequences.

The term “humanized antibody” as used herein refers to an antibody comprising a humanized light chain and a humanized heavy chain immunoglobulin. A humanized antibody binds to the same antigen as the donor antibody that provides the CDRs. The acceptor framework of a humanized immunoglobulin or antibody may have a limited number of substitutions by amino acids taken from the donor framework. Humanized or other monoclonal antibodies can have additional conservative amino acid substitutions which have substantially no effect on antigen binding or other immunoglobulin functions. In various embodiments, the framework regions are chosen from human germline exon X_{H}, J_{H}, V_{K} and J_{K} sequences. For example, acceptor sequences for humanization of FR of a V_{H} domain can be chosen from genuine V_{H} exons V_{H} 1-18 (Matsuda et al., Nature Genetics 3:88-94, 1993) or V_{H}1-2 (Shin et al., EMBO J. 10:3641-3645, 1991) and for the hinge region (J_{H}), exon J_{H}-6 (Mattila et al., Eur. J. Immunol. 25:2578-2582, 1995). In other examples, germline V_{K} exon B3 (Cox et al., Eur. J. Immunol. 24:827-836, 1994) and J_{K} exon J_{K}-1 (Hieter et al., J. Biol. Chem. 257:1516-1522, 1982) can be chosen as acceptor sequences for V_{L} domain humanization.

The term “recombinant human antibody”, as used herein, is intended to include all human antibodies that are prepared, expressed, created or isolated by recombinant means, such as antibodies expressed using a recombinant expression vector transfected into a host cell; antibodies isolated from a recombinant, combinatorial human antibody library; antibodies isolated from an animal (e.g., a mouse) that is transgenic for human immunoglobulin genes; or antibodies prepared, expressed, created or isolated by any other means that involves splicing of human immunoglobulin gene sequences to other DNA sequences. Such recombinant human antibodies have variable and constant regions derived from human germline immunoglobulin sequences. In various embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo. All such recombinant means are well known to those of ordinary skill in the art.

The term “epitope” as used herein includes any protein determinant capable of specific binding to an immunoglobulin or T-cell receptor or otherwise interacting with a molecule. Epitopic determinants generally consist of chemically active surface groupings of molecules such as amino acids or carbohydrate or sugar side chains and generally have specific three-dimensional structural characteristics, as well as specific charge characteristics. An epitope may be “linear” or “conformational.” In a linear epitope, all the points of interaction between the protein and the interacting molecule (such as an antibody) occur linearly along the primary amino acid sequence of the protein. In a conformational epitope, the points of interaction occur across amino acid residues on the protein that are separated from one another. Once a desired epitope on an antigen is determined, it is possible to generate antibodies to that epitope, e.g., using the techniques described in the present invention. Alternatively, during the discovery process, the generation and characterization of antibodies may elucidate information about desirable epitopes. From this information, it is then possible to competitively screen antibodies for binding to the same epitope. An approach to achieve this is to conduct cross-competition studies to find antibodies that competitively bind with one another, e.g., the antibodies compete for binding to the antigen. The competition for binding to the epitope can be determined by any methods or techniques known by the person skilled in the art such as, without limitation, radioactivity, Biacore, ELISA, Flow cytometry, etc. As “which competes for binding to the epitope” it is meant a competition of at least 20%, preferentially at least 50% and more preferentially at least 70%.

An antigen binding protein, including an antibody, “specifically binds” to an antigen if it binds to the antigen with a high binding affinity as determined by a dissociation constant (K_{D}, or corresponding Kb, as defined below) value of at least 1×10⁻⁶ M, or at least 1×10⁻⁷ M, or at least 1×10⁻⁸ M, or at least 1×10⁻⁹ M, or at least 1×10⁻¹⁰ M, or at least 1×10⁻¹¹ M. An antigen binding protein that specifically binds to the human antigen of interest may be able to bind to the same antigen of interest from other species as well, with the same or different affinities. The term “K_{D}” as used herein refers to the equilibrium dissociation constant of a specific antibody-antigen interaction.

The term “pharmaceutical composition” refers to a composition suitable for pharmaceutical use in an animal. A pharmaceutical composition comprises a pharmacologically effective amount of an active agent and a pharmaceutically acceptable carrier. “Pharmacologically effective amount” refers to that amount of an agent effective to produce the intended pharmacological result. “Pharmaceutically acceptable carrier” refers to any of the standard pharmaceutical carriers, vehicles, buffers, and excipients, such as a phosphate buffered saline solution, 5% aqueous solution of dextrose, and emulsions, such as an oil/water or water/oil emulsion, and various types of wetting agents and/or adjuvants. Suitable pharmaceutical carriers and formulations are described in Remington's Pharmaceutical Sciences, 21st Ed. 2005, Mack Publishing Co, Easton. A “pharmaceutically acceptable salt” is a salt that can be formulated into a compound for pharmaceutical use including, e.g., metal salts (sodium, potassium, magnesium, calcium, etc.) and salts of ammonia or organic amines.

The terms “treat”, “treating” and “treatment” refer to a method of alleviating or abrogating a biological disorder and/or at least one of its attendant symptoms. As used herein, to “alleviate” a disease, disorder or condition means reducing the severity and/or occurrence frequency of the symptoms of the disease, disorder, or condition. As used herein, “treatment” is an approach for obtaining beneficial or desired clinical results. For purposes of this invention, beneficial or desired clinical results include, but are not limited to, any one or more of: alleviation of one or more symptoms, diminishment of extent of disease, preventing or delaying spread (e.g., metastasis, for example metastasis to the lung or to the lymph node) of disease, preventing or delaying recurrence of disease, delay or slowing of disease progression, amelioration of the disease state, and remission (whether partial or total). Also encompassed by “treatment” is a reduction of pathological consequence of a proliferative disease. The methods of the invention contemplate any one or more of these aspects of treatment.

The term “effective amount” or “therapeutically effective amount” as used herein refers to an amount of a compound or composition sufficient to treat a specified disorder, condition or disease such as ameliorate, palliate, lessen, and/or delay one or more of its symptoms. In reference to cancers or other unwanted cell proliferation, an effective amount comprises an amount sufficient to: (i) reduce the number of cancer cells; (ii) reduce tumor size; (iii) inhibit, retard, slow to some extent and preferably stop cancer cell infiltration into peripheral organs; (iv) inhibit (i.e., slow to some extent and preferably stop) tumor metastasis; (v) inhibit tumor growth; (vi) prevent or delay occurrence and/or recurrence of tumor; and/or (vii) relieve to some extent one or more of the symptoms associated with the cancer. An effective amount can be administered in one or more administrations.

The term “half maximal effective concentration” (EC₅₀) corresponds to the concentration of a drug, antibody or toxicant which induces a response halfway between the baseline and maximum after some specified exposure time. It is commonly used as a measure of drug's potency. The EC₅₀ of a graded dose response curve therefore represents the concentration of a compound where 50% of its maximal effect is observed. The EC₅₀ of a quantal dose response curve represents the concentration of a compound where 50% of the population exhibits a response, after specified exposure duration. Concentration measures typically follow a sigmoidal curve, increasing rapidly over a relatively small change in concentration. This can be determined mathematically by derivation of the best-fit line.

“Adjuvant setting” refers to a clinical setting in which an individual has had a history of a proliferative disease, particularly cancer, and generally (but not necessarily) been responsive to therapy, which includes, but is not limited to, surgery (such as surgical resection), radiotherapy, and chemotherapy. However, because of their history of the proliferative disease (such as cancer), these individuals are considered at risk of development of the disease. Treatment or administration in the “adjuvant setting” refers to a subsequent mode of treatment. The degree of risk (i.e., when an individual in the adjuvant setting is considered as “high risk” or “low risk”) depends upon several factors, most usually the extent of disease when first treated.

As used herein, the terms “co-administration”, “co-administered” and “in combination with”, referring to the fusion molecules of the invention and one or more other therapeutic agents, is intended to mean, and does refer to and include the following: simultaneous administration of such combination of fusion molecules of the invention and therapeutic agent(s) to an individual in need of treatment, when such components are formulated together into a single dosage form which releases said components at substantially the same time to said individual; substantially simultaneous administration of such combination of fusion molecules of the invention and therapeutic agent(s) to an individual in need of treatment, when such components are formulated apart from each other into separate dosage forms which are taken at substantially the same time by said individual, whereupon said components are released at substantially the same time to said individual; sequential administration of such combination of fusion molecules of the invention and therapeutic agent(s) to an individual in need of treatment, when such components are formulated apart from each other into separate dosage forms which are taken at consecutive times by said individual with a significant time interval between each administration, whereupon said components are released at substantially different times to said individual; and sequential administration of such combination of fusion molecules of the invention and therapeutic agent(s) to an individual in need of treatment, when such components are formulated together into a single dosage form which releases said components in a controlled manner whereupon they are concurrently, consecutively, and/or overlappingly released at the same and/or different times to said individual, where each part may be administered by either the same or a different route.

The term “therapeutic protein” refers to proteins, polypeptides, antibodies, peptides or fragments or variants thereof, having one or more therapeutic and/or biological activities. Therapeutic proteins encompassed by the invention include but are not limited to, proteins, polypeptides, peptides, antibodies, and biologics (the terms peptides, proteins, and polypeptides are used interchangeably herein). It is specifically contemplated that the term “therapeutic protein” encompasses the fusion molecules of the present invention.

The terms “patient,” “individual,” and “subject” may be used interchangeably and refer to a mammal, preferably a human or a non-human primate, but also domesticated mammals (e.g., canine or feline), laboratory mammals (e.g., mouse, rat, rabbit, hamster, guinea pig), and agricultural mammals (e.g., equine, bovine, porcine, ovine). In various embodiments, the patient can be a human (e.g., adult male, adult female, adolescent male, adolescent female, male child, female child) under the care of a physician or other health worker in a hospital, psychiatric care facility, as an outpatient, or other clinical context. In various embodiments, the patient may be an immunocompromised patient or a patient with a weakened immune system including, but not limited to patients having primary immune deficiency, AIDS; cancer and transplant patients who are taking certain immunosuppressive drugs; and those with inherited diseases that affect the immune system (e.g., congenital agammaglobulinemia, congenital IgA deficiency). In various embodiments, the patient has an immunogenic cancer, including, but not limited to bladder cancer, lung cancer, melanoma, and other cancers reported to have a high rate of mutations (Lawrence et al., Nature, 499(7457): 214-218, 2013).

The phrase “administering” or “cause to be administered” refers to the actions taken by a medical professional (e.g., a physician), or a person controlling medical care of a patient, that control and/or permit the administration of the agent(s)/compound(s) at issue to the patient. Causing to be administered can involve diagnosis and/or determination of an appropriate therapeutic regimen, and/or prescribing particular agent(s)/compounds for a patient. Such prescribing can include, for example, drafting a prescription form, annotating a medical record, and the like. Where administration is described herein, “causing to be administered” is also contemplated.

“Resistant or refractory cancer” refers to tumor cells or cancer that do not respond to previous anti-cancer therapy including, e.g., chemotherapy, surgery, radiation therapy, stem cell transplantation, and immunotherapy. Tumor cells can be resistant or refractory at the beginning of treatment, or they may become resistant or refractory during treatment. Refractory tumor cells include tumors that do not respond at the onset of treatment or respond initially for a short period but fail to respond to treatment. Refractory tumor cells also include tumors that respond to treatment with anticancer therapy but fail to respond to subsequent rounds of therapies. For purposes of this invention, refractory tumor cells also encompass tumors that appear to be inhibited by treatment with anticancer therapy but recur up to five years, sometimes up to ten years or longer after treatment is discontinued. The anticancer therapy can employ chemotherapeutic agents alone, radiation alone, targeted therapy alone, surgery alone, or combinations thereof. For ease of description and not limitation, it will be understood that the refractory tumor cells are interchangeable with resistant tumor.

The term “tumor microenvironment” refers to the cellular environment in which the tumor exists, including surrounding blood vessels, immune cells, fibroblasts, bone marrow-derived inflammatory cells, lymphocytes, signaling molecules and the extracellular matrix (ECM). Components in the tumor microenvironment can modulate the growth of tumor cells, e.g., their ability to progress and metastasize. The tumor microenvironment can also be influenced by the tumor releasing extracellular signals, promoting tumor angiogenesis and inducing peripheral immune tolerance.

The term “proliferative disease” includes tumor disease (including benign or cancerous) and/or any metastases. A proliferative disease may include hyperproliferative conditions such as hyperplasias, fibrosis (especially pulmonary, but also other types of fibrosis, such as renal fibrosis), angiogenesis, psoriasis, atherosclerosis and smooth muscle proliferation in the blood vessels, such as stenosis or restenosis following angioplasty. In some embodiments, the proliferative disease is cancer. In some embodiments, the proliferative disease is a non-cancerous disease. In some embodiments, the proliferative disease is a benign or malignant tumor.

The term “immunogenicity” as used herein refers to the ability of an antibody or antigen binding fragment to elicit an immune response (humoral or cellular) when administered to a recipient and includes, for example, the human anti-mouse antibody (HAMA) response. A HAMA response is initiated when T-cells from a subject make an immune response to the administered antibody. The T-cells then recruit B-cells to generate specific “anti-antibody” antibodies.

The term “immune cell” as used herein means any cell of hematopoietic lineage involved in regulating an immune response against an antigen (e.g., an autoantigen). In various embodiments, an immune cell is, e.g., a T cell, a B cell, a dendritic cell, a monocyte, a natural killer cell, a macrophage, Langerhan's cells, or Kuffer cells.

“Polynucleotide” refers to a polymer composed of nucleotide units. Polynucleotides include naturally occurring nucleic acids, such as deoxyribonucleic acid (“DNA”) and ribonucleic acid (“RNA”) as well as nucleic acid analogs. Nucleic acid analogs include those which include non-naturally occurring bases, nucleotides that engage in linkages with other nucleotides other than the naturally occurring phosphodiester bond or which include bases attached through linkages other than phosphodiester bonds. Thus, nucleotide analogs include, for example and without limitation, phosphorothioates, phosphorodithioates, phosphorotriesters, phosphoramidates, boranophosphates, methylphosphonates, chiral-methyl phosphonates, 2-O-methyl ribonucleotides, peptide-nucleic acids (PNAs), and the like. Such polynucleotides can be synthesized, for example, using an automated DNA synthesizer. The term “nucleic acid” typically refers to large polynucleotides. The term “oligonucleotide” typically refers to short polynucleotides, generally no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

Conventional notation is used herein to describe polynucleotide sequences: the left-hand end of a single-stranded polynucleotide sequence is the 5′-end; the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand having the same sequence as an mRNA transcribed from that DNA and which are located 5′ to the 5′-end of the RNA transcript are referred to as “upstream sequences”; sequences on the DNA strand having the same sequence as the RNA and which are 3′ to the 3′ end of the coding RNA transcript are referred to as “downstream sequences.”

“Complementary” refers to the topological compatibility or matching together of interacting surfaces of two polynucleotides. Thus, the two molecules can be described as complementary, and furthermore, the contact surface characteristics are complementary to each other. A first polynucleotide is complementary to a second polynucleotide if the nucleotide sequence of the first polynucleotide is substantially identical to the nucleotide sequence of the polynucleotide binding partner of the second polynucleotide, or if the first polynucleotide can hybridize to the second polynucleotide under stringent hybridization conditions.

“Hybridizing specifically to” or “specific hybridization” or “selectively hybridize to”, refers to the binding, duplexing, or hybridizing of a nucleic acid molecule preferentially to a particular nucleotide sequence under stringent conditions when that sequence is present in a complex mixture (e.g., total cellular) DNA or RNA. The term “stringent conditions” refers to conditions under which a probe will hybridize preferentially to its target subsequence, and to a lesser extent to, or not at all to, other sequences. “Stringent hybridization” and “stringent hybridization wash conditions” in the context of nucleic acid hybridization experiments such as Southern and northern hybridizations are sequence-dependent and are different under different environmental parameters. An extensive guide to the hybridization of nucleic acids can be found in Tijssen, 1993, Laboratory Techniques in Biochemistry and Molecular Biology—Hybridization with Nucleic Acid Probes, part I, chapter 2, “Overview of principles of hybridization and the strategy of nucleic acid probe assays”, Elsevier, N.Y.; Sambrook et al., 2001, Molecular Cloning: A Laboratory Manual, Cold Spring Harbor Laboratory, 3.sup.rd ed., NY; and Ausubel et al., eds., Current Edition, Current Protocols in Molecular Biology, Greene Publishing Associates and Wiley Interscience, NY.

Generally, highly stringent hybridization and wash conditions are selected to be about 5° C. lower than the thermal melting point (Tm) for the specific sequence at a defined ionic strength and pH. The Tm is the temperature (under defined ionic strength and pH) at which 50% of the target sequence hybridizes to a perfectly matched probe. Very stringent conditions are selected to be equal to the Tm for a particular probe. An example of stringent hybridization conditions for hybridization of complementary nucleic acids which have more than about 100 complementary residues on a filter in a Southern or northern blot is 50% formalin with 1 mg of heparin at 42° C., with the hybridization being carried out overnight. An example of highly stringent wash conditions is 0.15 M NaCl at 72° C. for about 15 minutes. An example of stringent wash conditions is a 0.2×SSC wash at 65° C. for 15 minutes. See Sambrook et al. for a description of SSC buffer. A high stringency wash can be preceded by a low stringency wash to remove background probe signal. An exemplary medium stringency wash for a duplex of, e.g., more than about 100 nucleotides, is 1×SSC at 45° C. for 15 minutes. An exemplary low stringency wash for a duplex of, e.g., more than about 100 nucleotides, is 4-6×SSC at 40° C. for 15 minutes. In general, a signal to noise ratio of 2×(or higher) than that observed for an unrelated probe in the particular hybridization assay indicates detection of a specific hybridization.

“Primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced, i.e., in the presence of nucleotides, a complementary polynucleotide template, and an agent for polymerization such as DNA polymerase. A primer is typically single-stranded but may be double-stranded. Primers are typically deoxyribonucleic acids, but a wide variety of synthetic and naturally occurring primers are useful for many applications. A primer is complementary to the template to which it is designed to hybridize to serve as a site for the initiation of synthesis but need not reflect the exact sequence of the template. In such a case, specific hybridization of the primer to the template depends on the stringency of the hybridization conditions. Primers can be labeled with, e.g., chromogenic, radioactive, or fluorescent moieties and used as detectable moieties.

“Probe,” when used in reference to a polynucleotide, refers to a polynucleotide that is capable of specifically hybridizing to a designated sequence of another polynucleotide. A probe specifically hybridizes to a target complementary polynucleotide but need not reflect the exact complementary sequence of the template. In such a case, specific hybridization of the probe to the target depends on the stringency of the hybridization conditions. Probes can be labeled with, e.g., chromogenic, radioactive, or fluorescent moieties and used as detectable moieties. In instances where a probe provides a point of initiation for synthesis of a complementary polynucleotide, a probe can also be a primer.

“Linker” refers to a molecule that joins two other molecules, either covalently, or through ionic, van der Waals or hydrogen bonds, e.g., a nucleic acid molecule that hybridizes to one complementary sequence at the 5′ end and to another complementary sequence at the 3′ end, thus joining two non-complementary sequences. A “cleavable linker” refers to a linker that can be degraded or otherwise severed to separate the two components connected by the cleavable linker. Cleavable linkers are generally cleaved by enzymes, typically peptidases, proteases, nucleases, lipases, and the like. Cleavable linkers may also be cleaved by environmental cues, such as, for example, changes in temperature, pH, salt concentration, etc. Non-cleavable linkers are linkers that release an attached payload via lysosomal degradation of the antibody following internalization.

The terms “label” or “labeled” as used herein refers to incorporation of another molecule in the antibody. In one embodiment, the label is a detectable marker, e.g., incorporation of a radiolabeled amino acid or attachment to a polypeptide of biotinyl moieties that can be detected by marked avidin (e.g., streptavidin containing a fluorescent marker or enzymatic activity that can be detected by optical or calorimetric methods). In another embodiment, the label or marker can be therapeutic, e.g., a drug conjugate or toxin. Various methods of labeling polypeptides and glycoproteins are known in the art and may be used. Examples of labels for polypeptides include, but are not limited to, the following: radioisotopes or radionuclides (e.g., ³H, ¹⁴C, ¹⁵N, ³⁵S, ⁹⁰Y, ⁹⁹Tc, ¹¹¹In, ¹²⁵I, ¹³¹I), fluorescent labels (e.g., FITC, rhodamine, lanthanide phosphors), enzymatic labels (e.g., horseradish peroxidase, β-galactosidase, luciferase, alkaline phosphatase), chemiluminescent markers, biotinyl groups, predetermined polypeptide epitopes recognized by a secondary reporter (e.g., leucine zipper pair sequences, binding sites for secondary antibodies, metal binding domains, epitope tags), magnetic agents, such as gadolinium chelates, toxins such as pertussis toxin, taxol, cytochalasin B, gramicidin D, ethidium bromide, emetine, mitomycin, etoposide, tenoposide, vincristine, vinblastine, colchicine, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. In some embodiments, labels are attached by spacer arms of various lengths to reduce potential steric hindrance.

A “vector” is a polynucleotide that can be used to introduce another nucleic acid linked to it into a cell. One type of vector is a “plasmid,” which refers to a linear or circular double stranded DNA molecule into which additional nucleic acid segments can be ligated. Another type of vector is a viral vector (e.g., replication defective retroviruses, adenoviruses and adeno-associated viruses), wherein additional DNA segments can be introduced into the viral genome. Certain vectors are capable of autonomous replication in a host cell into which they are introduced (e.g., bacterial vectors comprising a bacterial origin of replication and episomal mammalian vectors). Other vectors (e.g., non-episomal mammalian vectors) are integrated into the genome of a host cell upon introduction into the host cell, and thereby are replicated along with the host genome. An “expression vector” is a type of vector that can direct the expression of a chosen polynucleotide.

A “regulatory sequence” is a nucleic acid that affects the expression (e.g., the level, timing, or location of expression) of a nucleic acid to which it is operably linked. The regulatory sequence can, for example, exert its effects directly on the regulated nucleic acid, or through the action of one or more other molecules (e.g., polypeptides that bind to the regulatory sequence and/or the nucleic acid). Examples of regulatory sequences include promoters, enhancers and other expression control elements (e.g., polyadenylation signals). Further examples of regulatory sequences are described in, for example, Goeddel, 1990, Gene Expression Technology: Methods in Enzymology 185, Academic Press, San Diego, Calif. and Baron et al., 1995, Nucleic Acids Res. 23:3605-06. A nucleotide sequence is “operably linked” to a regulatory sequence if the regulatory sequence affects the expression (e.g., the level, timing, or location of expression) of the nucleotide sequence.

A “host cell” is a cell that can be used to express a polynucleotide of the invention. A host cell can be a prokaryote, for example, E. coli, or it can be a eukaryote, for example, a single-celled eukaryote (e.g., a yeast or other fungus), a plant cell (e.g., a tobacco or tomato plant cell), an animal cell (e.g., a human cell, a monkey cell, a hamster cell, a rat cell, a mouse cell, or an insect cell) or a hybridoma. Typically, a host cell is a cultured cell that can be transformed or transfected with a polypeptide-encoding nucleic acid, which can then be expressed in the host cell. The phrase “recombinant host cell” can be used to denote a host cell that has been transformed or transfected with a nucleic acid to be expressed. A host cell also can be a cell that comprises the nucleic acid but does not express it at a desired level unless a regulatory sequence is introduced into the host cell such that it becomes operably linked with the nucleic acid. It is understood that the term host cell refers not only to the particular subject cell but to the progeny or potential progeny of such a cell. Because certain modifications may occur in succeeding generations due to, e.g., mutation or environmental influence, such progeny may not, in fact, be identical to the parent cell, but are still included within the scope of the term as used herein.

The term “isolated molecule” (where the molecule is, for example, a polypeptide or a polynucleotide) is a molecule that by virtue of its origin or source of derivation (1) is not associated with naturally associated components that accompany it in its native state, (2) is substantially free of other molecules from the same species (3) is expressed by a cell from a different species, or (4) does not occur in nature. Thus, a molecule that is chemically synthesized, or expressed in a cellular system different from the cell from which it naturally originates, will be “isolated” from its naturally associated components. A molecule also may be rendered substantially free of naturally associated components by isolation, using purification techniques well known in the art. Molecule purity or homogeneity may be assayed by a number of means well known in the art. For example, the purity of a polypeptide sample may be assayed using polyacrylamide gel electrophoresis and staining of the gel to visualize the polypeptide using techniques well known in the art. For certain purposes, higher resolution may be provided by using HPLC or other means well known in the art for purification.

A protein or polypeptide is “substantially pure,” “substantially homogeneous,” or “substantially purified” when at least about 60% to 75% of a sample exhibits a single species of polypeptide. The polypeptide or protein may be monomeric or multimeric. A substantially pure polypeptide or protein will typically comprise about 50%, 60%, 70%, 80% or 90% W/W of a protein sample, more usually about 95%, and preferably will be over 99% pure. Protein purity or homogeneity may be indicated by a number of means well known in the art, such as polyacrylamide gel electrophoresis of a protein sample, followed by visualizing a single polypeptide band upon staining the gel with a stain well known in the art. For certain purposes, higher resolution may be provided by using HPLC or other means well known in the art for purification.

In this application, the use of the singular includes the plural unless specifically stated otherwise. In this application, the use of “or” means “and/or” unless stated otherwise. Furthermore, the use of the term “including”, as well as other forms, such as “includes” and “included”, is not limiting. Also, terms such as “element” or “component” encompass both elements and components comprising one unit and elements and components that comprise more than one subunit unless specifically stated otherwise.

Reference to “about” a value or parameter herein includes (and describes) variations that are directed to that value or parameter per se. For example, description referring to “about X” includes description of “X”.

As used herein and in the appended claims, the singular forms “a,” “or,” and “the” include plural referents unless the context clearly dictates otherwise. It is understood that aspects and variations of the invention described herein include “consisting” and/or “consisting essentially of” aspects and variations.

### Antibody Drug Conjugates

In one aspect, the present invention is directed to an ADC of the following formula (XI):

Ab-(L-D)ₙ (XI)

or a pharmaceutically acceptable salt thereof, wherein Ab is an antibody, or an antigen binding antibody fragment thereof; L is a linker; and D is a drug moiety.

In some embodiments, the ADC comprises an antibody. In some embodiments, the antibody is an anti-Trop-2 antibody. In some embodiments, the antibody comprises a heavy-chain variable domain having three complementary regions consisting of HCDR1 (SEQ ID NO: 6), HCDR2 (SEQ ID NO: 7), and HCDR3 (SEQ ID NO: 8), and a light-chain variable domain having three complementary regions consisting of LCDR1 (SEQ ID NO: 3), LCDR2 (SEQ ID NO: 4), and LCDR3 (SEQ ID NO: 5).

In some embodiments, the ADC comprises a bivalent linker. In some embodiments, the bivalent linker is L represented by formula

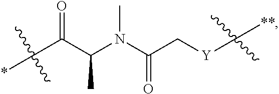

wherein * indicates the point of attachment to the drug moiety (e.g., maytasinol or maytasinol analogs), ** indicates the point of attachment to the antibody (e.g., anti-Trop-2 antibody). In some embodiments, Y is selected from the group consisting of:

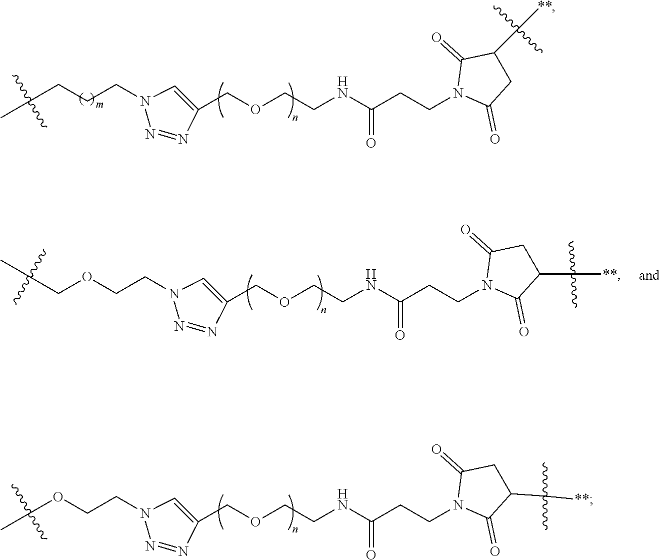

wherein m is 0-8, and n=2-12.

In some embodiments, the ADC comprises a bivalent linker L² represented by formula *—C(═O)R—Y″—**, wherein * indicates the point of attachment to the drug moiety (e.g., maytasinol or maytasinol analogs), ** indicates the point of attachment to the antibody (e.g., anti-Trop-2 antibody), R is a 3-7 membered heterocyclyl, aryl or cyclic alkyl ring. In some embodiments, L² is selected from the group consisting of:

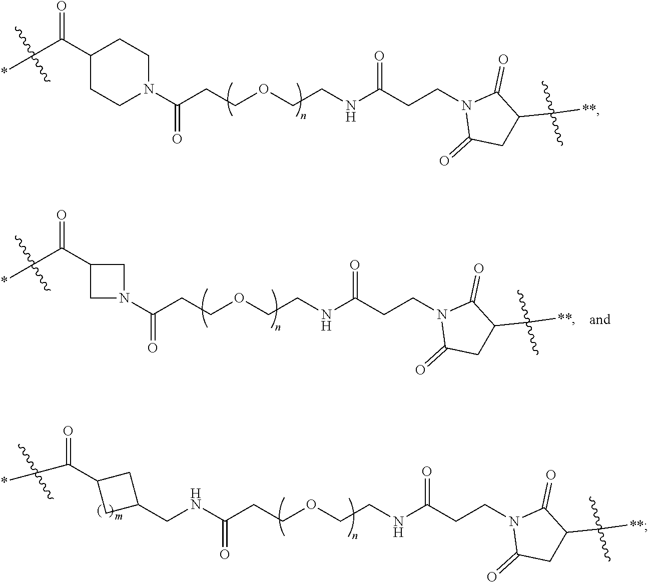

wherein m=0-3; and n=2-12.

In some embodiments, the ADC comprises an anti-Trop-2 antibody and a bivalent linker. In some embodiments, the anti-Tro-2 antibody comprises a heavy-chain variable domain having three complementary regions consisting of HCDR1 (SEQ ID NO: 6), HCDR2 (SEQ ID NO: 7), and HCDR3 (SEQ ID NO: 8), and a light-chain variable domain having three complementary regions consisting of LCDR1 (SEQ ID NO: 3), LCDR2 (SEQ ID NO: 4), and LCDR3 (SEQ ID NO: 5). In some embodiments, the bivalent linker is represented by L of formula

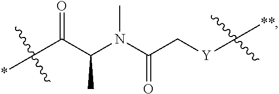

or L² of formula *—C(═O)R—Y″—**, wherein * indicates the point of attachment to the maytasinol or maytasinol analog, ** indicates the point of attachment to the anti-Trop-2 antibody, Y is selected from the group consisting of:

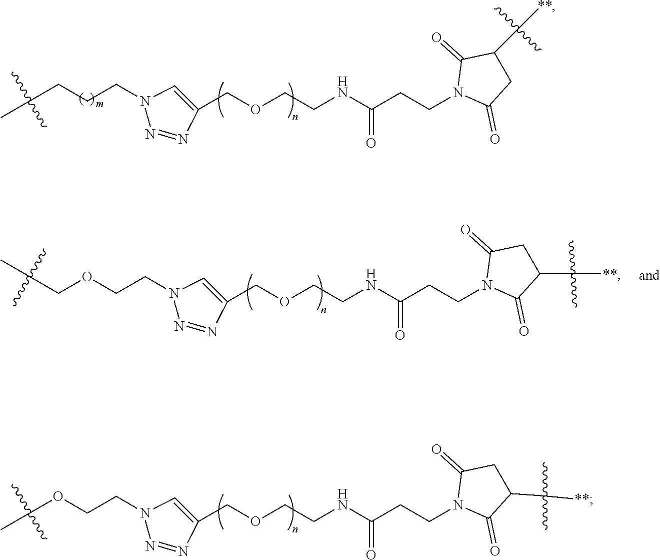

wherein m is 0-8, and n=2-12; L² is selected from the group consisting of:

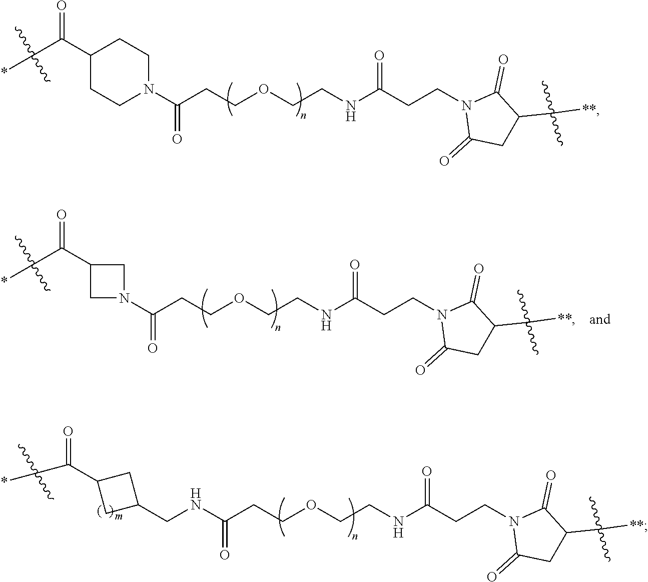

wherein m=0-3; and n=2-12.

In some embodiments, the ADC comprises an anti-Trop-2 antibody, a bivalent linker, and a maytansinol or a maytasinol analog; wherein the anti-Tro-2 antibody comprises a heavy-chain variable domain having three complementary regions consisting of HCDR1 (SEQ ID NO: 6), HCDR2 (SEQ ID NO: 7), and HCDR3 (SEQ ID NO: 8), and a light-chain variable domain having three complementary regions consisting of LCDR1 (SEQ ID NO: 3), LCDR2 (SEQ ID NO: 4), and LCDR3 (SEQ ID NO: 5); the bivalent linker is represented by L of formula

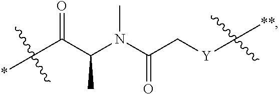

or L² of formula *—C(═O)R—Y″—**, wherein * indicates the point of attachment to the maytasinol or maytasinol analog, ** indicates the point of attachment to the anti-Trop-2 antibody, Y is selected from the group consisting of:

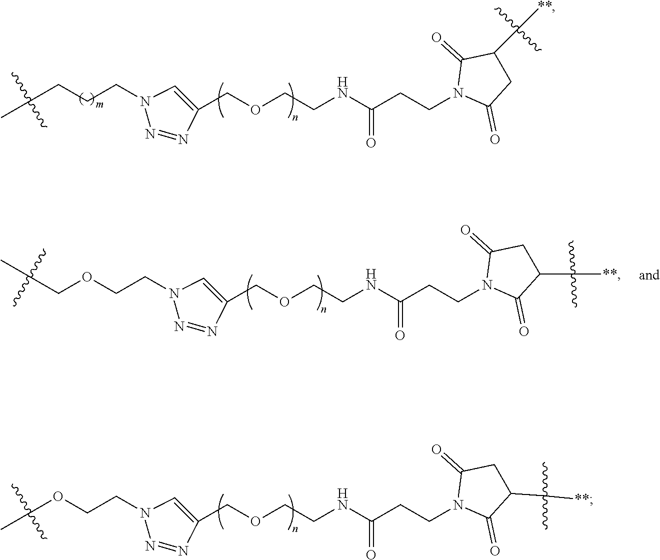

wherein m is 0-8, and n=2-12; L² is selected from the group consisting of:

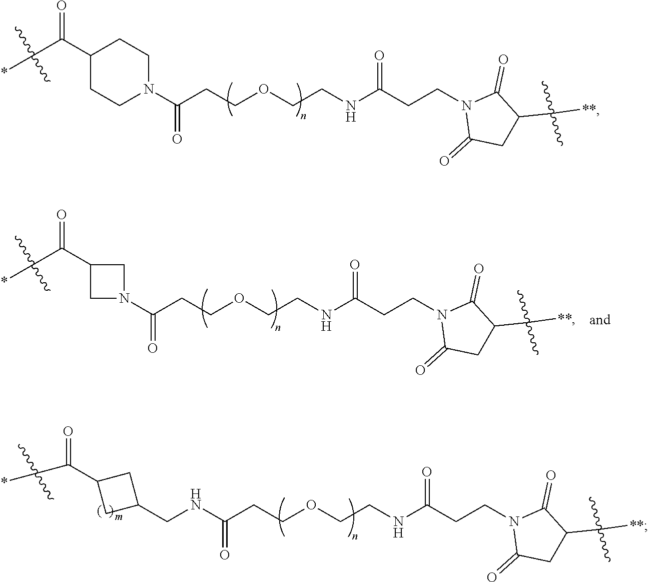

wherein m=0-3; and n=2-12.

In some embodiments, there is provided an antibody drug conjugate comprising an anti-Trop2 antibody or antigen binding fragment thereof covalently linked to a drug moiety via a bivalent linker, wherein the drug moiety and bivalent linker have the structure of:

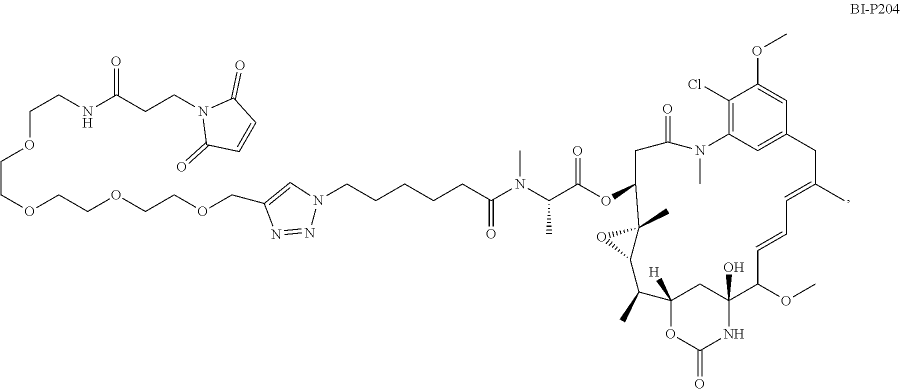 [SMILES: COc1cc2cc(c1Cl)N(C)C(=O)C[C@H](OC(=O)[C@H](C)N(C)C(=O)CCCCCn1cc(COCCOCCOCCOCCNC(=O)CCN3C(=O)C=CC3=O)nn1)[C@@]1(C)O[C@H]1[C@H](C)[C@@H]1C[C@@](O)(NC(=O)O1)C(OC)/C=C/C=C(\C)C2]

wherein the anti-Trop2 antibody comprises a heavy-chain variable domain having three complementary regions consisting of HCDR1 (SEQ ID NO: 6), HCDR2 (SEQ ID NO: 7), and HCDR3 (SEQ ID NO: 8), and a light-chain variable domain having three complementary regions consisting of LCDR1 (SEQ ID NO: 3), LCDR2 (SEQ ID NO: 4), and LCDR3 (SEQ ID NO: 5).

In some embodiments, there is provided an antibody drug conjugate comprising an anti-Trop2 antibody or antigen binding fragment thereof covalently linked to a drug moiety via a bivalent linker, wherein the drug moiety and bivalent linker have the structure of:

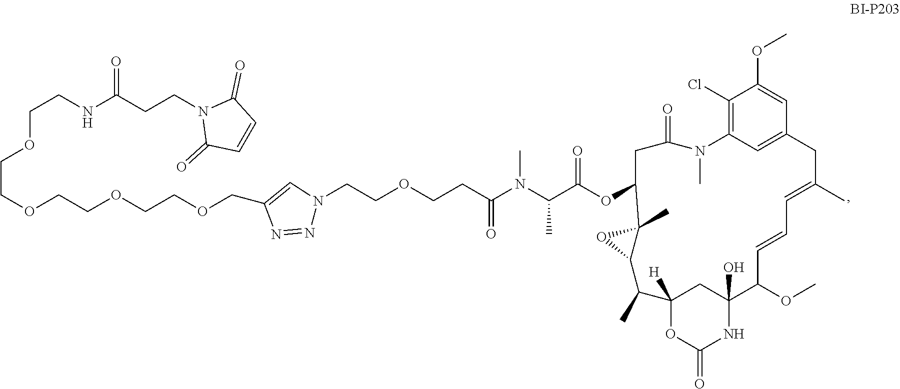 [SMILES: COc1cc2cc(c1Cl)N(C)C(=O)C[C@H](OC(=O)[C@H](C)N(C)C(=O)CCOCCn1cc(COCCOCCOCCOCCNC(=O)CCN3C(=O)C=CC3=O)nn1)[C@@]1(C)O[C@H]1[C@H](C)[C@@H]1C[C@@](O)(NC(=O)O1)C(OC)/C=C/C=C(\C)C2]

wherein the anti-Trop2 antibody comprises a heavy-chain variable domain having three complementary regions consisting of HCDR1 (SEQ ID NO: 6), HCDR2 (SEQ ID NO: 7), and HCDR3 (SEQ ID NO: 8), and a light-chain variable domain having three complementary regions consisting of LCDR1 (SEQ ID NO: 3), LCDR2 (SEQ ID NO: 4), and LCDR3 (SEQ ID NO: 5).

In some embodiments, there is provided an antibody drug conjugate comprising an anti-Trop2 antibody or antigen binding fragment thereof covalently linked to a drug moiety via a bivalent linker, wherein the drug moiety and bivalent linker have the structure of:

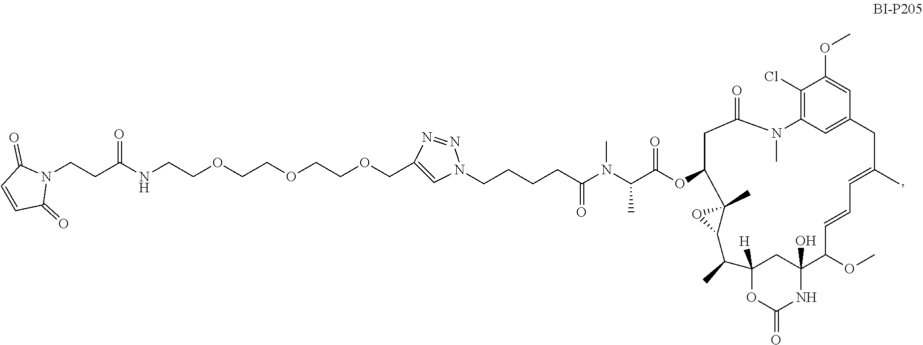 [SMILES: COc1cc2cc(c1Cl)N(C)C(=O)C[C@H](OC(=O)[C@H](C)N(C)C(=O)CCCCn1cc(COCCOCCOCCNC(=O)CCN3C(=O)C=CC3=O)nn1)[C@@]1(C)O[C@H]1[C@H](C)[C@@H]1C[C@@](O)(NC(=O)O1)C(OC)/C=C/C=C(\C)C2]

wherein the anti-Trop2 antibody comprises a heavy-chain variable domain having three complementary regions consisting of HCDR1 (SEQ ID NO: 6), HCDR2 (SEQ ID NO: 7), and HCDR3 (SEQ ID NO: 8), and a light-chain variable domain having three complementary regions consisting of LCDR1 (SEQ ID NO: 3), LCDR2 (SEQ ID NO: 4), and LCDR3 (SEQ ID NO: 5).

In some embodiments, there is provided an antibody drug conjugate comprising an anti-Trop2 antibody or antigen binding fragment thereof covalently linked to a drug moiety via a bivalent linker, wherein the drug moiety and bivalent linker have the structure of:

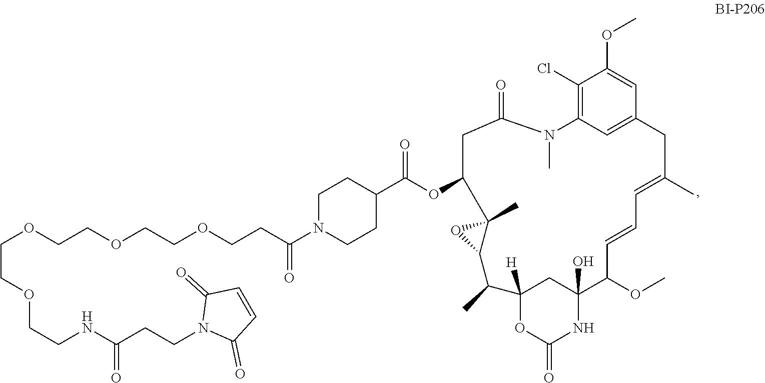 [SMILES: COc1cc2cc(c1Cl)N(C)C(=O)C[C@H](OC(=O)C1CCN(C(=O)CCOCCOCCOCCOCCNC(=O)CCN3C(=O)C=CC3=O)CC1)[C@@]1(C)O[C@H]1[C@H](C)[C@@H]1C[C@@](O)(NC(=O)O1)C(OC)/C=C/C=C(\C)C2]

wherein the anti-Trop2 antibody comprises a heavy-chain variable domain having three complementary regions consisting of HCDR1 (SEQ ID NO: 6), HCDR2 (SEQ ID NO: 7), and HCDR3 (SEQ ID NO: 8), and a light-chain variable domain having three complementary regions consisting of LCDR1 (SEQ ID NO: 3), LCDR2 (SEQ ID NO: 4), and LCDR3 (SEQ ID NO: 5).

In some embodiments, there is provided an antibody drug conjugate comprising an anti-Trop2 antibody or antigen binding fragment thereof covalently linked to a drug moiety via a bivalent linker, wherein the drug moiety and bivalent linker have the structure of:

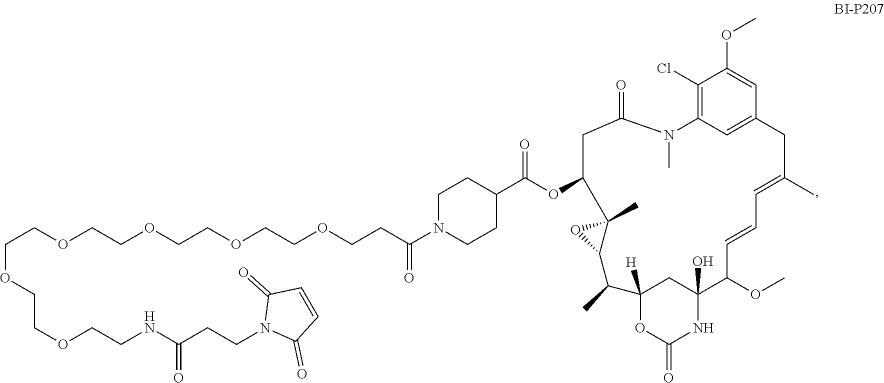 [SMILES: COc1cc2cc(c1Cl)N(C)C(=O)C[C@H](OC(=O)C1CCN(C(=O)CCOCCOCCOCCOCCOCCOCCNC(=O)CCN3C(=O)C=CC3=O)CC1)[C@@]1(C)O[C@H]1[C@H](C)[C@@H]1C[C@@](O)(NC(=O)O1)C(OC)/C=C/C=C(\C)C2]

wherein the anti-Trop2 antibody comprises a heavy-chain variable domain having three complementary regions consisting of HCDR1 (SEQ ID NO: 6), HCDR2 (SEQ ID NO: 7), and HCDR3 (SEQ ID NO: 8), and a light-chain variable domain having three complementary regions consisting of LCDR1 (SEQ ID NO: 3), LCDR2 (SEQ ID NO: 4), and LCDR3 (SEQ ID NO: 5).

In some embodiments, there is provided an antibody drug conjugate comprising an anti-Trop2 antibody or antigen binding fragment thereof covalently linked to a drug moiety via a bivalent linker, wherein the drug moiety and bivalent linker have the structure of,

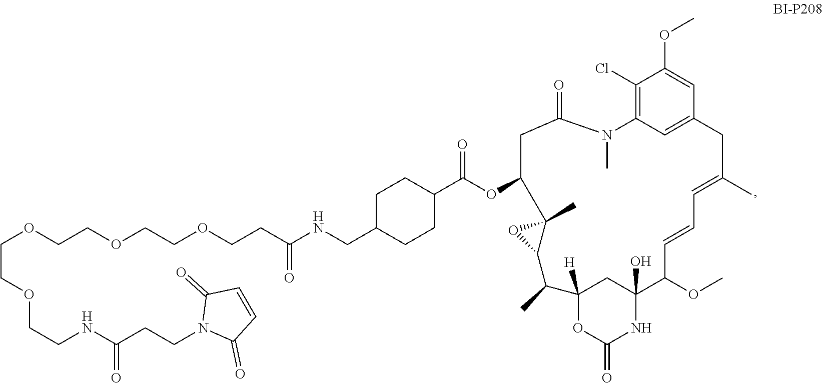 [SMILES: COc1cc2cc(c1Cl)N(C)C(=O)C[C@H](OC(=O)C1CCC(CNC(=O)CCOCCOCCOCCOCCNC(=O)CCN3C(=O)C=CC3=O)CC1)[C@@]1(C)O[C@H]1[C@H](C)[C@@H]1C[C@@](O)(NC(=O)O1)C(OC)/C=C/C=C(\C)C2]

wherein the anti-Trop2 antibody comprises a heavy-chain variable domain having three complementary regions consisting of HCDR1 (SEQ ID NO: 6), HCDR2 (SEQ ID NO: 7), and HCDR3 (SEQ ID NO: 8), and a light-chain variable domain having three complementary regions consisting of LCDR1 (SEQ ID NO: 3), LCDR2 (SEQ ID NO: 4), and LCDR3 (SEQ ID NO: 5).

In accordance with some of the methods described herein, the ADC or ADC derivative is internalized by targeted tumor cells or by activated immune cells, where the ADC or ADC derivative exerts a cytotoxic, cytostatic, or immunosuppressive effect on the antigen expressing cells to treat or prevent recurrence of the antigen expressing cancers or immunological disorders. In certain embodiments, the ADC or ADC derivative is not internalized, and the anti-Target Ab is effective to deplete or inhibiting target antigen-expressing cells by binding to the cell membrane. In certain embodiments, the ADC or ADC derivatives thereof can be targeted to a biological molecules in a cell (e.g., an inflammatory agent) and accumulate at or adjacent cells secreting or binding the biological molecule, where the therapeutic drug moiety exerts an effect (e.g., a cytotoxic, cytostatic, or immunosuppressive effect).

Importantly, the ADCs of the present invention have superior drug/antibody ratios (DARs), demonstrate improved solubility, enhanced CMC characteristics, and increased therapeutic efficacy against high antigen expressing tumor cells while having less effect on low or no antigen expressing cells i.e. normal cells. Moreover, the ADCs provide for the targeting of broader patient populations and patients with a refractory cancer or who previously responded to treatment with an anti-cancer therapy, but, upon cessation of therapy, suffered relapse (hereinafter “a recurrent cancer”).

In another aspect, the present invention is directed to a compound comprising a drug moiety (e.g. maytasinol or maytasinol analogs) and a bivalent linker. In some embodiments, the compound is a compound of formula (II) MayO-L′, or a compound of formula (VI) MayO-L²′, wherein MayO is a maytasinol or a maytasinol analog, L′ and L²′ are bivalent linkers. In some embodiments, the bivalent linker comprises a functional group which can attach to an antibody or an antigen binding fragment thereof. In some embodiments, L′ is a bivalent linker comprising a N-methylalanine moiety represented by

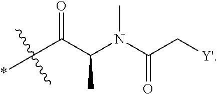

In some embodiments, Y′ is selected from a group consisting of:

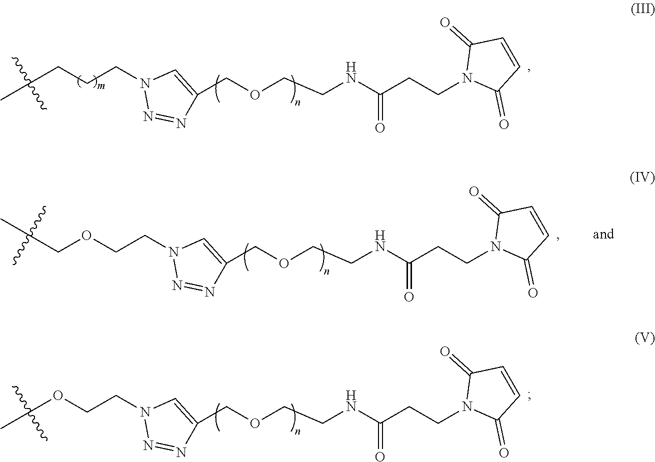

wherein m is 0 to 8; and n is 2 to 12.

In some embodiments, L²′ is a bivalent linker of formula *—C(═O)R—Y″, wherein * indicates the point of attachment to the drug moiety (e.g., maytasinol or maytasinol analogs), R is a 3-7 membered heterocyclyl, aryl or cyclic alkyl ring; and Y″ comprises a functional group which can attach to an antibody (e.g., a cell binding antibody). In some embodiment, the bivalent linker L²′ is selected from the group consisting of:

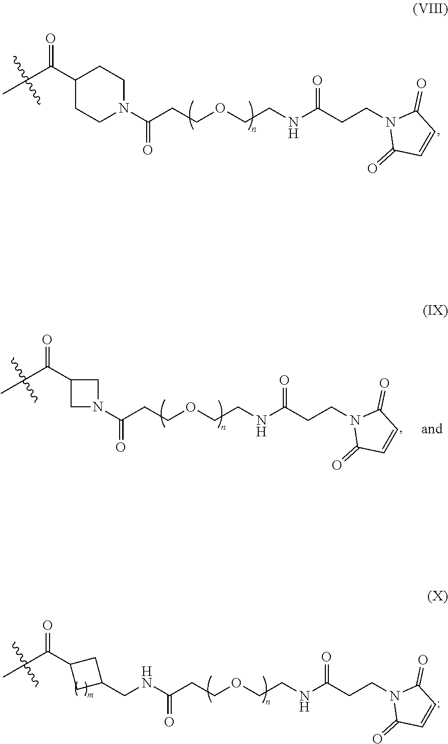

wherein m=0-3; and n=2-12.

In some embodiments, the compound comprising a drug moiety and a bivalent linker is selected from the group consisting of:

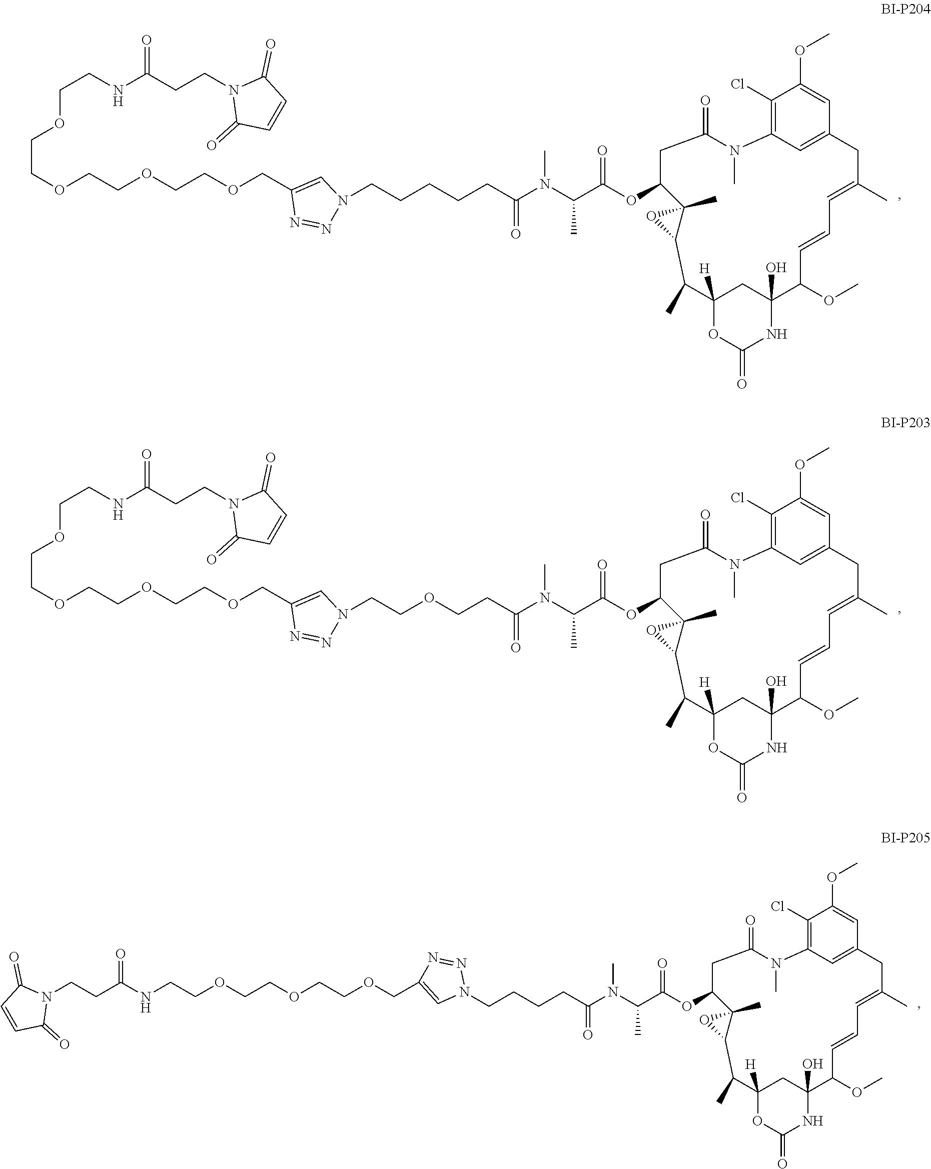

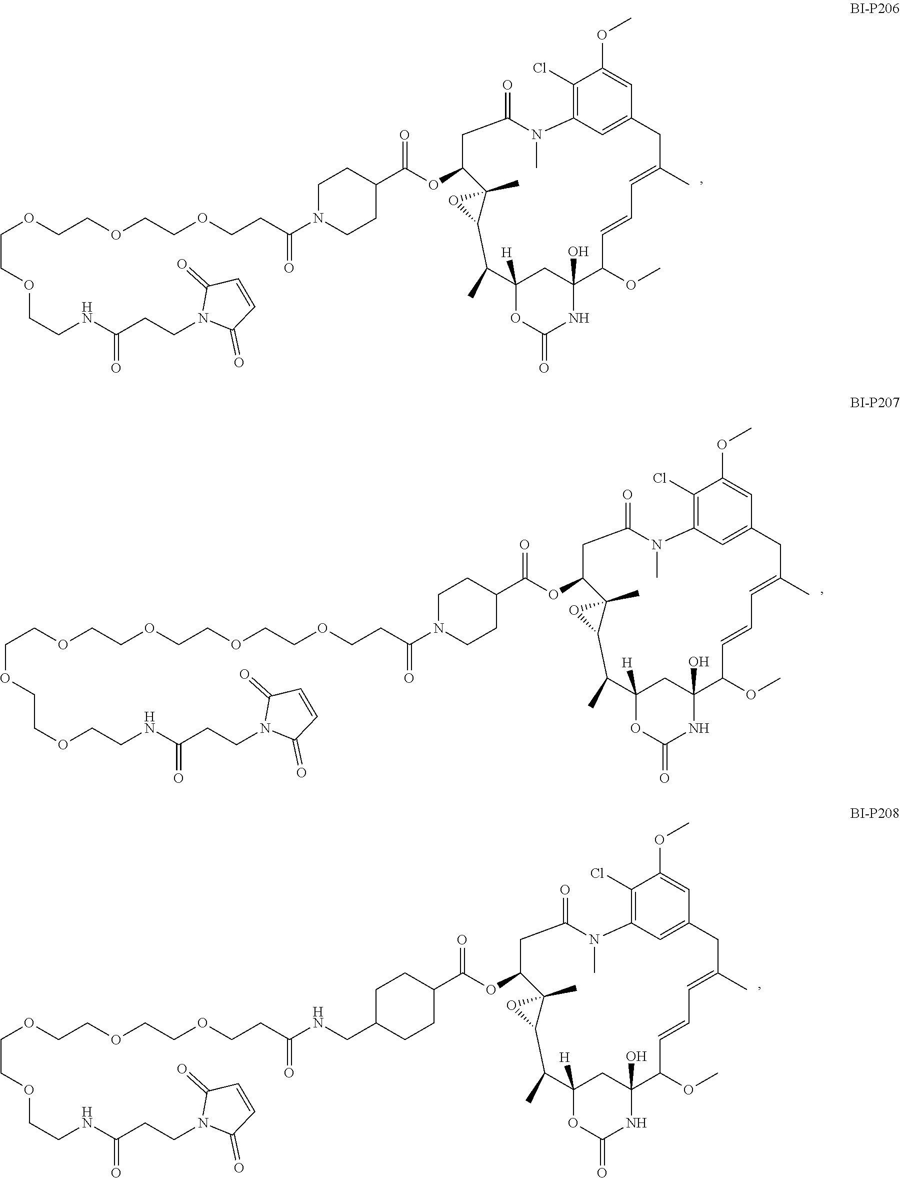

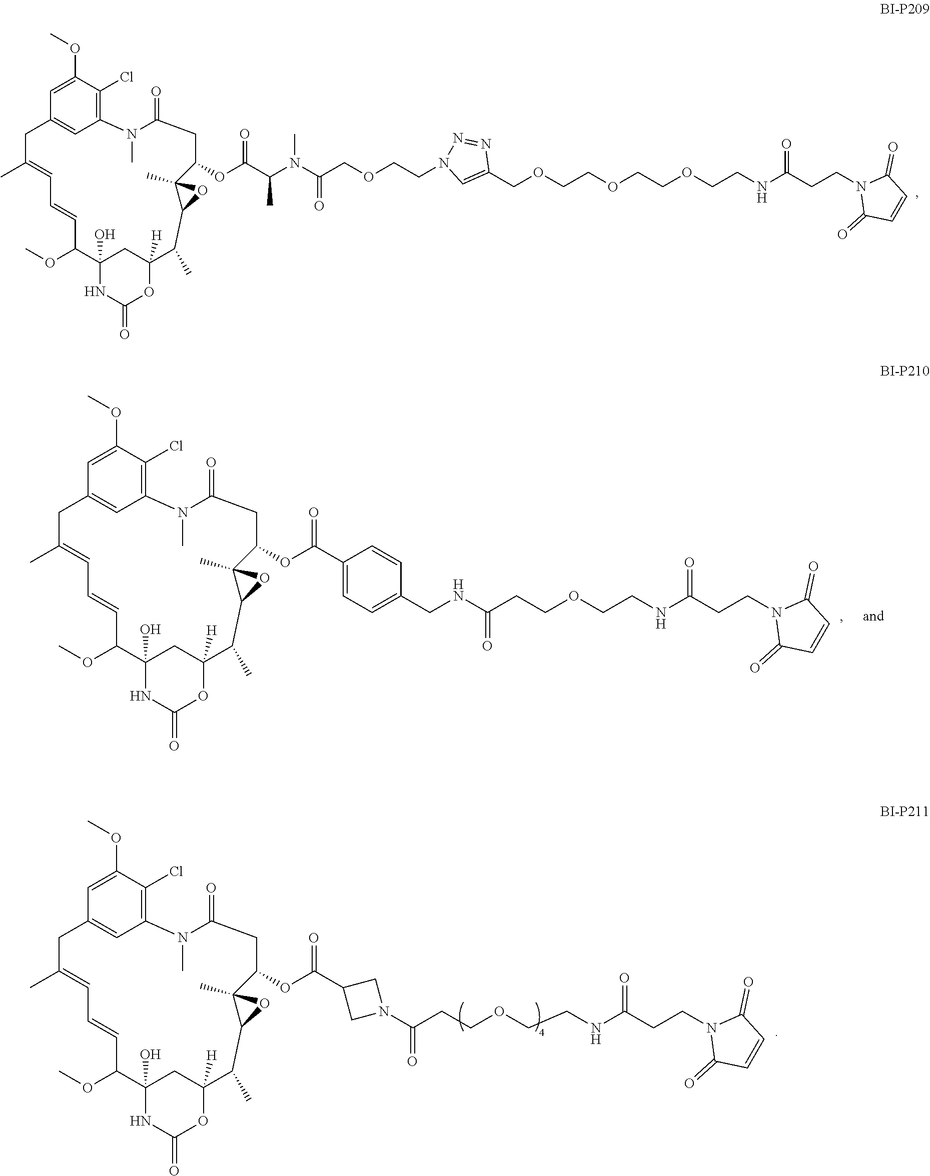

Also provided here is a method of making compounds BI-P204, BI-P203, BI-P205, BI-P206, BI-P207, BI-P208, BI-P209, BI-P210 and BI-P211 according to any of the synthesis methods described herein.

In another aspect, the present invention is directed to a method of making an antibody drug conjugate (ADC). In some embodiments, the method comprises reacting a compound with an antibody, thereby obtaining the antibody drug conjugate, wherein the compound comprises a drug moiety and a bivalent linker. In some embodiments, the compound is selected from the group consisting of compounds BI-P204, BI-P203, BI-P205, BI-P205, BI-P206, BI-P207, BI-P208, BI-P209, BI-P210 and BI-P211.

### Target Antigens and Exemplary Antibodies

Tumor antigens expressed on the cell membrane are potential targets in immunotherapy, with the ideal tumor antigen absent on normal cells and overexpressed on the tumor cell surface. The ADCs used in the methods of the present invention may comprise an antibody, or antigen binding antibody fragment, specific to any of the tumor associated antigens described in the art, including any biosimilar, biogeneric, follow-on biologic, or follow-on protein version of any TAA described in the art. The TAA can be any peptide, polypeptide, protein, nucleic acid, lipid, carbohydrate, or small organic molecule, or any combination thereof, against which the skilled artisan wishes to induce an immune response.

In various embodiments, the TAA, TAA variant, or TAA mutant contemplated for use in the combination methods of the present invention is selected from, or derived from, the list provided in Table 1.

TABLE 1

| Tumor Associated Antigen | RefSeq (protein) |
|---|---|
| Her2/neu | NP_001005862 |
| Her3 | NP_001005915 |
| Her4 | NP_001036064 |
| EGF | NP_001171601 |
| EGFR | NP_005219 |
| CD2 | NP_001758 |
| CD3 | NM_000732 |
| CD5 | NP_055022 |
| CD7 | NP_006128 |
| CD13 | NP_001141 |
| CD19 | NP_001171569 |
| CD20 | NP_068769 |
| CD21 | NP_001006659 |
| CD23 | NP_001193948 |
| CD30 | NP_001234 |
| CD33 | NP_001234.3 |
| CD34 | NP_001020280 |
| CD38 | NP_001766 |
| CD46 | NP_002380 |
| CD55 | NP_000565 |
| CD59 | NP_000602 |
| CD69 | NP_001772 |
| CD70 | NM_001252 |
| CD71 | NP_001121620 |
| CD97 | NP_001020331 |
| CD117 | NP_000213 |
| CD123 | NP_001254642 |
| CD127 | NP_002176 |
| CD134 | NP_003318 |
| CD137 | NP_001552 |
| CD138 | NP_001006947 |
| CD146 | NP_006491 |
| CD147 | NP_001719 |
| CD152 | NP_001032720 |
| CD154 | NP_000065 |
| CD174 | NP_000140 |
| CD195 | NP_000570 |
| CD200 | NP_001004196 |
| CD205 | NP_002340 |
| CD212 | NP_001276952 |
| CD223 | NP_002277 |
| CD227 | NP_001018016 |
| CD253 | NP_001177871 |
| CD272 | NP_001078826 |
| CD274 | NP_001254635 |
| CD276 | NP_001019907 |
| CD278 | NP_036224 |
| CD279 | NP_005009 |
| CD309 (VEGFR2) | NP_002244 |
| CD326 | NP_002354 |
| CD340 | NP_004439 |
| DR6 | NP_055267 |
| Kv1.3 | NP_002223 |
| 5E10 | NP_006279 |
| MUC1 | NP_001018016 |
| uPA | NM_002658 |
| SLAMF7 (CD319) | NP_001269517 |
| MAGE 3 | NP_005353 |
| MUC 16 (CA-125) | NP_078966 |
| KLK3 | NP_001025218 |
| K-ras | NP_004976 |
| Mesothelin | NP_001170826 |
| p53 | NP_000537 |
| Survivin | NP_001012270 |
| G250 (Renal Cell Carcinoma Antigen) | GenBank CAB82444.1 |
| PSMA | NP_001014986 |
| Endoplasmin (GRP94) | NM_003299 |
| BCMA | NP_001183 |
| GPNMB | NP_001005340 |
| EphA2 | NP_004422 |
| EphB2 | NP_059145 |
| TMEFF2 | NP_057276 |
| Integrin beta 6 | NP_000879 |
| 5T4 | NP_001159864 |
| CA9 | NP_001207 |
| IGF-1R | NP_000866 |
| Axl | NP_068713 |
| B7H3 | NP_001019907 |
| B7H4 | NP_078902 |
| CDH6 | NP_004923 |
| HAVCR1 | NP_001166864 |
| STEAP-1 | NP_036581 |
| STEAP-2 | NP_001035755 |
| UPK2 | NP_006751 |
| CLDN18 | NP_001002026 |

In various embodiments, the TAA has an amino acid sequence that shares an observed homology of, e.g., at least about 75%, at least about 80%, at least about 85%, at least about 90%, 91%, 92%, 93%, 94%, 95%, 96%, 97%, 98%, or at least about 99% with any one of the sequences disclosed in Table 1.

Trophoblast cell-surface antigen-2 (Trop-2), also known as tumor-associated calcium signal transducer 2 (TACSTD1), membrane component chromosome 1 surface marker 1 (M1S1), gastrointestinal antigen 733-1 (GA733-1), and epithelial glycoprotein-1 (EGP-1), belongs to the TACSTD family including at least two type I membrane proteins. It transduces an intracellular calcium signal and acts as a cell surface receptor. It has 323 amino acids, comprised of one large extracellular domain, one single transmembrane domain, and a short cytoplasmic tail. The term “Trop-2” as used herein includes human Trop-2 (hTrop-2), variants, isoforms, and species homologs of hTrop-2, and analogs having at least one common epitope with hTrop-2. In various embodiments, a hTrop-2 polypeptide as used herein may comprise the amino acid sequence set forth in NCBI Reference Sequence: NP_002344.2

Although it was first discovered as a cell surface marker of trophoblast cells, subsequent reports reveal Trop-2 is also expressed at a low level in limited normal tissues such as the nasal, breast, skin, and bronchial epithelial cells. Further studies have suggested that Trop-2 is overexpressed in many different cancer types including oral, head-and-neck, thyroid, lung, breast, gastric, colorectal, pancreatic, renal, prostate, ovarian, uterine, cervical cancers, and glioma. The elevated expression is associated with disease progression and a poor prognosis in cancer patients. Overexpression of Trop-2 in cancer cells has been shown to stimulate tumor growth both in vitro and in vivo. Similarly, inhibition of Trop-2 expression by means of siRNA has been shown to inhibit tumor cell proliferation. In addition to being critical for tumor growth, Trop-2 is also involved in metastasis. There are at least six major signaling pathways involving Trop-2, including IGF, ErbB3, ERK, MAPK, Notch-Wnt, and Raf pathways. However, its precise role in these pathways, and which downstream pathways are critical in different cancers and in different therapeutic approaches remain to be elucidated.

Based on its differential expression in tumors vs. normal tissues, its role in promoting tumor growth and metastasis, and the negative prognostic value, Trop-2 has been proposed as a promising diagnostic/therapeutic target. Blockade of Trop-2 signaling may be a means for treating cancer. A Trop-2-targeted antigen-binding fragment (Fab) has been shown to induce apoptosis and have inhibitory effects on breast cancer cell proliferation. Although several anti-Trop-2 antibodies have been developed, none was amenable to therapeutic use as naked antibodies. Trop-2-targeted antibody-drug conjugates (ADC) have been developed recently. Sacituzumab govitecan (IMMU-132) is a conjugate of humanized anti-Trop-2 antibody hRS7 with SN-38, the active metabolite of irinotecan. It has been developed and tested in many pre-clinical studies and was able to inhibit growth of a wide range of tumors. It also showed encouraging efficacy in the early phase clinical trials in non-small cell lung cancer, small cell lung cancer, metastatic triple-negative breast cancer, and pancreatic cancer patients. Although this anti-Trop-2-ADC demonstrates some clinical effect, there remains a need for improved therapeutic agents using anti-Trop-2 antibodies for the treatment of cancer and other diseases associated with Trop-2 activity. In various embodiments, the TAA is Trop-2.

Methods of generating antibodies that bind to the TAAs described herein are known to those skilled in the art. For example, a method for generating a monoclonal antibody that binds specifically to a targeted antigen polypeptide may comprise administering to a mouse an amount of an immunogenic composition comprising the targeted antigen polypeptide effective to stimulate a detectable immune response, obtaining antibody-producing cells (e.g., cells from the spleen) from the mouse and fusing the antibody-producing cells with myeloma cells to obtain antibody-producing hybridomas, and testing the antibody-producing hybridomas to identify a hybridoma that produces a monocolonal antibody that binds specifically to the targeted antigen polypeptide. Once obtained, a hybridoma can be propagated in a cell culture, optionally in culture conditions where the hybridoma-derived cells produce the monoclonal antibody that binds specifically to targeted antigen polypeptide. The monoclonal antibody may be purified from the cell culture. A variety of different techniques are then available for testing an antigen/antibody interaction to identify particularly desirable antibodies.

Other suitable methods of producing or isolating antibodies of the requisite specificity can used, including, for example, methods which select recombinant antibody from a library, or which rely upon immunization of transgenic animals (e.g., mice) capable of producing a full repertoire of human antibodies. See e.g., Jakobovits et al., Proc. Natl. Acad. Sci. (U.S.A.), 90: 2551-2555, 1993; Jakobovits et al., Nature, 362: 255-258, 1993; Lonberg et al., U.S. Pat. No. 5,545,806; and Surani et al., U.S. Pat. No. 5,545,807.

Antibodies can be engineered in numerous ways. They can be made as single-chain antibodies (including small modular immunopharmaceuticals or SMIPs™), Fab and F(ab′)₂ fragments, etc. Antibodies can be humanized, chimerized, deimmunized, or fully human. Numerous publications set forth the many types of antibodies and the methods of engineering such antibodies. For example, see U.S. Pat. Nos. 6,355,245; 6,180,370; 5,693,762; 6,407,213; 6,548,640; 5,565,332; 5,225,539; 6,103,889; and 5,260,203.

Chimeric antibodies can be produced by recombinant DNA techniques known in the art. For example, a gene encoding the Fc constant region of a murine (or other species) monoclonal antibody molecule is digested with restriction enzymes to remove the region encoding the murine Fc, and the equivalent portion of a gene encoding a human Fc constant region is substituted (see Robinson et al., International Patent Publication PCT/US86/02269; Akira, et al., European Patent Application 184,187; Taniguchi, M., European Patent Application 171,496; Morrison et al., European Patent Application 173,494; Neuberger et al., International Application WO 86/01533; Cabilly et al. U.S. Pat. No. 4,816,567; Cabilly et al., European Patent Application 125,023; Better et al., Science, 240:1041-1043, 1988; Liu et al., Proc. Natl. Acad. Sci. (U.S.A.), 84:3439-3443, 1987; Liu et al., J. Immunol., 139:3521-3526, 1987; Sun et al., Proc. Natl. Acad. Sci. (U.S.A.), 84:214-218, 1987; Nishimura et al., Canc. Res., 47:999-1005, 1987; Wood et al., Nature, 314:446-449, 1985; and Shaw et al., J. Natl Cancer Inst., 80:1553-1559, 1988).

Methods for humanizing antibodies have been described in the art. In some embodiments, a humanized antibody has one or more amino acid residues introduced from a source that is nonhuman, in addition to the nonhuman CDRs. Humanization can be essentially performed following the method of Winter and co-workers (Jones et al., Nature, 321:522-525, 1986; Riechmann et al., Nature, 332:323-327, 1988; Verhoeyen et al., Science, 239:1534-1536, 1988), by substituting hypervariable region sequences for the corresponding sequences of a human antibody. Accordingly, such “humanized” antibodies are chimeric antibodies (U.S. Pat. No. 4,816,567) wherein substantially less than an intact human variable region has been substituted by the corresponding sequence from a nonhuman species. In practice, humanized antibodies are typically human antibodies in which some hypervariable region residues and possibly some framework region residues are substituted by residues from analogous sites in rodent antibodies.

U.S. Pat. No. 5,693,761 to Queen et al, discloses a refinement on Winter et al. for humanizing antibodies, and is based on the premise that ascribes avidity loss to problems in the structural motifs in the humanized framework which, because of steric or other chemical incompatibility, interfere with the folding of the CDRs into the binding-capable conformation found in the mouse antibody. To address this problem, Queen teaches using human framework sequences closely homologous in linear peptide sequence to framework sequences of the mouse antibody to be humanized. Accordingly, the methods of Queen focus on comparing framework sequences between species. Typically, all available human variable region sequences are compared to a particular mouse sequence and the percentage identity between correspondent framework residues is calculated. The human variable region with the highest percentage is selected to provide the framework sequences for the humanizing project. Queen also teaches that it is important to retain in the humanized framework, certain amino acid residues from the mouse framework critical for supporting the CDRs in a binding-capable conformation. Potential criticality is assessed from molecular models. Candidate residues for retention are typically those adjacent in linear sequence to a CDR or physically within 6 Å of any CDR residue.

In other approaches, the importance of particular framework amino acid residues is determined experimentally once a low-avidity humanized construct is obtained, by reversion of single residues to the mouse sequence and assaying antigen binding as described by Riechmann et al, 1988. Another example approach for identifying important amino acids in framework sequences is disclosed by U.S. Pat. No. 5,821,337 to Carter et al, and by U.S. Pat. No. 5,859,205 to Adair et al. These references disclose specific Kabat residue positions in the framework, which, in a humanized antibody may require substitution with the correspondent mouse amino acid to preserve avidity.

Another method of humanizing antibodies, referred to as “framework shuffling”, relies on generating a combinatorial library with nonhuman CDR variable regions fused in frame into a pool of individual human germline frameworks (Dall'Acqua et al., Methods, 36:43, 2005). The libraries are then screened to identify clones that encode humanized antibodies which retain good binding.

The choice of human variable regions, both light and heavy, to be used in making the humanized antibodies is very important to reduce antigenicity. According to the so-called “best-fit” method, the sequence of the variable region of a rodent antibody is screened against the entire library of known human variable-domain sequences. The human sequence that is closest to that of the rodent is then accepted as the human framework region (framework region) for the humanized antibody (Sims et al., J. Immunol., 151:2296, 1993; Chothia et al., J. Mol. Biol., 196:901, 1987). Another method uses a particular framework region derived from the consensus sequence of all human antibodies of a particular subgroup of light or heavy chain variable regions. The same framework may be used for several different humanized antibodies (Carter et al., Proc. Natl. Acad. Sci. (U.S.A.), 89:4285, 1992; Presta et al., J. Immunol., 151:2623, 1993).

The choice of nonhuman residues to substitute into the human variable region can be influenced by a variety of factors. These factors include, for example, the rarity of the amino acid in a particular position, the probability of interaction with either the CDRs or the antigen, and the probability of participating in the interface between the light and heavy chain variable domain interface. (See, for example, U.S. Pat. Nos. 5,693,761, 6,632,927, and 6,639,055). One method to analyze these factors is through the use of three-dimensional models of the non-human and humanized sequences. Three-dimensional immunoglobulin models are commonly available and are familiar to those skilled in the art. Computer programs are available that illustrate and display probable three-dimensional conformational structures of selected candidate immunoglobulin sequences. Inspection of these displays permits analysis of the likely role of the residues in the functioning of the candidate immunoglobulin sequence, e.g., the analysis of residues that influence the ability of the candidate immunoglobulin to bind its antigen. In this way, nonhuman residues can be selected and substituted for human variable region residues in order to achieve the desired antibody characteristic, such as increased affinity for the target antigen(s).

Methods for making fully human antibodies have been described in the art. By way of example, a method for producing a TAA antibody or antigen-binding fragment thereof comprises the steps of synthesizing a library of human antibodies on phage, screening the library with TAA or an antibody-binding portion thereof, isolating phage that bind TAA, and obtaining the antibody from the phage. By way of another example, one method for preparing the library of antibodies for use in phage display techniques comprises the steps of immunizing a non-human animal comprising human immunoglobulin loci with TAA or an antigenic portion thereof to create an immune response, extracting antibody-producing cells from the immunized animal; isolating RNA encoding heavy and light chains of antibodies of the invention from the extracted cells, reverse transcribing the RNA to produce cDNA, amplifying the cDNA using primers, and inserting the cDNA into a phage display vector such that antibodies are expressed on the phage. Recombinant anti-TAA antibodies of the invention may be obtained in this way.

Again, by way of example, recombinant human anti-TAA antibodies of the invention can also be isolated by screening a recombinant combinatorial antibody library. Preferably the library is a scFv phage display library, generated using human V_{L} and V_{H} cDNAs prepared from mRNA isolated from B cells. Methods for preparing and screening such libraries are known in the art. Kits for generating phage display libraries are commercially available (e.g., the Pharmacia Recombinant Phage Antibody System, catalog no. 27-9400-01; and the Stratagene SurfZAP™ phage display kit, catalog no. 240612). There also are other methods and reagents that can be used in generating and screening antibody display libraries (see, e.g., U.S. Pat. No. 5,223,409; PCT Publication Nos. WO 92/18619, WO 91/17271, WO 92/20791, WO 92/15679, WO 93/01288, WO 92/01047, WO 92/09690; Fuchs et al., Bio/Technology, 9:1370-1372 (1991); Hay et al., Hum. Antibod. Hybridomas, 3:81-85, 1992; Huse et al., Science, 246:1275-1281, 1989; McCafferty et al., Nature, 348:552-554, 1990; Griffiths et al., EMBO J., 12:725-734, 1993; Hawkins et al., J. Mol. Biol., 226:889-896, 1992; Clackson et al., Nature, 352:624-628, 1991; Gram et al., Proc. Natl. Acad. Sci. (U.S.A.), 89:3576-3580, 1992; Garrad et al., Bio/Technology, 9:1373-1377, 1991; Hoogenboom et al., Nuc. Acid Res., 19:4133-4137, 1991; and Barbas et al., Proc. Natl. Acad. Sci. (U.S.A.), 88:7978-7982, 1991), all incorporated herein by reference.

Human antibodies are also produced by immunizing a non-human, transgenic animal comprising within its genome some or all of human immunoglobulin heavy chain and light chain loci with a human IgE antigen, e.g., a XenoMouse™ animal (Abgenix, Inc./Amgen, Inc.- Fremont, Calif.). XenoMouse™ mice are engineered mouse strains that comprise large fragments of human immunoglobulin heavy chain and light chain loci and are deficient in mouse antibody production. See, e.g., Green et al., Nature Genetics, 7:13-21, 1994 and U.S. Pat. Nos. 5,916,771, 5,939,598, 5,985,615, 5,998,209, 6,075,181, 6,091,001, 6,114,598, 6,130,364, 6,162,963 and 6,150,584. XenoMouse™ mice produce an adult-like human repertoire of fully human antibodies and generate antigen-specific human antibodies. In some embodiments, the XenoMouse™ mice contain approximately 80% of the human antibody V gene repertoire through introduction of megabase sized, germline configuration fragments of the human heavy chain loci and kappa light chain loci in yeast artificial chromosome (YAC). In other embodiments, XenoMouse™ mice further contain approximately all of the human lambda light chain locus. See Mendez et al., Nature Genetics, 15:146-156, 1997; Green and Jakobovits, J. Exp. Med., 188:483-495, 1998; and WO 98/24893.

In various embodiments, the ADCs of the present invention utilize an antibody or antigen-binding fragment thereof is a polyclonal antibody, a monoclonal antibody or antigen-binding fragment thereof, a recombinant antibody, a diabody, a chimerized or chimeric antibody or antigen-binding fragment thereof, a humanized antibody or antigen-binding fragment thereof, a fully human antibody or antigen-binding fragment thereof, a CDR-grafted antibody or antigen-binding fragment thereof, a single chain antibody, an Fv, an Fd, an Fab, an Fab′, or an F(ab′)2, and synthetic or semi-synthetic antibodies.

In various embodiments, the ADCs of the present invention utilize an antibody or antigen-binding fragment that binds to a TAA with a dissociation constant (K_{D}) of, e.g., at least about 1×10⁻³ M, at least about 1×10⁻⁴ M, at least about 1×10⁻⁵ M, at least about 1×10⁻⁶ M, at least about 1×10⁻⁷ M, at least about 1×10⁻⁸ M, at least about 1×10⁻⁹ M, at least about 1×10⁻¹⁰ M, at least about 1×10⁻¹¹ M, or at least about 1×10⁻¹² M. In various embodiments, the fusion molecules of the present invention utilize an antibody or antigen-binding fragment that binds to a TAA with a dissociation constant (K_{D}) in the range of, e.g., at least about 1×10⁻³ M to at least about 1×10⁻⁴ M, at least about 1×10⁻⁴ M to at least about 1×10⁻⁵ M, at least about 1×10⁻⁸ M to at least about 1×10⁻⁶ M, at least about 1×10⁻⁶ M to at least about 1×10⁻⁷ M, at least about 1×10⁻⁷ M to at least about 1×10⁻⁸ M, at least about 1×10⁻⁸ M to at least about 1×10⁻⁹ M, at least about 1×10⁻⁹ M to at least about 1×10⁻¹⁰ M, at least about 1×10⁻¹° M to at least about 1×10⁻¹¹ M, or at least about 1×10⁻¹¹ M to at least about 1×10⁻¹² M.

In various embodiments, the ADCs of the present invention utilize an antibody or antigen-binding fragment that cross-competes for binding to the same epitope on the TAA as a reference antibody which comprises the heavy chain variable region and light chain variable region set forth in the references and sequence listings provided herein.

In various embodiments, antibodies contemplated for use in the ADCs of the present invention include but are not limited to LL1 (anti-CD74), LL2 or RFB4 (anti-CD22), veltuzumab (hA20, anti-CD20), rituxumab (anti-CD20), obinutuzumab (GA101, anti-CD20), lambrolizumab (anti-PD-1 receptor), nivolumab (anti-PD-1 receptor), ipilimumab (anti-CTLA-4), RS7 (anti-epithelial glycoprotein-1 (EGP-1, also known as TROP-2)), PAM4 or KC4 (both anti-mucin), MN-14 (anti-carcinoembryonic antigen (CEA, also known as CD66e or CEACAM5), MN-15 or MN-3 (anti-CEACAM6), Mu-9 (anti-colon-specific antigen-p), Immu 31 (an anti-alpha-fetoprotein), R1 (anti-IGF-1R), A19 (anti-CD19), TAG-72 (e.g., CC49), Tn, J591 or HuJ591 (anti-PSMA (prostate-specific membrane antigen)), AB-PG1-XG1-026 (anti-PSMA dimer), D2/B (anti-PSMA), G250 (an anti-carbonic anhydrase IX MAb), L243 (anti-HLA-DR) alemtuzumab (anti-CD52), bevacizumab (anti-VEGF), cetuximab (anti-EGFR), gemtuzumab (anti-CD33), ibritumomab tiuxetan (anti-CD20); panitumumab (anti-EGFR); tositumomab (anti-CD20); PAM4 (aka clivatuzumab, anti-mucin); trastuzumab (anti-ErbB2); and anti-CTLA4 antibodies include ipilimumab (Bristol-Myers Squibb) and tremelimumab (PFIZER). Such antibodies are known in the art (e.g., U.S. Pat. Nos. 5,686,072; 5,874,540; 6,107,090; 6,183,744; 6,306,393; 6,653,104; 6,730,300; 6,899,864; 6,926,893; 6,962,702; 7,074,403; 7,230,084; 7,238,785; 7,238,786; 7,256,004; 7,282,567; 7,300,655; 7,312,318; 7,585,491; 7,612,180; 7,642,239; and U.S. Patent Application Publ. Nos. 20050271671; 20060193865; 20060210475; 20070087001; the Examples section of each incorporated herein by reference.) Specific known antibodies of use include hPAM4 (U.S. Pat. No. 7,282,567), hA20 (U.S. Pat. No. 7,251,164), hA19 (U.S. Pat. No. 7,109,304), hIMMU-31 (U.S. Pat. No. 7,300,655), hLL1 (U.S. Pat. No. 7,312,318), hLL2 (U.S. Pat. No. 7,074,403), hMu-9 (U.S. Pat. No. 7,387,773), hL243 (U.S. Pat. No. 7,612,180), hMN-14 (U.S. Pat. No. 6,676,924), hMN-15 (U.S. Pat. No. 7,541,440), hR1 (U.S. patent application Ser. No. 12/772,645), hRS7 (U.S. Pat. No. 7,238,785), hMN-3 (U.S. Pat. No. 7,541,440), AB-PG1-XG1-026 (U.S. patent application Ser. No. 11/983,372, deposited as ATCC PTA-4405 and PTA-4406) and D2/B (WO 2009/130575) the text of each recited patent or application is incorporated herein by reference.

In various embodiments, the ADCs of the present invention include at least one antibody or fragment thereof that binds to Trop-2. In various embodiments, the anti-Trop-2 antibody is a humanized antibody comprising the light chain sequence of SEQ ID NO: 1 and a heavy chain sequence of SEQ ID NO: 2. In various embodiments, the anti-Trop-2 antibody is a humanized antibody comprising the light chain CDR sequences CDR1 (SEQ ID NO: 3); CDR2 (SEQ ID NO: 4); and CDR3 (SEQ ID NO: 5) and the heavy chain CDR sequences CDR1 (SEQ ID NO: 6); CDR2 (SEQ ID NO: 7) and CDR3 (SEQ ID NO: 8).

In another aspect, the present invention features bispecific molecules comprising an anti-Trop-2 antibody, or antigen-binding fragment thereof, of the invention. An antibody of the invention, or antigen-binding fragment thereof, can be derivatized or linked to another functional molecule, e.g., another peptide or protein (e.g., another antibody or ligand for a receptor) to generate a bispecific molecule that binds to at least two different binding sites or target molecules. The antibody of the invention may in fact be derivatized or linked to more than one other functional molecule to generate multispecific molecules that bind to more than two different binding sites and/or target molecules; such multispecific molecules are also intended to be encompassed by the term “bispecific molecule” as used herein. To create a bispecific molecule of the invention, an antibody of the invention can be functionally linked (e.g., by chemical coupling, genetic fusion, noncovalent association or otherwise) to one or more other binding molecules, such as another antibody, antibody fragment, peptide or binding mimetic, such that a bispecific molecule results. In various embodiments, the invention includes bispecific molecules capable of binding both to FcγR or FcαR expressing effector cells (e.g., monocytes, macrophages or polymorphonuclear cells (PMNs)), and to target cells expressing Trop-2. In such embodiments, the bispecific molecules target Trop-2 expressing cells to effector cell and trigger Fc receptor-mediated effector cell activities, e.g., phagocytosis of a Trop-2 expressing cells, antibody dependent cell-mediated cytotoxicity (ADCC), cytokine release, or generation of superoxide anion. Methods of preparing the bispecific molecules of the present invention are well known in the art.

In various embodiments, another functional molecule, e.g., another antibody or ligand for a receptor) which is linked to the anti-Trop-2 antibody, can be selected from the group consisting of: agonistic, antagonistic, or blocking antibodies to signaling molecules such as Her-2, Her-3, EGFR, IGF-R, c-Met, EphA2, EphB2, and MUC16; agonistic, antagonistic, or blocking antibodies to co-stimulatory or co-inhibitory molecoles (immune checkpoints) such as PD-1, PD-L1, OX-40, CS137, GITR, LAG3, TIM-3, and VISTA; CD3 found on T cells.

Bispecific antibodies or fragments can be of several configurations. For example, bispecific antibodies may resemble single antibodies (or antibody fragments) but have two different antigen binding sites (variable regions). In various embodiments antibodies can be produced by chemical techniques (Kranz et al., Proc. Natl. Acad. Sci. USA, 78:5807, 1981; by “polydoma” techniques (see, e.g., U.S. Pat. No. 4,474,893); or by recombinant DNA techniques. In various embodiments bispecific antibodies of the present disclosure can have binding specificities for at least two different epitopes at least one of which is a tumor associate antigen. In various embodiments the antibodies and fragments can also be heteroantibodies. Heteroantibodies are two or more antibodies, or antibody binding fragments (e.g., Fab) linked together, each antibody or fragment having a different specificity.

### Characterization of Antibody Binding to Antigen

Antibodies of the present invention can be tested for binding to target antigen by, for example, standard ELISA. As an example, microtiter plates are coated with purified target antigen in PBS, and then blocked with 5% bovine serum albumin in PBS. Dilutions of antibody (e.g., dilutions of plasma from target antigen-immunized mice) are added to each well and incubated for 1-2 hours at 37° C. The plates are washed with PBS/Tween and then incubated with secondary reagent (e.g., for human antibodies, a goat-anti-human IgG Fc-specific polyclonal reagent) conjugated to alkaline phosphatase for 1 hour at 37° C. After washing, the plates are developed with pNPP substrate (1 mg/ml) and analyzed at OD of 405-650. Preferably, mice which develop the highest titers will be used for fusions. An ELISA assay can also be used to screen for hybridomas that show positive reactivity with target antigen immunogen. Hybridomas that bind with high avidity to target antigen are subcloned and further characterized. One clone from each hybridoma, which retains the reactivity of the parent cells (by ELISA), can be chosen for making a 5-10 vial cell bank stored at −140° C., and for antibody purification.

To determine if the selected anti-target antigen monoclonal antibodies bind to unique epitopes, each antibody can be biotinylated using commercially available reagents (Pierce, Rockford, Ill.). Competition studies using unlabeled monoclonal antibodies and biotinylated monoclonal antibodies can be performed using target antigen coated-ELISA plates as described above. Biotinylated mAb binding can be detected with a strep-avidin-alkaline phosphatase probe. To determine the isotype of purified antibodies, isotype ELISAs can be performed using reagents specific for antibodies of a particular isotype. For example, to determine the isotype of a human monoclonal antibody, wells of microtiter plates can be coated with 1 μg/ml of anti-human immunoglobulin overnight at 4° C. After blocking with 1% BSA, the plates are reacted with 1 μg/ml or less of test monoclonal antibodies or purified isotype controls, at ambient temperature for one to two hours. The wells can then be reacted with either human IgG1 or human IgM-specific alkaline phosphatase-conjugated probes. Plates are developed and analyzed as described above.

Anti-target antigen human IgGs can be further tested for reactivity with target antigen by Western blotting. Briefly, target antigen can be prepared and subjected to sodium dodecyl sulfate polyacrylamide gel electrophoresis. After electrophoresis, the separated antigens are transferred to nitrocellulose membranes, blocked with 10% fetal calf serum, and probed with the monoclonal antibodies to be tested. Human IgG binding can be detected using anti-human IgG alkaline phosphatase and developed with BCIP/NBT substrate tablets (Sigma Chem. Co., St. Louis, Mo.).

The binding affinity of human IgGs to their antigens can be determined by Octet system based on Bio-Layer Interferometry (BLI) technique. BLI is a layer of molecules attached to the tip of an optic fiber which creates an interference pattern at the detector, any change in the number of molecules bound causes a measured shift in the pattern. Octet system enables real-time analysis for determination of affinity and kinetics from biomolecular interactions in 96-well microplates. Briefly, target antigen can be diluted and loaded to a 96-well microplate. Antibody is then diluted and added to the assigned wells. The plate is put in the Octet system and assay is started. The data can be analyzed using the Octet Data Acquisition Software (fortebio data analysis 10.0) to calculate an association rate constant Kon, a dissociation rate constant Koff, and a dissociation constant Kd (Kd=Koff/Kon).

The binding of human IgGs to their antigens expressed on cell surface can be tested by flow cytometry. Briefly, antibody can be added to cells in FACS buffer and incubated for 30 min at 4° C. After incubation, the cells are washed to remove unbound antibody. The cells are then dissociated and stained with fluorescent-conjugated secondary antibody for 30 minutes on ice before being analyzed using backman flow cytometry system. The flueorescent intensity and cell binding percentage can be analyzed by Beckman flow cytometric software.

### Drug Moiety

In the ADCs of the present invention, any agent that exerts a therapeutic effect on cancer cells or activated immune cells can be used as the warhead conjugated to an anti-target antigen antibody. Useful classes of cytotoxic or immunosuppressive agents include, for example, antitubulin agents (e.g., auristatins and maytansinoids), DNA minor groove binders, DNA replication inhibitors, alkylating agents (e.g., platinum complexes such as cis-platin, mono(platinum), bis(platinum) and tri-nuclear platinum complexes and- carboplatin), anthracyclines, antibiotics, antifolates, antimetabolites, chemotherapy sensitizers, duocarmycins, etoposides, fluorinated pyrimidines, ionophores, lexitropsins, nitrosoureas, platinols, pre-forming compounds, purine antimetabolites, puromycins, radiation sensitizers, steroids, taxanes, topoisomerase inhibitors, RNA polymerase inhibitors (e.g., amatoxins such as α-amanitin), kinase inhibitors, vinca alkaloids, oligonucleotides, or the like.

Individual cytotoxic or immunosuppressive agents include, for example, an androgen, anthramycin (AMC), asparaginase, 5-azacytidine, azathioprine, bleomycin, busulfan, buthionine sulfoximine, camptothecin, carboplatin, carmustine (BSNU), CC-1065, chlorambucil, cisplatin, colchicine, cyclophosphamide, cytarabine, cytidine arabinoside, cytochalasin B, dacarbazine, dactinomycin (formerly actinomycin), daunorubicin, decarbazine, docetaxel, doxorubicin, an estrogen, 5-fluordeoxyuridine, 5-fluorouracil, gramicidin D, hydroxyurea, idarubicin, ifosfamide, irinotecan, lomustine (CCNU), mechlorethamine, melphalan, 6-mercaptopurine, methotrexate, mithramycin, mitomycin C, mitoxantrone, nitroimidazole, paclitaxel, plicamycin, procarbizine, streptozotocin, tenoposide, 6-thioguanine, thioTEPA, topotecan, vinblastine, vincristine, vinorelbine, VP-16 and VM-26.

In various embodiments, the therapeutic drug moiety is a cytotoxic agent. Suitable cytotoxic agents include, for example, dolastatins (e.g., auristatin E, AFP, MMAF, MMAE), DNA minor groove binders (e.g., enediynes and lexitropsins), duocarmycins, taxanes (e.g., paclitaxel and docetaxel), puromycins, vinca alkaloids, CC-1065, SN-38, topotecan, morpholino-doxorubicin, rhizoxin, cyanomorpholino-doxorubicin, echinomycin, combretastatin, netropsin, epothilone A and B, estramustine, cryptophysins, cemadotin, maytansinoids, discodermolide, eleutherobin, α-amanitin, and mitoxantrone. In various embodiments, the cytotoxic agent is a conventional chemotherapeutic such as, for example, doxorubicin, paclitaxel, melphalan, vinca alkaloids, methotrexate, mitomycin C or etoposide. In addition, potent agents such as CC-1065 analogues, calicheamicin, maytansine, analogues of dolastatin 10, rhizoxin, and palytoxin can be used in the ADCs of the present invention.

The maytansinoid emtansine (also called mertansine, DM1) is a chemical derivative of maytansine. DM1 binds to the ends of microtubules and thereby inhibits the growth and the shortening of microtubules, leading to suppression of microtubule dynamics. Specifically, DM1 showed high-affinity binding (K_{D}, 0.1 μmol/L) to approximately 37 sites per microtubule. DM1 also binds to high-affinity sites on microtubules 20 times more strongly than vinblastine. The DM1-based ADC— Kadcyla (Trastuzumab emtansine, T-DM1), is formulated by the conjugation of Trastuzumab, an anti-HER2/neu monoclonal antibody with DM1 via a non-cleavable SMCC linker. It was approved by FDA in 2013 for the treatment of HER2 positive breast cancer.

In various embodiments, the drug moiety is DM1 having the general formula (XII):

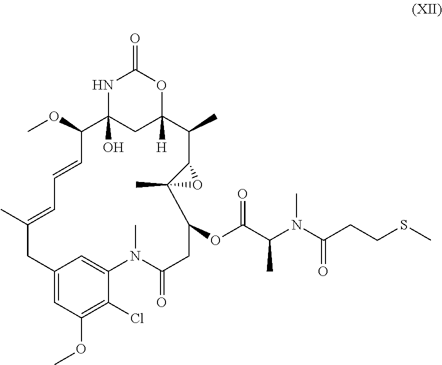

The ADCs of the present invention comprises a linker region between the drug moiety and the anti-target antigen antibody or derivative thereof. “Linker”, “Linker Unit”, “L”, “L²” or “link” means, in the present invention, a chemical moiety comprising a covalent bond or a chain of atoms that covalently attaches an antibody to at least one drug. The linker may be a “non-cleavable” or “cleavable”.

Linkers may be made using a variety of bifunctional protein modifying agents such as N-succinimidyl-3-(2-pyridyldithio) propionate (SPDP), succinimidyl-4-(N-maleimidomethyl)cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-alkyne compounds, bis-diazonium derivatives (such as bis-(p-diazoniumbenzoyl)-ethylenediamine), diisocyanates (such as toluene 2,6-diisocyanate), bis-maleimide compounds, and bis-active fluorine compounds (such as 1,5-difluoro-2,4-dinitrobenzene). Carbon-14-labeled 1-isothiocyanatobenzyl-3-methyldiethylene triaminepentaacetic acid (MX-DTPA) is an exemplary chelating agent for conjugation of cyctotoxic agents to the addressing system. Other cross-linker reagents may be BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, Ill., U.S.A).

In various embodiments, the ADC comprises a “cleavable linker” facilitating release of the drug moiety in the cell. For example, an acid-labile linker, peptidase-sensitive linker, photolabile linker, or disulfide-containing linker may be used such that cleavage of the linker releases the drug moiety from the antibody in the intracellular environment. In various embodiments, the linker is a peptidyl linker that is cleaved by an intracellular peptidase or protease enzyme, including, but not limited to, a lysosomal or endosomal protease. Cleaving agents can include cathepsins B and D and plasmin, all of which are known to hydrolyze dipeptide drug derivatives resulting in the release of active drug inside target cells. In various embodiments, a peptidyl linker that is cleavable by the thiol-dependent protease cathepsin-B, which is highly expressed in cancerous tissue, can be used (e.g., a linker comprising or being Phe-Leu or Gly-Phe-Leu-Gly (SEQ ID NO: 9)). In various embodiments, the peptidyl linker cleavable by an intracellular protease comprises or is Val-Cit or Phe-Lys. In various embodiments, the cleavable linker is pH-sensitive, i.e., sensitive to hydrolysis at certain pH values. Typically, the pH-sensitive linker is hydrolyzable under acidic conditions. For example, an acid-labile linker that is hydrolyzable in the lysosome (e.g., a hydrazone, semicarbazone, thiosemicarbazone, cis-aconitic amide, orthoester, acetal, ketal, or the like) can be used. Such linkers are relatively stable under neutral pH conditions, such as those in the blood, but are unstable at below pH 5.5 or 5.0, the approximate pH of the lysosome. In certain embodiments, the hydrolyzable linker is a thioether linker (such as, e.g., a thioether attached to the drug via an acylhydrazone bond). In various embodiments, the linker is cleavable under reducing conditions (e.g., a disulfide linker). A variety of disulfide linkers are known in the art, including, for example, those that can be formed using SATA (N-succinimidyl-S-acetylthioacetate), SPDP (N-succinimidyl-3-(2-pyridyldithio)propionate), SPDB (N-succinimidyl-3-(2-pyridyldithio)butyrate) and SMPT (N-succinimidyl-oxycarbonyl-alpha-methyl-alpha-(2-pyridyl-dithio)toluene).

In various embodiments, the linker unit has the following general formula (XIII):

L¹L^{2a}A′L³-C(═O)— (XIII)

The antibody is connected to the terminal of L¹ and the antitumor toxin is connected to carboyl group of the -L³-C(═O)— moiety.

L¹ represents -(Succinimid-3-yl-N)—(CH2)ₙ₁-C(═O)—. The “-(Succinimid-3-yl-N)” has a structure represented by the following formula:

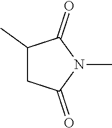

Position 3 of the above partial structure is a connecting position to the antibody. The bond to the antibody at position 3 is characterized by bonding with thioether formation. The nitrogen atom at the position 1 of the structure moiety is connected to the carbon atom of methylene which is present within the linker including the structure.

L^{2a} represents —NH—(CH2-CH2-O)ₙ2-CH2-. A′ represents 1,2,3-triazole. L³ represents —(CH₂)n³-, n=2-5, or —(CH₂)₂—O—(CH2)n⁴-, n=2, m=1-2. Maytansinol was coupled with N-methyl-N-(6-azido-L³-C(═O)-L-alanine to form azido-L³-C(═O)-D. L-D was prepared by click reaction of azido-L³-C(═O)-D with L¹L^{2a}-alkyne.

Techniques for conjugating therapeutic agents to proteins, and in particular to antibodies, are well-known. (See, e.g., Arnon et al., “Monoclonal Antibodies For Immunotargeting Of Drugs In Cancer Therapy,” in Monoclonal Antibodies And Cancer Therapy (Reisfeld et al eds., Alan R. Liss, Inc., 1985); Hellstrom et al., “Antibodies For Drug Delivery,” in Controlled Drug Delivery (Robinson et al. eds., Marcel Dekker, Inc., 2nd ed. 1987); Thorpe, “Antibody Carriers Of Cytotoxic Agents In Cancer Therapy: A Review,” in Monoclonal Antibodies '84: Biological And Clinical Applications (Pinchera et al. eds., 1985); “Analysis, Results, and Future Prospective of the Therapeutic Use of Radiolabeled Antibody In Cancer Therapy,” in Monoclonal Antibodies For Cancer Detection And Therapy (Baldwin et al. eds., Academic Press, 1985); and Thorpe et al., 1982, Immunol. Rev. 62:119-58. See also, e.g., PCT publication WO 89/12624.)

Linkers in ADCs may have significant impacts on the biological activities. For example, in vivo studies demonstrated that the peptide-linked conjugates induced regressions and cures of established tumor xenografts with therapeutic indices as high as 60-fold. These conjugates illustrate the importance of linker technology, drug potency and conjugation method in developing safe and efficacious ADCs for cancer therapy.

Some embodiments of the invention relate to DM1s linked to antibodies via a non-cleavable linker. In this example, antibody was reduced by adding antibody to TCEP (Tris (2-carboxyethyl) phosphine) dissolved in a pH-adjusted PBS EDTA buffer. The antibody/TCEP solution was incubated at 37° C. for 2-3 hrs. The reduced antibodies were buffer exchanged into conjugation reaction buffer. The BI-P203 payload dissolved in DMSO was added to a solution of reduced monoclonal antibody at payload/antibody ratio of 7-30:1 in order to achieve different drug to antibody ratios (DARs). The payload/antibody solution was incubated for 1-2 hr at 20° C. while the reduced antibody was conjugated to BI-P203 payload via the maleimide group. After conjugation was completed, the reaction mixture was desalted and concentrated to yield anti-Trop-2-DM1 ADCs. The biochemical properties of the resulting ADCs were characterized using size-exclusion chromatography high pressure liquid chromatography (SEC-H PLC) to determine purity and aggregation content, and by using hydrophobic interaction chromatography HPLC (HIC-HPLC) to confirm drug loading (DAR). The final ADC products are comprised of four or six or seven DM1-linker molecules. The cysteine conjugation method used in the conjugation process produces more homogenous ADCs compared to lysine conjugation method.

In ADCs, high drug loading, e.g. drug ratio >5, may cause aggregation, insolubility, toxicity, or loss of cellular permeability of certain antibody-drug conjugates. Typically, drug moieties conjugated to an antibody during a conjugation reaction are less than the theoretical maximum. The drug loading also referred as the Drug-Antibody ratio (DAR) is the average number of drugs per antibody. In the case of antibody IgG1 and IgG4 isotypes, where the drugs are bound to cysteines after partial antibody reduction, drug loading may range from 1 to 8 drugs (D) per antibody, i.e. where 2, 4, 5, 6, and 8 drug moieties are covalently attached to the antibody. In the case of an antibody IgG2 isotype, where the drugs are bound to cysteines after partial antibody reduction, drug loading may range from 1 to 12 drugs (D) per antibody, i.e. where 2, 4, 6, 8, 10, and 12 drug moieties are covalently attached to the antibody. Compositions of ADCs include collections of cell binding agents, e.g. antibodies, conjugated with a range of drugs, from 1 to 8 or 1 to 12. The average number of drugs per antibody in preparations of ADCs from conjugation reactions may be characterized by conventional means such as UV, reverse phase HPLC, HIC, mass spectrometry, ELISA assay, and electrophoresis. The BI-P203 containing ADCs of the present invention has much higher solubility compared to those ADCs containing SMCC-DM1 or vc-MMAE payload, therefore allow more drugs to be conjugated to the antibody i.e., DAR≥7.

### Pharmaceutical Compositions

In another aspect, the present invention provides a pharmaceutical composition comprising an ADC as described herein, with one or more pharmaceutically acceptable excipient(s). The pharmaceutical compositions and methods of uses described herein also encompass embodiments of combinations (co-administration) with other active agents, as detailed below. The ADCs provided herein can be formulated by a variety of methods apparent to those of skill in the art of pharmaceutical formulation. Such methods may be found, for example, in Remington's Pharmaceutical Sciences, 19th Edition (Mack Publishing Company, 1995). The pharmaceutical compositions are generally formulated as sterile, substantially isotonic and in full compliance with all GMP regulations of the U.S. Food and Drug Administration.

Generally, ADCs of the invention are suitable to be administered as a formulation in association with one or more pharmaceutically acceptable excipient(s), or carriers. Such pharmaceutically acceptable excipients and carriers are well known and understood by those of ordinary skill and have been extensively described (see, e.g., Remington's Pharmaceutical Sciences, 18th Edition, A. R. Gennaro, ed., Mack Publishing Company, 1990). The pharmaceutically acceptable carriers may be included for purposes of modifying, maintaining or preserving, for example, the pH, osmolarity, viscosity, clarity, color, isotonicity, odor, sterility, stability, rate of dissolution or release, adsorption or penetration of the composition. Such pharmaceutical compositions may influence the physical state, stability, rate of in vivo release, and rate of in vivo clearance of the polypeptide. Suitable pharmaceutically acceptable carriers include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates, other organic acids); bulking agents (such as mannitol or glycine), chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides and other carbohydrates (such as glucose, mannose, or dextrins); proteins (such as serum albumin, gelatin or immunoglobulins); coloring; flavoring and diluting agents; emulsifying agents; hydrophilic polymers (such as polyvinylpyrrolidone); low molecular weight polypeptides; salt-forming counter ions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (sucrose or sorbitol); tonicity enhancing agents (such as alkali metal halides (preferably sodium or potassium chloride, mannitol sorbitol); delivery vehicles; diluents; excipients and/or pharmaceutical adjuvants.

The primary vehicle or carrier in a pharmaceutical composition may be either aqueous or non-aqueous in nature. For example, a suitable vehicle or carrier may be water for injection, physiological saline solution or artificial cerebrospinal fluid, possibly supplemented with other materials common in compositions for parenteral administration. Neutral buffered saline or saline mixed with serum albumin are further exemplary vehicles. Other exemplary pharmaceutical compositions comprise Tris buffer of about pH 7.0-8.5, or acetate buffer of about pH 4.0-5.5, which may further include sorbitol or a suitable substitute thereof. In one embodiment of the present invention, compositions may be prepared for storage by mixing the selected composition having the desired degree of purity with optional formulation agents (Remington's Pharmaceutical Sciences, supra) in the form of a lyophilized cake or an aqueous solution. Further, the therapeutic composition may be formulated as a lyophilizate using appropriate excipients such as sucrose. The optimal pharmaceutical composition will be determined by one of ordinary skill in the art depending upon, for example, the intended route of administration, delivery format, and desired dosage.

The pharmaceutical compositions of the invention are typically suitable for parenteral administration. As used herein, “parenteral administration” of a pharmaceutical composition includes any route of administration characterized by physical breaching of a tissue of a patient and administration of the pharmaceutical composition through the breach in the tissue, thus generally resulting in the direct administration into the blood stream, into muscle, or into an internal organ. Parenteral administration thus includes, but is not limited to, administration of a pharmaceutical composition by injection of the composition, by application of the composition through a surgical incision, by application of the composition through a tissue-penetrating non-surgical wound, and the like. In various embodiments, the pharmaceutical composition is formulated for parenteral administration via a route selected from, e.g., subcutaneous injection, intraperitoneal injection, intramuscular injection, intrasternal injection, intravenous injection, intraarterial injection, intrathecal injection, intraventricular injection, intraurethral injection, intracranial injection, intrasynovial injection or via infusions.

When parenteral administration is contemplated, the therapeutic pharmaceutical compositions may be in the form of a pyrogen-free, parenterally acceptable aqueous solution comprising the desired ADC in a pharmaceutically acceptable vehicle. A particularly suitable vehicle for parenteral injection is sterile distilled water in which a polypeptide is formulated as a sterile, isotonic solution, properly preserved. In various embodiments, pharmaceutical formulations suitable for injectable administration may be formulated in aqueous solutions, preferably in physiologically compatible buffers such as Hanks' solution, Ringer's solution, or physiologically buffered saline. Aqueous injection suspensions may contain substances that increase the viscosity of the suspension, such as sodium carboxymethyl cellulose, sorbitol, or dextran. Additionally, suspensions of the active compounds may be prepared as appropriate oily injection suspensions. Optionally, the suspension may also contain suitable stabilizers or agents to increase the solubility of the compounds and allow for the preparation of highly concentrated solutions. Injectable formulations may be prepared, packaged, or sold in unit dosage form, such as in ampoules or in multi-dose containers containing a preservative. Other parentally administrable formulations which are useful include those which comprise the active ingredient in microcrystalline form, or in a liposomal preparation. Formulations for parenteral administration may be formulated to be immediate and/or modified release. Modified release formulations include delayed-, sustained-, pulsed-, controlled-, targeted and programmed release.

Any method for formulating and administering peptides, proteins, antibodies, and immunoconjugates accepted in the art may suitably be employed for administering the ADCs of the present invention.

### Dosing

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. The term “dosage unit form,” as used herein, refers to physically discrete units suited as unitary dosages for the subjects to be treated; each unit contains a predetermined quantity of active compound calculated to produce the desired therapeutic effect in association with the required pharmaceutical carrier.

The precise dose of ADC to be employed in the methods of the present invention will depend on the route of administration, and the seriousness of the disease or disorder, and should be decided according to the judgment of the practitioner and each subject's circumstances. It is to be noted that dosage values may include single or multiple doses, and that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition. Further, the dosage regimen with the compositions of this disclosure may be based on a variety of factors, including the type of disease, the age, weight, sex, medical condition of the subject, the severity of the condition, the route of administration, and the particular antibody employed. Thus, the dosage regimen can vary widely, but can be determined routinely using standard methods. For example, doses may be adjusted based on pharmacokinetic or pharmacodynamic parameters, which may include clinical effects such as toxic effects and/or laboratory values. Thus, the present invention encompasses intra-subject dose-escalation as determined by the skilled artisan. Determining appropriate dosages and regimens are well-known in the relevant art and would be understood to be encompassed by the skilled artisan once provided the teachings disclosed herein.

For administration to human subjects, the total monthly dose of the ADCs of the invention can be in the range of 0.002-500 mg per patient, 0.002-400 mg per patient, 0.002-300 mg per patient, 0.002-200 mg per patient, 0.002-100 mg per patient, 0.002-50 mg per patient, 0.006-500 mg per patient, 0.006-400 mg per patient, 0.006-300 mg per patient, 0.006-200 mg per patient, 0.006-100 mg per patient, 0.006-50 mg per patient, 0.02-500 mg per patient, 0.02-400 mg per patient, 0.02-300 mg per patient, 0.02-200 mg per patient, 0.02-100 mg per patient, 0.02-50 mg per patient, 0.06-500 mg per patient, 0.06-400 mg per patient, 0.06-300 mg per patient, 0.06-200 mg per patient, 0.06-100 mg per patient, 0.06-50 mg per patient, 0.2-500 mg per patient, 0.2-400 mg per patient, 0.2-300 mg per patient, 0.2-200 mg per patient, 0.2-100 mg per patient, 0.2-50 mg per patient, 0.6-500 mg per patient, 0.6-400 mg per patient, 0.6-300 mg per patient, 0.6-200 mg per patient, 0.6-100 mg per patient, or 0.6-50 mg per patient, 2-500 mg per patient, 2-400 mg per patient, 2-300 mg per patient, 2-200 mg per patient, 2-100 mg per patient, 2-50 mg per patient, 6-500 mg per patient, 6-400 mg per patient, 6-300 mg per patient, 6-200 mg per patient, 6-100 mg per patient, or 6-50 mg per patient, depending, of course, on the mode of administration. The total monthly dose can be administered in single or divided doses and can, at the physician's discretion, fall outside of the typical ranges given herein.

An exemplary, non-limiting weekly dosing range for a therapeutically effective amount of the ADCs of the invention can be about 0.0001 to about 0.9 mg/kg, about 0.0001 to about 0.8 mg/kg, about 0.0001 to about 0.7 mg/kg, about 0.0001 to about 0.6 mg/kg, about 0.0001 to about 0.5 mg/kg, about 0.0001 to about 0.4 mg/kg, about 0.0001 to about 0.3 mg/kg, about 0.0001 to about 0.2 mg/kg, about 0.0001 to about 0.1 mg/kg, about 0.0003 to about 0.9 mg/kg, about 0.0003 to about 0.8 mg/kg, about 0.0003 to about 0.7 mg/kg, about 0.0003 to about 0.6 mg/kg, about 0.0003 to about 0.5 mg/kg, about 0.0003 to about 0.4 mg/kg, about 0.0003 to about 0.3 mg/kg, about 0.0003 to about 0.2 mg/kg, about 0.0003 to about 0.1 mg/kg, about 0.001 to about 0.9 mg/kg, about 0.001 to about 0.8 mg/kg, about 0.001 to about 0.7 mg/kg, about 0.001 to about 0.6 mg/kg, about 0.001 to about 0.5 mg/kg, about 0.001 to about 0.4 mg/kg, about 0.001 to about 0.3 mg/kg, about 0.001 to about 0.2 mg/kg, about 0.0001 to about 0.1 mg/kg, about 0.003 to about 0.9 mg/kg, about 0.003 to about 0.8 mg/kg, about 0.003 to about 0.7 mg/kg, about 0.003 to about 0.6 mg/kg, about 0.003 to about 0.5 mg/kg, about 0.003 to about 0.4 mg/kg, about 0.003 to about 0.3 mg/kg, about 0.003 to about 0.2 mg/kg, about 0.003 to about 0.1 mg/kg, about 0.01 to about 0.9 mg/kg, about 0.01 to about 0.8 mg/kg, about 0.01 to about 0.7 mg/kg, about 0.01 to about 0.6 mg/kg, about 0.01 to about 0.5 mg/kg, about 0.01 to about 0.4 mg/kg, about 0.01 to about 0.3 mg/kg, about 0.01 to about 0.2 mg/kg, about 0.01 to about 0.1 mg/kg, about 0.03 to about 0.9 mg/kg, about 0.03 to about 0.8 mg/kg, about 0.03 to about 0.7 mg/kg, about 0.03 to about 0.6 mg/kg, about 0.03 to about 0.5 mg/kg, about 0.03 to about 0.4 mg/kg, about 0.03 to about 0.3 mg/kg, about 0.03 to about 0.2 mg/kg, about 0.03 to about 0.1 mg/kg, about 0.1 to about 0.9 mg/kg, about 0.1 to about 0.8 mg/kg, about 0.1 to about 0.7 mg/kg, about 0.1 to about 0.6 mg/kg, about 0.1 to about 0.5 mg/kg, about 0.1 to about 0.4 mg/kg, about 0.1 to about 0.3 mg/kg, about 0.1 to about 0.2 mg/kg, about 0.1 to about 0.1 mg/kg, about 0.3 to about 0.9 mg/kg, about 0.3 to about 0.8 mg/kg, about 0.3 to about 0.7 mg/kg, about 0.3 to about 0.6 mg/kg, about 0.3 to about 0.5 mg/kg, about 0.3 to about 0.4 mg/kg, about 0.3 to about 0.3 mg/kg, about 0.3 to about 0.2 mg/kg, about 0.3 to about 0.1 mg/kg.

In various embodiments, single or multiple administrations of the pharmaceutical compositions are administered depending on the dosage and frequency as required and tolerated by the patient. In any event, the composition should provide a sufficient quantity of at least one of the ADCs disclosed herein to effectively treat the patient. The dosage can be administered once but may be applied periodically until either a therapeutic result is achieved or until side effects warrant discontinuation of therapy.

The dosing frequency of the administration of the ADC pharmaceutical composition depends on the nature of the therapy and the particular disease being treated. The patient can be treated at regular intervals, such as weekly or monthly, until a desired therapeutic result is achieved, or treated with a loading dose followed by maintenance dose at regular intervals. Exemplary dosing frequencies include, but are not limited to: once weekly without break; once weekly, every other week; once every 2 weeks; once every 3 weeks; weakly without break for 2 weeks, twice weekly without break for 2 weeks, twice weekly without break for 3 weeks, twice weekly without break for 4 weeks, twice weekly without break for 5 weeks, twice weekly without break for 6 weeks, twice weekly without break for 7 weeks, twice weekly without break for 8 weeks, monthly; once every other month; once every three months; once every four months; once every five months; or once every six months, or yearly.

Toxicity and therapeutic index of the pharmaceutical compositions of the invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effective dose is the therapeutic index and it can be expressed as the ratio LD₅₀/ED₅₀. Compositions that exhibit large therapeutic indices are generally preferred.

### Therapeutic Methods of Use

In one aspect, the present invention relates to a method of treating a proliferative disease (such as cancer) in an individual, comprising administering to the individual a therapeutically effective amount of an ADC. Importantly, the ADCs and methods described herein can be used to effectively treat cancers, including recurrent, resistant, or refractory cancers, at surprisingly low doses.

In various embodiments, the methods of the present invention are useful in treating certain cellular proliferative diseases. Such diseases include, but are not limited to, the following: a) proliferative diseases of the breast, which include, but are not limited to, invasive ductal carcinoma, invasive lobular carcinoma, ductal carcinoma, lobular carcinoma in situ and metastatic breast cancer; b) proliferative diseases of lymphocytic cells, which include, but are not limited to, various T cell and B cell lymphomas, non-Hodgkins lymphoma, cutaneous T cell lymphoma, Hodgkins disease, and lymphoma of the central nervous system; (c) multiple myeloma, chronic neutrophilic leukemia, chronic eosinophilic leukemia/hypereosinophilic syndrome, chronic idiopathic myelofibrosis, polycythemia vera, essential thrombocythemia, chronic myelomonocytic leukemia, atypical chronic myelogenous leukemia, juvenile myelomonocytic leukemia, refractory anemia with ringed sideroblasts and without ringed sideroblasts, refractory cytopenia (myelodysplastic syndrome) with multilineage dysplasia, refractory anemia (myelodysplastic syndrome) with excess blasts, 5q-syndrome, myelodysplastic syndrome with t(9;12)(q22;p12), and myelogenous leukemia (e.g., Philadelphia chromosome positive (t(9;22)(qq34;q11)); d) proliferative diseases of the skin, which include, but are not limited to, basal cell carcinoma, squamous cell carcinoma, malignant melanoma and Kaposi's sarcoma; e) leukemias, which include, but are not limited to, acute myeloid leukemia, acute lymphoblastic leukemia, chronic lymphocytic leukemia, chronic myelogenous leukemia, and hairy cell leukemia, f) proliferative diseases of the digestive tract, which include, but are not limited to, anal, colon, colorectal, esophageal, gallbladder, stomach (gastric), pancreatic cancer, pancreatic cancer-Islet cell, rectal, small-intestine and salivary gland cancers; g) proliferative diseases of the liver, which include, but are not limited to, hepatocellular carcinoma, cholangiocarcinoma, mixed hepatocellular cholangiocarcinoma, primary liver cancer and metastatic liver cancer; h) proliferative diseases of the male reproductive organs, which include, but are not limited to, prostate cancer, testicular cancer and penile cancer; i) proliferative diseases of the female reproductive organs, which include, but are not limited to, uterine cancer (endometrial), cervical, ovarian, vaginal, vulval cancers, uterine sarcoma and ovarian germ cell tumor; j) proliferative diseases of the respiratory tract, which include, but are not limited to, small cell and non-small cell lung carcinoma, bronchial adema, pleuropulmonary blastoma and malignant mesothelioma; k) proliferative diseases of the brain, which include, but are not limited to, brain stem and hyptothalamic glioma, cerebellar and cerebral astrocytoma, medullablastoma, ependymal tumors, oligodendroglial, meningiomas and neuroectodermal and pineal tumors; I) proliferative diseases of the eye, which include, but are not limited to, intraocular melanoma, retinoblastoma, and rhabdomyosarcoma; m) proliferative diseases of the head and neck, which include, but are not limited to, laryngeal, hypopharyngeal, nasopharyngeal, oropharyngeal cancers, and lip and oral cancer, squamous neck cancer, metastatic paranasal sinus cancer; n) proliferative diseases of the thyroid, which include, but are not limited to, thyroid cancer, thymoma, malignant thymoma, medullary thyroid carcinomas, papillary thyroid carcinomas, multiple endocrine neoplasia type 2A (MEN2A), pheochromocytoma, parathyroid adenomas, multiple endocrine neoplasia type 2B (MEN2B), familial medullary thyroid carcinoma (FMTC) and carcinoids; o) proliferative diseases of the urinary tract, which include, but are not limited to, bladder cancer; p) sarcomas, which include, but are not limited to, sarcoma of the soft tissue, osteosarcoma, malignant fibrous histiocytoma, lymphosarcoma, and rhabdomyosarcoma; q) proliferative diseases of the kidneys, which include, but are not limited to, renal cell carcinoma, clear cell carcinoma of the kidney; and renal cell adenocarcinoma; r) precursor B-lymphoblastic leukemia/lymphoma (precursor B-cell acute lymphoblastic leukemia), B-cell chronic lymphocytic leukemia/small lymphocytic lymphoma, B-cell prolymphocytic leukemia, lymphoplasmacytic lymphoma, splenic marginal zone B-cell lymphoma, hairy cell leukemia, plasma cell myeloma/plasmacytoma, extranodal marginal zone B-cell lymphoma of MALT type, nodal marginal zone B-cell lymphoma, follicular lymphoma, mantle-cell lymphoma, diffuse large B-cell lymphoma, mediastinal large B-cell lymphoma, primary effusion lymphoma and Burkitt's lymphoma/Burkitt cell leukemia; (s) precursor T-lymphoblastic lymphoma/leukemia (precursor T-cell acute lymphoblastic leukemia), T-cell prolymphocytic leukemia, T-cell granular lymphocytic leukemia, aggressive NK-cell leukemia, adult T-cell lymphoma/leukemia (HTLV-1), extranodal NK/T-cell lymphoma, nasal type, enteropathy-type T-cell lymphoma, hepatosplenic gamma-delta T-cell lymphoma, subcutaneous panniculitis-like T-cell lymphoma, mycosis fungoides/Sezary syndrome, anaplastic large-cell lymphoma, T/null cell, primary cutaneous type, peripheral T-cell lymphoma, not otherwise characterized, angioimmunoblastic T-cell lymphoma, anaplastic large-cell lymphoma, T/null cell, and primary systemic type; (t) nodular lymphocyte-predominant Hodgkin's lymphoma, nodular sclerosis Hodgkin's lymphoma (grades 1 and 2), lymphocyte-rich classical Hodgkin's lymphoma, mixed cellularity Hodgkin's lymphoma, and lymphocyte depletion Hodgkin's lymphoma; and (u) AML with t(8;21)(q22;q22), AML1(CBF-alpha)/ETO, acute promyelocytic leukemia (AML with t(15;17)(q22;q11-12) and variants, PML/RAR-alpha), AML with abnormal bone marrow eosinophils (inv(16)(p13q22) or t(16;16)(p13;q11), CBFb/MYH11.times.), and AML with 11q23 (MLL) abnormalities, AML minimally differentiated, AML without maturation, AML with maturation, acute myelomonocytic leukemia, acute monocytic leukemia, acute erythroid leukemia, acute megakaryocytic leukemia, acute basophilic leukemia, and acute panmyelosis with myelofibrosis.

In various embodiments, the proliferative disease is a cancer selected from the group consisting of: B cell lymphoma; a lung cancer (small cell lung cancer and non-small cell lung cancer); a bronchus cancer; a colorectal cancer; a prostate cancer; a breast cancer; a pancreas cancer; a stomach cancer; an ovarian cancer; a urinary bladder cancer; a brain or central nervous system cancer; a peripheral nervous system cancer; an esophageal cancer; a cervical cancer; a melanoma; a uterine or endometrial cancer; a cancer of the oral cavity or pharynx; a liver cancer; a kidney cancer; a biliary tract cancer; a small bowel or appendix cancer; a salivary gland cancer; a thyroid gland cancer; a adrenal gland cancer; an osteosarcoma; a chondrosarcoma; a liposarcoma; a testes cancer; and a malignant fibrous histiocytoma; a skin cancer; a head and neck cancer; lymphomas; sarcomas; multiple myeloma; and leukemias.

In various embodiments, there is provided a method of treating a cancer in an individual, comprising administering to the individual an effective amount of a pharmaceutical composition comprising an ADC, wherein the ADC is administered to the individual at a weekly dosage selected from the group consisting of 0.0001 mg/kg, 0.0003 mg/kg, 0.001 mg/kg, 0.003 mg/kg, 0.01 mg/kg, 0.03 mg/kg, 0.1 mg/kg, 0.2 mg/kg, 0.3 mg/kg, 0.4 mg/kg, 0.5 mg/kg, 0.6 mg/kg, 0.7 mg/kg, 0.8 mg/kg, and 0.9 mg/kg. In various embodiments, the ADC is administered to the individual at a dosage (e.g., at a weekly dosage) included in any of the following ranges: about 0.0001 to about 0.0003 mg/kg, about 0.0003 to about 0.001 mg/kg, about 0.001 to about 0.003 mg/kg, about 0.003 to about 0.01 mg/kg, about 0.01 to about 0.03 mg/kg, about 0.03 to about 0.1 mg/kg, about 0.1 to about 0.3 mg/kg, about 0.3 to about 0.4 mg/kg, about 0.4 to about 0.5 mg/kg, about 0.5 to about 0.6 mg/kg, about 0.6 to about 0.7 mg/kg, about 0.7 to about 0.8 mg/kg, and about 0.8 to about 0.9 mg/kg. In various embodiments, the ADC is administered to the individual at a dosage (e.g., at a weekly dosage) of no greater than about any of: 0.0001 mg/kg, 0.0003 mg/kg, 0.001 mg/kg, 0.003 mg/kg, 0.01 mg/kg, 0.03 mg/kg, 0.1 mg/kg, 0.2 mg/kg, 0.3 mg/kg, 0.4 mg/kg, 0.5 mg/kg, 0.6 mg/kg, 0.7 mg/kg, 0.8 mg/kg, and 0.9 mg/kg. In various embodiments, the cancer expresses the TAA of the ADC of the present invention. In various embodiments, the cancer is a non-TAA expressing cancer in the tumor microenvironment of a TAA expressing cancer.

In various embodiments, the methods may inhibit or prevent the growth or proliferation of TAA expressing or non-TAA expressing tumor cells in an individual, such as for example, by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%. As a result, where the tumor is a solid tumor, the modulation may reduce the size of the solid tumor by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%.

The inhibition of the tumor cell proliferation can be measured by cell-based assays, such as bromodeoxyuridine (BRDU) incorporation (Hoshino et al., Int. J. Cancer 38, 369, 1986; Campana et al., J. Immunol. Meth. 107:79, 1988; [³H]-thymidine incorporation (Chen, J., Oncogene 13:1395-403, 1996; Jeoung, J., J. Biol. Chem. 270:18367-73, 1995; the dye Alamar Blue (available from Biosource International) (Voytik-Harbin et al., In Vitro Cell Dev Biol Anim 34:239-46, 1998). The anchorage independent growth of cancer cells is assessed by colony formation assay in soft agar, such as by counting the number of cancer cell colonies formed on top of the soft agar (see Examples and Sambrook et al., Molecular Cloning, Cold Spring Harbor, 1989).

The inhibition of tumor cell growth in a subject may be assessed by monitoring the cancer growth in a subject, for example in an animal model or in human subjects. One exemplary monitoring method is tumorigenicity assays. In one example, a xenograft comprises human cells from a pre-existing tumor or from a tumor cell line. Tumor xenograft assays are known in the art and described herein (see, e.g., Ogawa et al., Oncogene 19:6043-6052, 2000). In another embodiment, tumorigenicity is monitored using the hollow fiber assay, which is described in U.S. Pat. No. 5,698,413, which is incorporated herein by reference in its entirety.

The percentage of the inhibition is calculated by comparing the tumor cell proliferation, anchorage independent growth, or tumor cell growth under modulator treatment with that under negative control condition (typically without modulator treatment). For example, where the number of tumor cells or tumor cell colonies (colony formation assay), or PRDU or [³H]-thymidine incorporation is A (under the treatment of modulators) and C (under negative control condition), the percentage of inhibition would be (C-A)/C×100%.

Examples of tumor cell lines derived from human tumors and available for use in the in vitro and in vivo studies include, but are not limited to, leukemia cell lines (e.g., CCRF-CEM, HL-60(TB), K-562, MOLT-4, RPM1-8226, SR, P388 and P388/ADR, H292, MV-4-11); non-small cell lung cancer cell lines (e.g., A549/ATCC, EKVX, HOP-62, HOP-92, NCI-H226, NCI-H23, NCI-H322M, NCI-H460, NCI-H522 and LXFL 529); small cell lung cancer cell lines (e.g., DMS 114 and SHP-77); colon cancer cell lines (e.g., COLO 205, HCC-2998, HCT-116, HCT-15, HT29, KM12, SW-620, DLD-1 and KM20L2); central nervous system (CNS) cancer cell lines (e.g., SF-268, SF-295, SF-539, SNB-19, SNB-75, U251, SNB-78 and XF 498); melanoma cell lines (e.g., LOX I MVI, MALME-3M, M14, SK-MEL-2, SK-MEL-28, SK-MEL-5, UACC-257, UACC-62, RPMI-7951 and M19-MEL); ovarian cancer cell lines (e.g., IGROV1, OVCAR-3, OVCAR-4, OVCAR-5, OVCAR-8 and SK-OV-3); renal cancer cell lines (e.g., 786-0, A498, ACHN, CAKI-1, RXF 393, SN12C, TK-10, UO-31, RXF-631 and SN12K1); prostate cancer cell lines (e.g., PC-3 and DU-145); breast cancer cell lines (e.g., MCF7, NCl/ADR-RES, MDA-MB-231/ATCC, HS 578T, MDA-MB-435, BT-549, T-47D and MDA-MB-468); and thyroid cancer cell lines (e.g., SK—N—SH).

In another aspect, the present invention relates to a method of activating or stimulating an non-TAA expressing immune cell located in the tumor microenvironment of a TAA expressing tumor, comprising administering to the individual an effective amount of a pharmaceutical composition comprising an ADC; wherein the ADC is administered to the individual at a dosage (e.g., at a weekly dosage) included in any of the following ranges: about 0.0001 to about 0.0003 mg/kg, about 0.0003 to about 0.001 mg/kg, about 0.001 to about 0.003 mg/kg, about 0.003 to about 0.01 mg/kg, about 0.01 to about 0.03 mg/kg, about 0.03 to about 0.1 mg/kg, about 0.1 to about 0.3 mg/kg, about 0.3 to about 0.4 mg/kg, about 0.4 to about 0.5 mg/kg, about 0.5 to about 0.6 mg/kg, about 0.6 to about 0.7 mg/kg, about 0.7 to about 0.8 mg/kg, and about 0.8 to about 0.9 mg/kg. In various embodiments, the ADC is administered to the individual at a weekly dosage selected from the group consisting of about 0.0001 mg/kg, of about 0.0003 mg/kg, of about 0.001 mg/kg, of about 0.003 mg/kg, of about 0.01 mg/kg, of about 0.03 mg/kg, of about 0.1 mg/kg, of about 0.2 mg/kg, of about 0.3 mg/kg, of about 0.4 mg/kg, of about 0.5 mg/kg, of about 0.6 mg/kg, of about 0.7 mg/kg, of about 0.8 mg/kg, and of about 0.9 mg/kg. In various embodiments, the ADC is administered to the individual at a dosage (e.g., at a weekly dosage) of no greater than about any of: 0.0001 mg/kg, 0.0003 mg/kg, 0.001 mg/kg, 0.003 mg/kg, 0.01 mg/kg, 0.03 mg/kg, 0.1 mg/kg, 0.2 mg/kg, 0.3 mg/kg, 0.4 mg/kg, 0.5 mg/kg, 0.6 mg/kg, 0.7 mg/kg, 0.8 mg/kg, and 0.9 mg/kg.

In various embodiments, the methods described herein may be used in combination with other conventional anti-cancer therapeutic approaches directed to treatment or prevention of proliferative disorders, such approaches including, but not limited to chemotherapy, small molecule kinase inhibitor targeted therapy, surgery, radiation therapy, and stem cell transplantation. For example, such methods can be used in prophylactic cancer prevention, prevention of cancer recurrence and metastases after surgery, and as an adjuvant of other conventional cancer therapy. The present invention recognizes that the effectiveness of conventional cancer therapies (e.g., chemotherapy, radiation therapy, phototherapy, immunotherapy, and surgery) can be enhanced through use of the fusion molecules described herein.

A wide array of conventional compounds has been shown to have anti-neoplastic activities. These compounds have been used as pharmaceutical agents in chemotherapy to shrink solid tumors, prevent metastases and further growth, or decrease the number of malignant T-cells in leukemic or bone marrow malignancies. Although chemotherapy has been effective in treating various types of malignancies, many anti-neoplastic compounds induce undesirable side effects. It has been shown that when two or more different treatments are combined, the treatments may work synergistically and allow reduction of dosage of each of the treatments, thereby reducing the detrimental side effects exerted by each compound at higher dosages. In other instances, malignancies that are refractory to a treatment may respond to a combination therapy of two or more different treatments.

When the ADC disclosed herein is administered in combination with another conventional anti-neoplastic agent, either concomitantly or sequentially, such fusion molecule may enhance the therapeutic effect of the anti-neoplastic agent or overcome cellular resistance to such anti-neoplastic agent. This allows decrease of dosage of an anti-neoplastic agent, thereby reducing the undesirable side effects, or restores the effectiveness of an anti-neoplastic agent in resistant T-cells. In various embodiments, a second anti-cancer agent, such as a chemotherapeutic agent, will be administered to the patient. The list of exemplary chemotherapeutic agent includes, but is not limited to, daunorubicin, dactinomycin, doxorubicin, bleomycin, mitomycin, nitrogen mustard, chlorambucil, melphalan, cyclophosphamide, 6-mercaptopurine, 6-thioguanine, bendamustine, cytarabine (CA), 5-fluorouracil (5-FU), floxuridine (5-FUdR), methotrexate (MTX), colchicine, vincristine, vinblastine, etoposide, teniposide, cisplatin, carboplatin, oxaliplatin, pentostatin, cladribine, cytarabine, gemcitabine, pralatrexate, mitoxantrone, diethylstilbestrol (DES), fluradabine, ifosfamide, hydroxyureataxanes (such as paclitaxel and doxetaxel) and/or anthracycline antibiotics, as well as combinations of agents such as, but not limited to, DA-EPOCH, CHOP, CVP or FOLFOX. In various embodiments, the dosages of such chemotherapeutic agents include, but is not limited to, about any of 10 mg/m², 20 mg/m², 30 mg/m², 40 mg/m², 50 mg/m², 60 mg/m², 75 mg/m², 80 mg/m², 90 mg/m², 100 mg/m², 120 mg/m², 150 mg/m², 175 mg/m², 200 mg/m², 210 mg/m², 220 mg/m², 230 mg/m², 240 mg/m², 250 mg/m², 260 mg/m², and 300 mg/m².

### Combination Immunotherapy

In another aspect, the present invention relates to combination therapies designed to treat a proliferative disease (such as cancer) in an individual, comprising administering to the individual: a) a therapeutically effective amount of a ADC, and b) immunotherapy, wherein the combination therapy optionally provides increased effector cell killing of tumor cells, i.e., a synergy exists between the ADC and the immunotherapy when co-administered.

In various embodiments, the proliferative disease is a cancer selected from the group consisting of: B cell lymphoma; a lung cancer (small cell lung cancer and non-small cell lung cancer); a bronchus cancer; a colorectal cancer; a prostate cancer; a breast cancer; a pancreas cancer; a stomach cancer; an ovarian cancer; a urinary bladder cancer; a brain or central nervous system cancer; a peripheral nervous system cancer; an esophageal cancer; a cervical cancer; a melanoma; a uterine or endometrial cancer; a cancer of the oral cavity or pharynx; a liver cancer; a kidney cancer; a biliary tract cancer; a small bowel or appendix cancer; a salivary gland cancer; a thyroid gland cancer; a adrenal gland cancer; an osteosarcoma; a chondrosarcoma; a liposarcoma; a testes cancer; and a malignant fibrous histiocytoma; a skin cancer; a head and neck cancer; lymphomas; sarcomas; multiple myeloma; and leukemias.

In various embodiments, the combination therapy may comprise administering to the subject a therapeutically effective amount of immunotherapy, including, but are not limited to, treatment using depleting antibodies to specific tumor antigens; treatment using antibody-drug conjugates; treatment using agonistic, antagonistic, or blocking antibodies to co-stimulatory or co-inhibitory molecules (immune checkpoints) such as CTLA-4, PD-1, PD-L1, CD40, OX-40, CD137, GITR, LAG3, TIM-3, Siglec 7, Siglec 8, Siglec 9, Siglec 15 and VISTA; treatment using bispecific T cell engaging antibodies (BITE®) such as blinatumomab: treatment involving administration of biological response modifiers such as IL-12, IL-21, GM-CSF, IFN-α, IFN-β and IFN-γ; treatment using therapeutic vaccines such as sipuleucel-T; treatment using dendritic cell vaccines, or tumor antigen peptide vaccines; treatment using chimeric antigen receptor (CAR)-T cells; treatment using CAR-NK cells; treatment using tumor infiltrating lymphocytes (TILs); treatment using adoptively transferred anti-tumor T cells (ex vivo expanded and/or TCR transgenic); treatment using TALL-104 cells; and treatment using immunostimulatory agents such as Toll-like receptor (TLR) agonists CpG and imiquimod; and treatment using vaccine such as BCG.

In various embodiments, there is provided a combination therapy method of treating a proliferative disease in an individual, comprising administering to the individual a) an effective amount of an ADC; and b) immunotherapy; wherein the combination therapy provides increased effector cell killing. In various embodiments, the immunotherapy is treatment using agonistic, antagonistic, or blocking antibodies to co-stimulatory or co-inhibitory molecules. In various embodiments, the immunotherapy is treatment using chimeric antigen receptor (CAR)-T cells. In various embodiments, the immunotherapy is treatment using CAR-NK cells. In various embodiments, the immunotherapy is treatment using bispecific T cell engaging antibodies (BITE®). In various embodiments, the cancer expresses the TAA of the ADC of the present invention. In various embodiments, the cancer is a non-TAA expressing cancer in the tumor microenvironment of a TAA expressing cancer. In various embodiments, the immunotherapy will target a TAA that is different than the TAA targeted by the ADC.

### Kits

In certain embodiments, this invention provides for kits for the treatment of cancer and/or in an adjunct therapy. Kits typically comprise a container containing an ADC of the present invention. The ADC can be present in a pharmacologically acceptable excipient. The kits may optionally include an immunotherapy cancer agent.

In addition, the kits can optionally include instructional materials disclosing means of use of the ADC and/or immunotherapy to treat a cancer. The instructional materials may also, optionally, teach preferred dosages, counter-indications, and the like.

The kits can also include additional components to facilitate the particular application for which the kit is designed. Thus, for example, and additionally comprise means for disinfecting a wound, for reducing pain, for attachment of a dressing, and the like.

While the instructional materials typically comprise written or printed materials, they are not limited to such. Any medium capable of storing such instructions and communicating them to an end user is contemplated by this invention. Such media include but are not limited to electronic storage media (e.g., magnetic discs, tapes, cartridges, chips), optical media (e.g., CD ROM), and the like. Such media may include addresses to internet sites that provide such instructional materials.

The following examples are offered to more fully illustrate the invention but are not construed as limiting the scope thereof.

### Example 1

### Synthesis of Compounds BI-P204

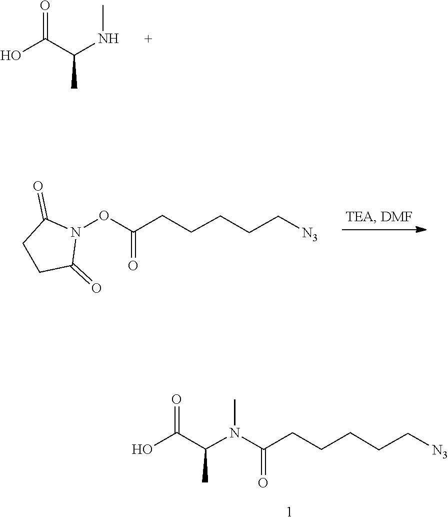

To 6-Azido-hexanoic acid 2,5-dioxo-pyrrolidin-1-yl ester (50 mg, 0.20 mmol) in 2 mL of DMF was added 2-Methylamino-propionic acid (20.3 mg, 0.20 mmol). The mixture was cooled to 0° C. and triethylamine (40 mg, 0.40 mmol) was added. The reaction mixture was stirred at room temperature for 2 hours, then diluted with water, neutralized with 1N HCl to pH˜3, extracted into EtOAc (3×5 mL). The combined organic layer was washed with brine, dried over Na₂SO₄ and concentrated to get compound 1 (30 mg, 63% yield).

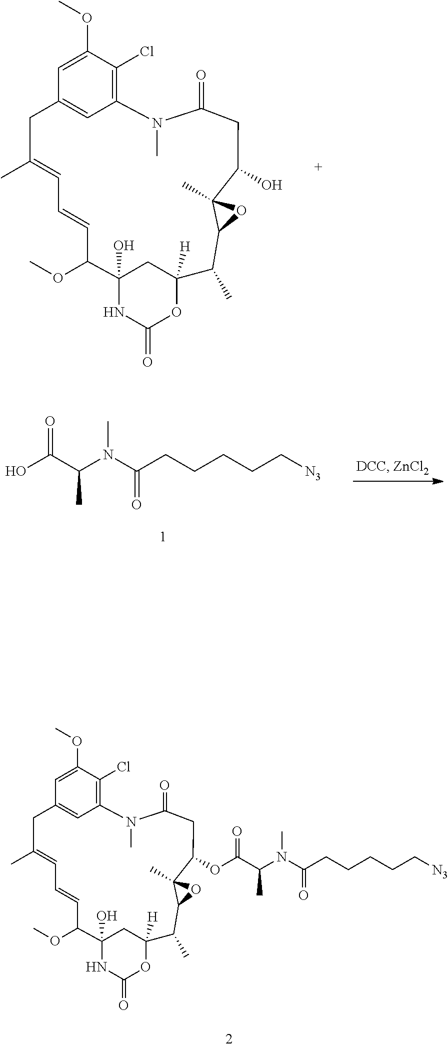 [SMILES: COc1cc2cc(c1Cl)N(C)C(=O)C[C@H](O)[C@@]1(C)O[C@H]1[C@H](C)[C@@H]1C[C@@](O)(NC(=O)O1)C(OC)/C=C/C=C(\C)C2 ; C[C@@H](C(=O)O)N(C)C(=O)CCCCCN=[N+]=[N-] ; COc1cc2cc(c1Cl)N(C)C(=O)C[C@H](OC(=O)[C@H](C)N(C)C(=O)CCCCCN=[N+]=[N-])[C@@]1(C)O[C@H]1[C@H](C)[C@@H]1C[C@@](O)(NC(=O)O1)C(OC)/C=C/C=C(\C)C2]

Maytansinol (10 mg, 0.018 mmol) and compound 1 (21.4 mg, 0.088 mmol) were dissolved in DCM (32 mL) and stirred under argon atmosphere. A solution of DCC (20.1 mg, 0.097 mmol) in DCM (1 mL) was added. After 1 min, a solution of 1M ZnCl₂ in diethyl ether (0.019 mL, 0.019 mmol) was added. The mixture was stirred at room temperature for 4 hours, then ethyl acetate was added (5 mL) and the mixture was filtered. The filtrate was washed with NaHCO₃ and brine. The organic layer was dried over Na₂SO₄ and concentrated. The residue was purified by column chromatography on silica gel (50-100% Ethyl acetate/hexane) to get the desired product compound 2 (5 mg, 36% yield).

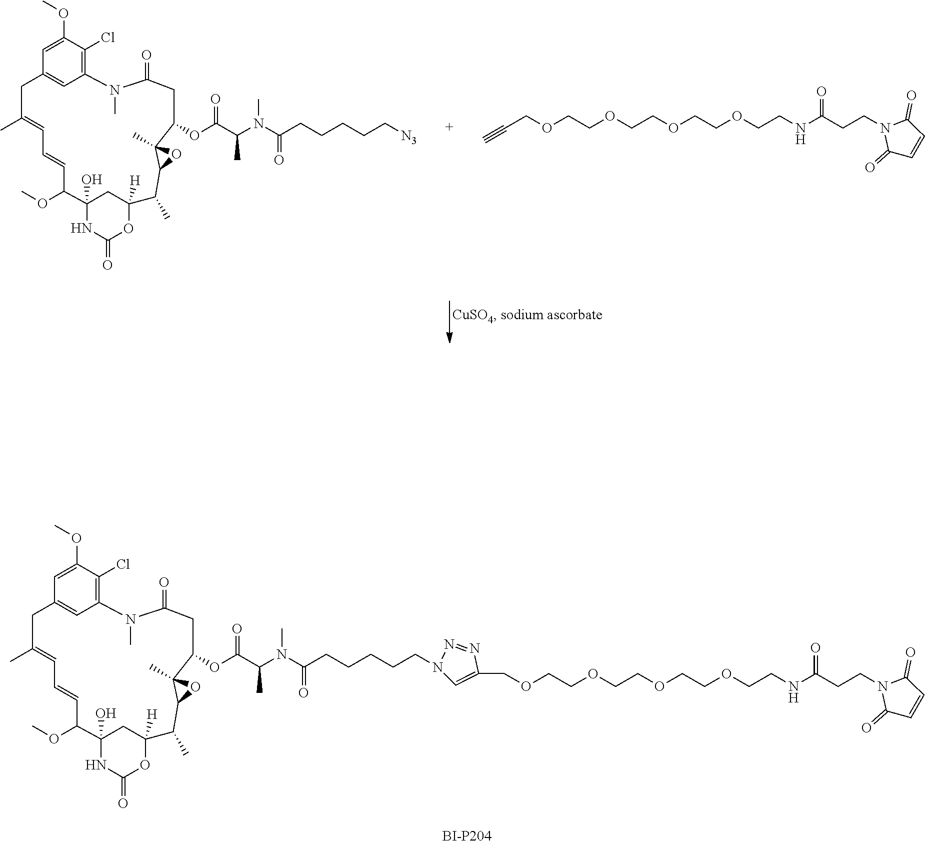 [SMILES: COc1cc2cc(c1Cl)N(C)C(=O)C[C@H](OC(=O)[C@H](C)N(C)C(=O)CCCCCN=[N+]=[N-])[C@@]1(C)O[C@H]1[C@H](C)[C@@H]1C[C@@](O)(NC(=O)O1)C(OC)/C=C/C=C(\C)C2 ; C#CCOCCOCCOCCOCCNC(=O)CCN1C(=O)C=CC1=O ; COc1cc2cc(c1Cl)N(C)C(=O)C[C@H](OC(=O)[C@H](C)N(C)C(=O)CCCCCn1cc(COCCOCCOCCOCCNC(=O)CCN3C(=O)C=CC3=O)nn1)[C@@]1(C)O[C@H]1[C@H](C)[C@@H]1C[C@@](O)(NC(=O)O1)C(OC)/C=C/C=C(\C)C2 ; "BI-P204"]

Compound 2 (16.5 mg, 0.021 mmol) and alkyne-PEG4-Mal (8 mg, 0.021 mmol) were dissolved in DCM (3 mL). To the mixture were added 0.1M of CuSO₄·5H₂O aqueous solution (0.23 mL, 0.023 mmol) and 0.1 M of sodium ascorbate in water (1.26 mL, 0.126 mmol). The final mixture was then vigorously stirred at room temperature overnight, diluted with water, extracted with 10% iso-propanol in CHCl₃. The organic layer was washed with brine, dried over Na₂SO₄ and concentrated. The residue was purified by column chromatography on silica gel (0-7% MeOH/DCM) to get compound BI-P204 (9.3 mg, 38% yield). ESI MS for C₅₆H₇₉ClN₈O₁₇ was calculated as 1171.72 (M) and observed as 1172.96 (M).

### Example 2

### Synthesis of Compounds BI-P203

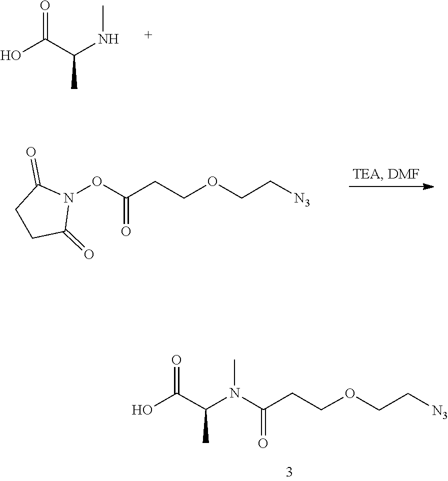

To azido-PEG1-NHS ester (50 mg, 0.20 mmol) in 2 mL of DMF was added 2-Methylamino-propionic acid (20.3 mg, 0.20 mmol). The mixture was cooled to 0° C. and triethylamine (40 mg, 0.40 mmol) was added. The reaction mixture was stirred at room temperature for 2 hours, then diluted with water, neutralized with 1N HCl to pH-3, extracted into EtOAc (3×5 mL). The combined organic layer was washed with brine, dried over Na₂SO₄ and concentrated to get compound 3 (30 mg, 63% yield).

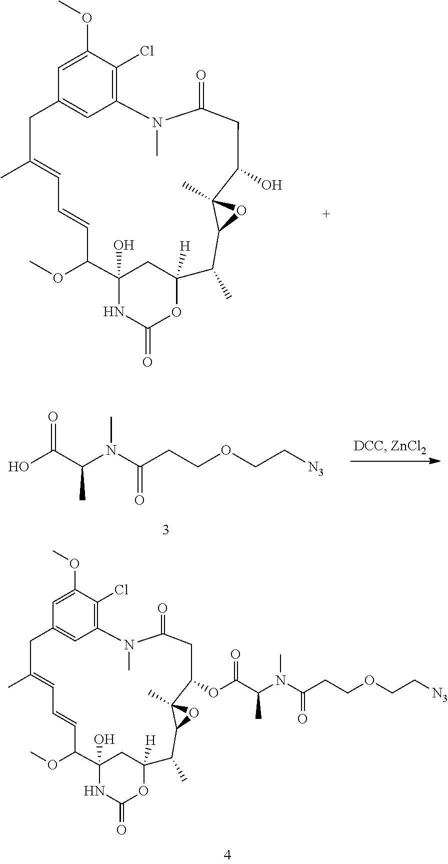 [SMILES: COc1cc2cc(c1Cl)N(C)C(=O)C[C@H](O)[C@@]1(C)O[C@H]1[C@H](C)[C@@H]1C[C@@](O)(NC(=O)O1)C(OC)/C=C/C=C(\C)C2 ; C[C@@H](C(=O)O)N(C)C(=O)CCOCCN=[N+]=[N-] ; COc1cc2cc(c1Cl)N(C)C(=O)C[C@H](OC(=O)[C@H](C)N(C)C(=O)CCOCCN=[N+]=[N-])[C@@]1(C)O[C@H]1[C@H](C)[C@@H]1C[C@@](O)(NC(=O)O1)C(OC)/C=C/C=C(\C)C2]

Maytansinol (20 mg, 0.038 mmol) and compound 3 (42 mg, 0.178 mmol) were dissolved in DCM (32 mL) and stirred under argon atmosphere. A solution of DCC (40 mg, 0.194 mmol) in DCM (1 mL) was added. After 1 min, a solution of 1M ZnCl₂ in diethyl ether (0.038 mL, 0.038 mmol) was added. The mixture was stirred at room temperature for 4 hours, then ethyl acetate was added (5 mL) and the mixture was filtered. The filtrate was washed with NaHCO₃ and brine. The organic layer was dried over Na₂SO₄ and concentrated. The residue was purified by column chromatography on silica gel (50-100% Ethyl acetate/hexane) to get the desired product compound 4 (13 mg, 47% yield).

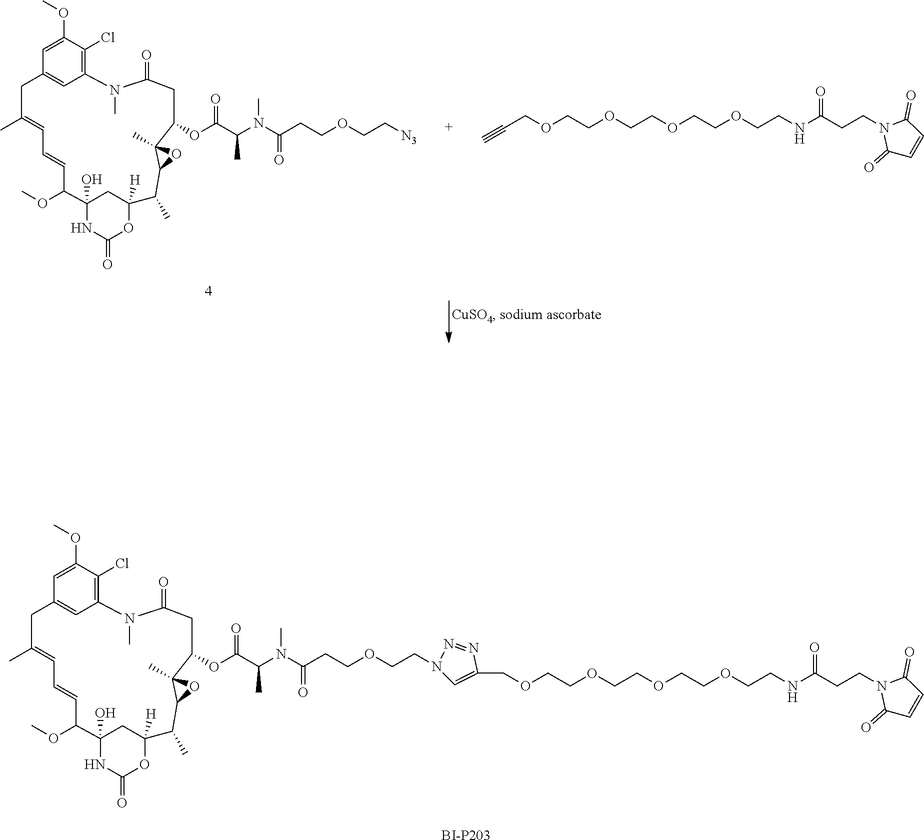

Compound 4 (30 mg, 0.038 mmol) and alkyne-PEG4-Mal (30 mg, 0.078 mmol) were dissolved in DCM (3 mL). To the mixture were added 0.1M of CuSO₄·5H₂O aqueous solution (0.38 mL, 0.038 mmol) and 0.1 M of sodium ascorbate in water (4.8 mL, 0.48 mmol). The final mixture was then vigorously stirred at room temperature overnight, diluted with water, extracted with 10% iso-propanol in CHCl₃. The organic layer was washed with brine, dried over Na₂SO₄ and concentrated. The residue was purified by column chromatography on silica gel (0-7% MeOH/DCM) to get compound BI-P203 (33 mg, 74% yield). ESI MS for C₅₅H₇₇ClN₈O₁₈ was calculated as 1173.70 (M) and observed as 1173.94 (M).

### Example 3

### Synthesis of Compounds BI-P205

To 5-Azido-pentanoic acid 2,5-dioxo-pyrrolidin-1-yl ester (100 mg, 0.42 mmol) in 2 mL of DMF was added 2-Methylamino-propionic acid (45 mg, 0.45 mmol). The mixture was cooled to 0° C. and triethylamine (90 mg, 0.90 mmol) was added. The reaction mixture was stirred at room temperature for 2 hours, then diluted with water, neutralized with 1N HCl to pH˜3, extracted into EtOAc (3×5 mL). The combined organic layer was washed with brine, dried over Na₂SO₄ and concentrated to get compound 5 (70 mg, 77% yield).

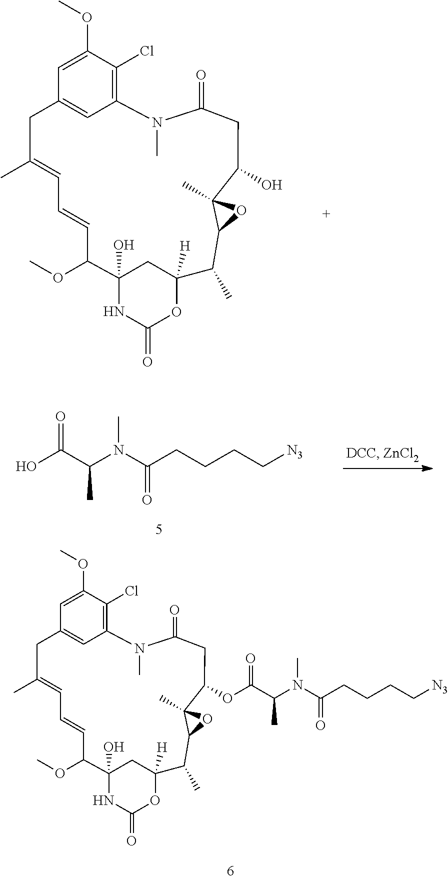 [SMILES: COc1cc2cc(c1Cl)N(C)C(=O)C[C@H](O)[C@@]1(C)O[C@H]1[C@H](C)[C@@H]1C[C@@](O)(NC(=O)O1)C(OC)/C=C/C=C(\C)C2 ; C[C@@H](C(=O)O)N(C)C(=O)CCCCN=[N+]=[N-] ; COc1cc2cc(c1Cl)N(C)C(=O)C[C@H](OC(=O)[C@H](C)N(C)C(=O)CCCCN=[N+]=[N-])[C@@]1(C)O[C@H]1[C@H](C)[C@@H]1C[C@@](O)(NC(=O)O1)C(OC)/C=C/C=C(\C)C2]

Maytansinol (20 mg, 0.038 mmol) and compound 5 (40 mg, 0.176 mmol) were dissolved in DCM (32 mL) and stirred under argon atmosphere. A solution of DCC (40 mg, 0.20 mmol) in DCM (1 mL) was added. After 1 min, a solution of 1M ZnCl₂ in diethyl ether (0.038 mL, 0.038 mmol) was added. The mixture was stirred at room temperature for 4 hours, then ethyl acetate was added (5 mL) and the mixture was filtered. The filtrate was washed with NaHCO₃ and brine. The organic layer was dried over Na₂SO₄ and concentrated. The residue was purified by column chromatography on silica gel (50-100% Ethyl acetate/hexane) to get the desired product compound 6 (12 mg, 41% yield).

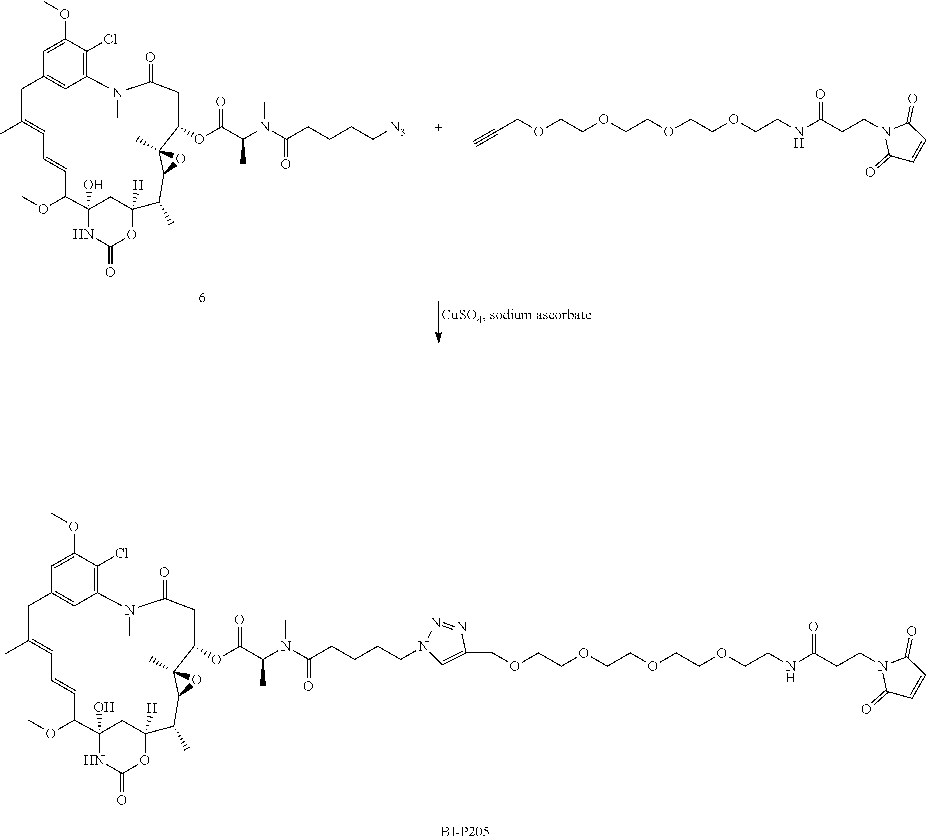 [SMILES: COc1cc2cc(c1Cl)N(C)C(=O)C[C@H](OC(=O)[C@H](C)N(C)C(=O)CCCCN=[N+]=[N-])[C@@]1(C)O[C@H]1[C@H](C)[C@@H]1C[C@@](O)(NC(=O)O1)C(OC)/C=C/C=C(\C)C2 ; C#CCOCCOCCOCCOCCNC(=O)CCN1C(=O)C=CC1=O ; COc1cc2cc(c1Cl)N(C)C(=O)C[C@H](OC(=O)[C@H](C)N(C)C(=O)CCCCn1cc(COCCOCCOCCOCCNC(=O)CCN3C(=O)C=CC3=O)nn1)[C@@]1(C)O[C@H]1[C@H](C)[C@@H]1C[C@@](O)(NC(=O)O1)C(OC)/C=C/C=C(\C)C2 ; "BI-P205"]

Compound 6 (20 mg, 0.026 mmol) and alkyne-PEG4-Mal (10 mg, 0.026 mmol) were dissolved in DCM (3 mL). To the mixture were added 0.1M of CuSO₄·5H₂O aqueous solution (0.27 mL, 0.027 mmol) and 0.1 M of sodium ascorbate in water (1.56 mL, 0.156 mmol). The final mixture was then vigorously stirred at room temperature overnight, diluted with water, extracted with 10% iso-propanol in CHCl₃. The organic layer was washed with brine, dried over Na₂SO₄ and concentrated. The residue was purified by column chromatography on silica gel (0-7% MeOH/DCM) to get compound BI-205 (12 mg, 42% yield). ESI MS for C₅₃H₇₃ClN₈O₁₆ was calculated 1113.64 (M) and observed as: 1113.89 (M).

Compound BI-P209 were prepared in similar procedures as those of compound BI-P203. Compound BI-P209 ESI MS for C₅₃H₇₃ClN₈O₁₈ was calculated as 1128.48 (M) and observed as 1130.03 (M+1).

### Example 4

### Synthesis of Compounds BI-P206

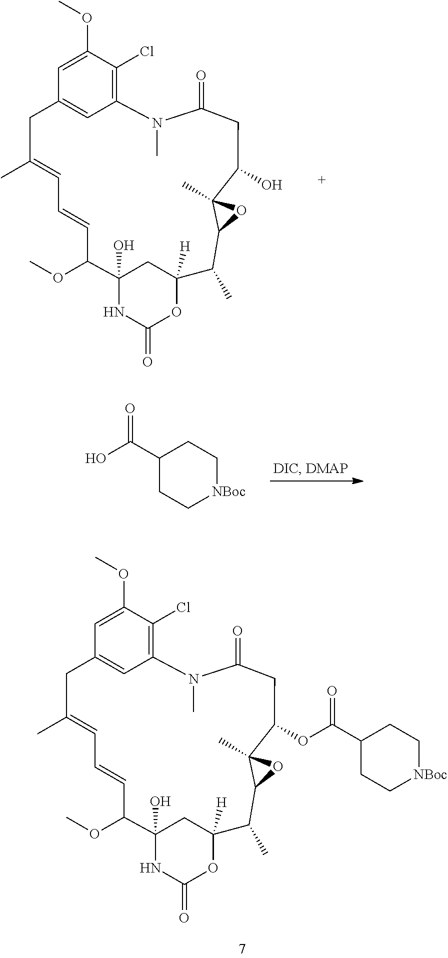 [SMILES: COc1cc2cc(c1Cl)N(C)C(=O)C[C@H](O)[C@@]1(C)O[C@H]1[C@H](C)[C@@H]1C[C@@](O)(NC(=O)O1)C(OC)/C=C/C=C(\C)C2 ; CC(C)(C)OC(=O)N1CCC(C(=O)O)CC1 ; COc1cc2cc(c1Cl)N(C)C(=O)C[C@H](OC(=O)C1CCN(C(=O)OC(C)(C)C)CC1)[C@@]1(C)O[C@H]1[C@H](C)[C@@H]1C[C@@](O)(NC(=O)O1)C(OC)/C=C/C=C(\C)C2]

Maytansinol (56 mg, 0.1 mmol) and N-Boc-piperidine-4-carboxylic acid (207 mg, 0.9 mmol) was dissolved in DCM (3 mL). To the mixture were added DIC (141 μL, 0.9 mmol) and DMAP (9 mg, 0.09 mmol). The mixture was stirred for 3 hrs and the solvent was evaporated to give a residue, which was purified by column chromatography on silica gel (30-100% EtOAc/hexane) to get product 7 (38 mg, 49% yield).

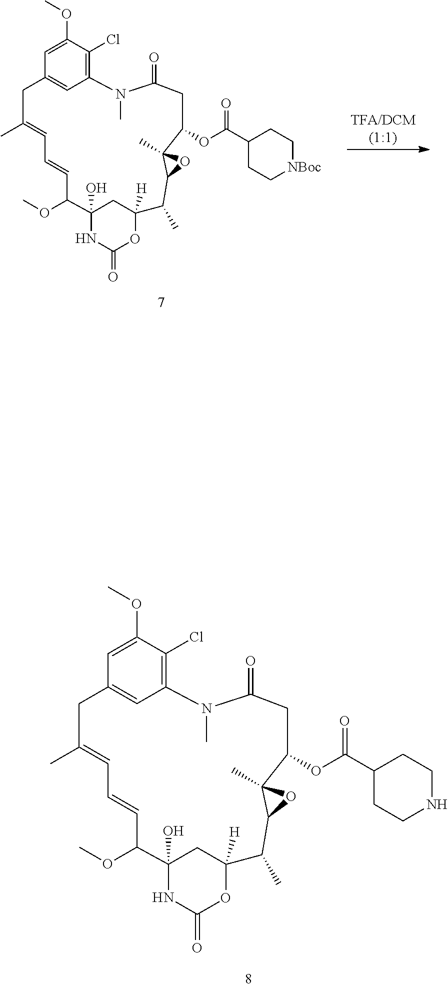 [SMILES: COc1cc2cc(c1Cl)N(C)C(=O)C[C@H](OC(=O)C1CCN(C(=O)OC(C)(C)C)CC1)[C@@]1(C)O[C@H]1[C@H](C)[C@@H]1C[C@@](O)(NC(=O)O1)C(OC)/C=C/C=C(\C)C2 ; COc1cc2cc(c1Cl)N(C)C(=O)C[C@H](OC(=O)C1CCNCC1)[C@@]1(C)O[C@H]1[C@H](C)[C@@H]1C[C@@](O)(NC(=O)O1)C(OC)/C=C/C=C(\C)C2]

Compound 7 (19 mg, 0.0245 mmol) was dissolved in a mixture of DCM and TFA (v/v=1:1, 2 mL). The mixture was stirred for 10 mins and the volatiles were removed under vacuum to give the crude compound 8.

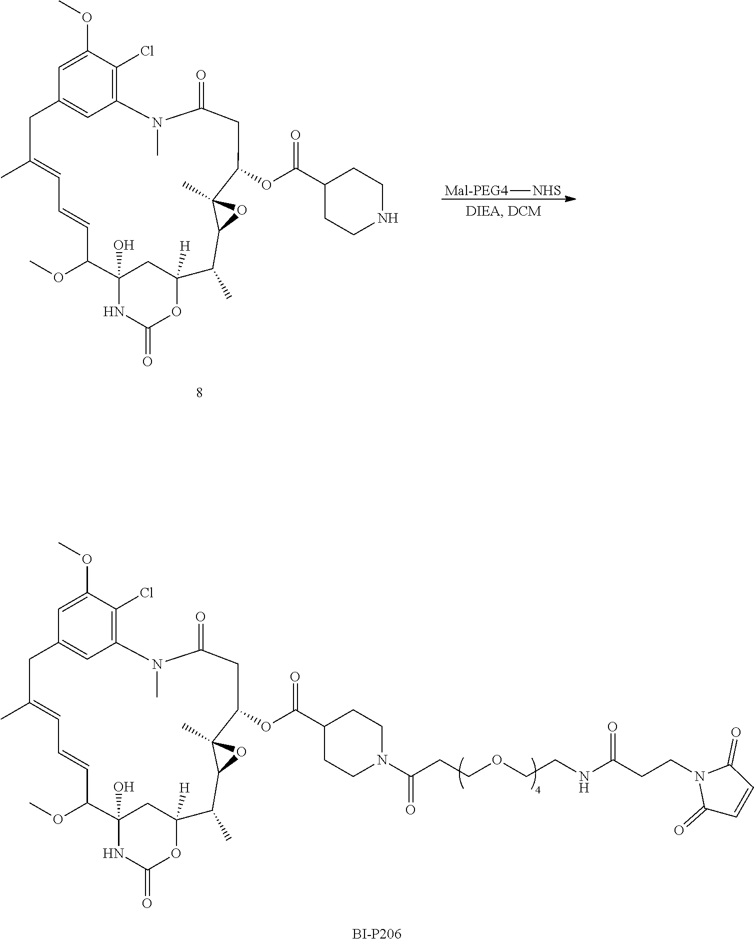

The crude 8 (0.0245 mmol) was dissolved in DCM (1 mL). To the mixture were added DIEA (0.035 mL, 0.2 mmol) and Mal-PEG4-NHS (13 mg, 0.0245 mmol). The mixture was stirred for 30 mins and diluted with DCM (10 mL). Water was added and the organic layer was separated. The organic layer was washed with brine, dried over anhydrous Na₂SO₄ and filtered. The filtrate was evaporated to give a residue, which was purified by column chromatography on silica gel (2-8% MeOH/DCM) to get compound BI-P206 (7.7 mg, 29% yield). ESI MS for C₅₆H₇₉ClN₈O₁₇ was calculated as 1074.60 (M) and observed as 1074.3 (M).

### Example 5

### Synthesis of Compounds BI-P207

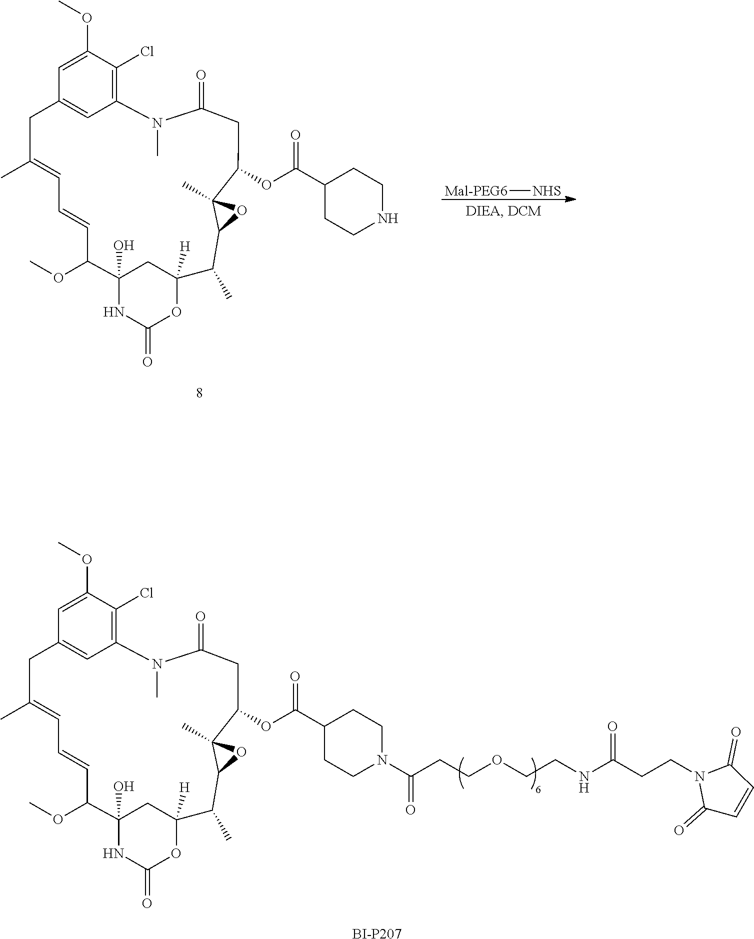

The crude 8 (0.0245 mmol) was dissolved in DCM (1 mL). To the mixture were added DIEA (0.035 mL, 0.2 mmol) and Mal-PEG6-NHS (15 mg, 0.0245 mmol). The mixture was stirred for 30 mins and diluted with DCM (10 mL). Water was added and the organic layer was separated. The organic layer was washed with brine, dried over anhydrous Na₂SO₄ and filtered. The filtrate was evaporated to give a residue, which was purified by column chromatography on silica gel (2-8% MeOH/DCM) to get compound BI-P207 (8.8 mg, 31% yield). ESI MS for C₅₆H₈₀ClN₅O₁₉ was calculated as 1162.71 (M) and observed as 1162.3 (M).

### Example 6

### Synthesis of Compounds BI-P208

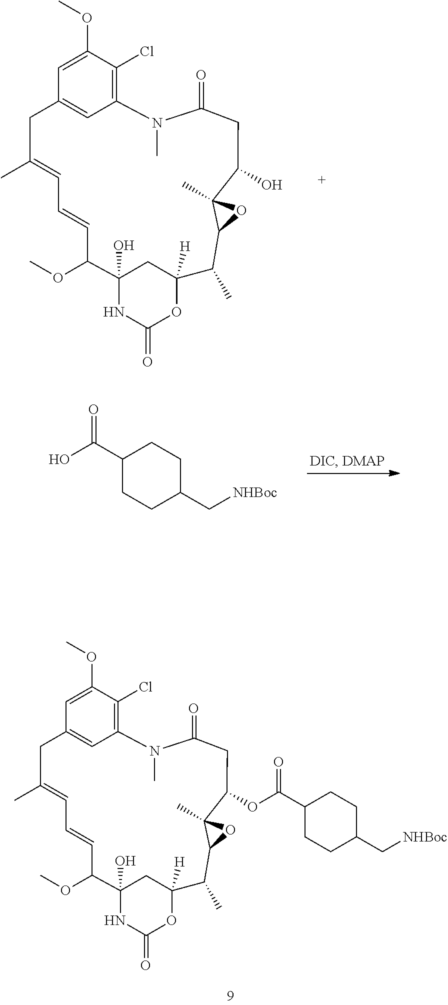 [SMILES: COc1cc2cc(c1Cl)N(C)C(=O)C[C@H](O)[C@@]1(C)O[C@H]1[C@H](C)[C@@H]1C[C@@](O)(NC(=O)O1)C(OC)/C=C/C=C(\C)C2 ; CC(C)(C)OC(=O)NCC1CCC(C(=O)O)CC1 ; COc1cc2cc(c1Cl)N(C)C(=O)C[C@H](OC(=O)C1CCC(CNC(=O)OC(C)(C)C)CC1)[C@@]1(C)O[C@H]1[C@H](C)[C@@H]1C[C@@](O)(NC(=O)O1)C(OC)/C=C/C=C(\C)C2]

Maytansinol (40 mg, 0.071 mmol) and N-Boc-piperidine-4-carboxylic acid (91 mg, 0.3 mmol) was dissolved in DCM (3 mL). To the mixture were added DIC (55 μL, 0.35 mmol) and DMAP (4.3 mg, 0.035 mmol). The mixture was stirred for 3 hrs and the solvent was evaporated to give a residue, which was purified by column chromatography on silica gel (30-100% EtOAc/hexane) to get product 9 (18 mg, 32% yield).

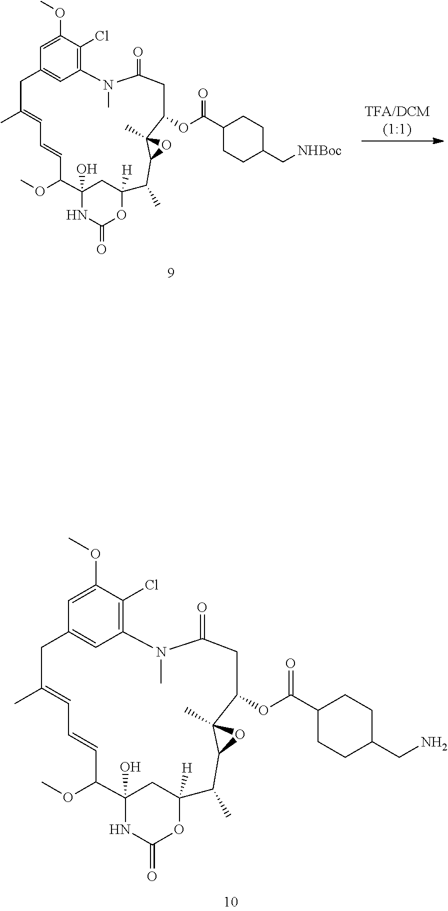

Compound 9 (18 mg, 0.0245 mmol) was dissolved in a mixture of DCM and TFA (v/v=2:1, 0.75 mL). The mixture was stirred for 10 mins and the volatiles were removed under vacuum to give the crude compound 10.

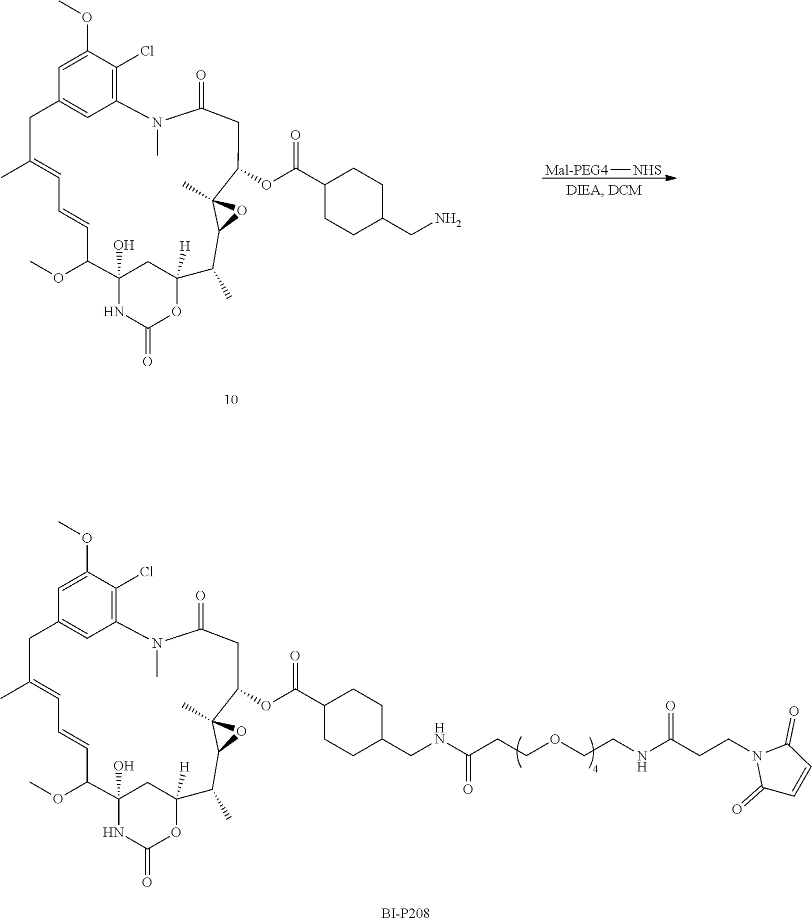

The crude 10 (0.0245 mmol) was dissolved in DCM (1 mL). To the mixture were added DIEA (0.035 mL, 0.2 mmol) and Mal-PEG4-NHS (16 mg, 0.0245 mmol). The mixture was stirred for 30 mins and diluted with DCM (10 mL). Water was added and the organic layer was separated. The organic layer was washed with brine, dried over anhydrous Na₂SO₄ and filtered. The filtrate was evaporated to give a residue, which was purified by column chromatography on silica gel (2-8% MeOH/DCM) to get compound BI-208 (8.8 mg, 31% yield). ESI MS for C₅₄H₇₆ClN₅O₁₇ was calculated as 1102.65 (M) and observed as 1102.3 (M).

Compounds BI-P210 and BI-P211 were prepared in similar procedures as those of compound BI-P206. Compound BI-P210 ESI MS for C₅₄H₇₀ClN₅O₁₇ was calculated as 1096.61 (M) and observed as 1096.8 (M). Compound BI-P211 ESI MS for C₅₀H₆₈ClN₅O₁₇ was calculated as 1046.55 (M) and observed as 1046.8 (M).

### Example 7

### Characterization of DM1 and Maytansinoid Derivatives Payloads

The lipophilicity and aggregation rate of the ADCs of the present invention were characterized. The most commonly used measure of lipophilicity is LogP, which is the partition coefficient of a molecule between aqueous and lipophilic phases, usually octanol and water. LogP is used to predict solubility and permeability that has become a surrogate for drug-likeness. In this experiment, the LogP of anti-Trop-2 BI-P203 and anti-Trop-2-SMCC-DM1 was calculated using ChemDraw software. Size exclusion chromatography (SEC) was used to characterize the ADC monomer and their aggregates. The % aggregation was the percentage of the higher molecular weight species (MW higher than the antibody monomer) determined by the area of peak. The LogP, DAR, and aggregation data are summarized in Table 2. Compared to SMCC-DM1 payload, BI-P203 payload showed increased solubility with decreased aggregation rate. Even with a high DAR of 7, the aggregation rate of anti-Trop-2-BI-P203 was still lower than that of anti-Trop-2-SMCC-DM1 with DAR of 4.

TABLE 2

ADC Characterization

| | | Conjugation | | Aggregation |
|---|---|---|---|---|
| Payload | Log P | method | DAR | rate (%) |
| SMCC-DM1 | 3.904 | Lysine | 3.9 | 3.6 |
| BI-P203 | 0.927 | Cysteine | 3.5 | 0.33 |
| | | | 6.2 | 1.0 |
| | | | 7.0 | 2.0 |
| BI-P204 | 2.081 | Cysteine | 2.8 | 0.65 |
| BI-P205 | 1.674 | Cysteine | 3.6 | 0.57 |
| | | | 7.1 | 3.89 |
| BI-P206 | 1.662 | Cysteine | 3.6 | 0.76 |
| BI-P207 | 1.695 | Cysteine | 3.4 | 1.02 |
| BI-P208 | 2.346 | Cysteine | 3.0 | 2.04 |
| | | | 6.7 | 9.46 |
| | | | 7.3 | 6.02 |
| BI-P209 | 0.52 | Cysteine | 6.2 | 4.96 |

### Example 8

### Generation of Anti-Trop-2-ADCs

Humanized anti-Trop-2 antibody was conjugated to DM1 derivatives to form ADCs and evaluated for their ability to inhibit the growth of multiple cancer cell lines expressing different levels of Trop-2. DM1 is a chemical derivative of maytansine; it inhibits cell division by suppressing microtubule dynamics. DM1 has been shown to be useful payloads for ADCs.

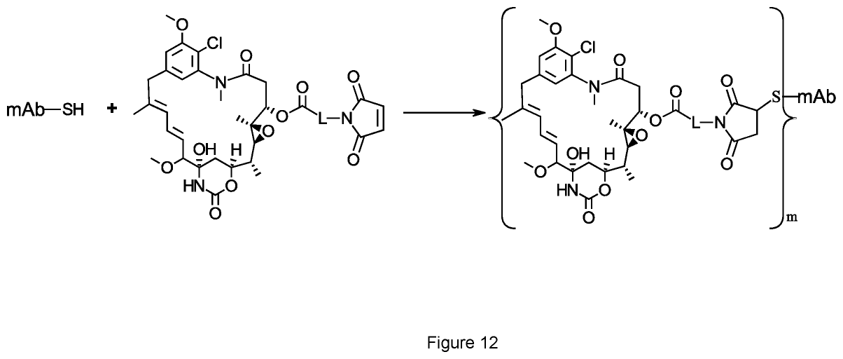

Some embodiments of the invention relate to DM1s linked to antibodies via a non-cleavable linker. In this example, anti-Trop-2 antibody was reduced by adding antibody to TCEP (Tris (2-carboxyethyl) phosphine) dissolved in a pH-adjusted PBS EDTA buffer. The antibody/TCEP solution was incubated at 37° C. for 2-3 hrs. The reduced antibodies were buffer exchanged into conjugation reaction buffer. The BI-P203 payload dissolved in DMSO was added to a solution of reduced anti-Trop-2 monoclonal antibody at payload/antibody ratio of 7-30:1 in order to achieve different drug to antibody ratios (DARs). The payload/antibody solution was incubated for 1-2 hr at 20° C. while the reduced antibody was conjugated to BI-P203 payload via the maleimide group. After conjugation was completed, the reaction mixture was desalted and concentrated to yield anti-Trop-2-DM1 ADCs. The biochemical properties of the resulting ADCs were characterized using size-exclusion chromatography high pressure liquid chromatography (SEC-HPLC) to determine purity and aggregation content, and by using hydrophobic interaction chromatography HPLC (HIC-HPLC) to confirm drug loading (DAR). The conjugation procedure is illustrated in FIG. 12. The final ADC products are comprised of four or six or seven DM1-linker molecules. The cysteine conjugation method used in the conjugation process produces more homogenous ADCs compared to lysine conjugation method.

### Example 9

### In Vitro Cytotoxicity of Anti-Trop-2 ADCs in Cancer Cell Lines Different Trop-2 Expression Levels

Anti-Trop-2 ADCs were conjugated with different DM1 derivatives with similar DARs, and were tested ahead to ahead with anti-Trop-2-SMCC-DM1 and anti-Trop-2-vc-MMAE against BxPC-3 pancreatic cancer cells representing high Trop-2 expression, MDA-MB-468 breast cancer cells, N87 gastric cancer cells, and SK-BR-3 ovarian cancer cells representing moderate Trop-2 expression, Colo205 colon cancer cells representing low Trop-2 expression, and A549 lung cancer cells and MDA-MB-231 breast cancer cells with undetectable level of Trop-2. In vitro cytotoxicity assay was performed. Briefly, all cell lines were cultured in a suitable culture medium at 37° C. in a humidified incubator atmosphere of 5% CO2. Cells were plated in 96-well flat bottom plates. Cell seeding number ranged from 500 cells//100 ul/well to 6,000 cells/100 μl/well. Cells were allowed to adhere overnight at 37° C. in a humidified atmosphere of 5% CO2. ADCs were prepared from stock solution and diluted into appropriated working concentration 24 hours after cell seeding. A serial ten-fold dilution for seven points was performed with culture medium. The final concentrations were ranging from 10,000 nM to 0.001 nM. The cells were incubated with ADCs for 72 hours. Cell Counting Kit-8 solution (Dojindo China Co., Ltd, lot #PL701) was added to the wells for 1-4 hours at 37° C. and the absorbance at 450 nm was measured using a Microplate Reader (SpectraMax M5, Molecular Devices) and SoftMax Pro5.4.1 software. Dose-response curves were generated and IC50 was calculated using GraphPad Prism 7 three-parameter curve fitting.

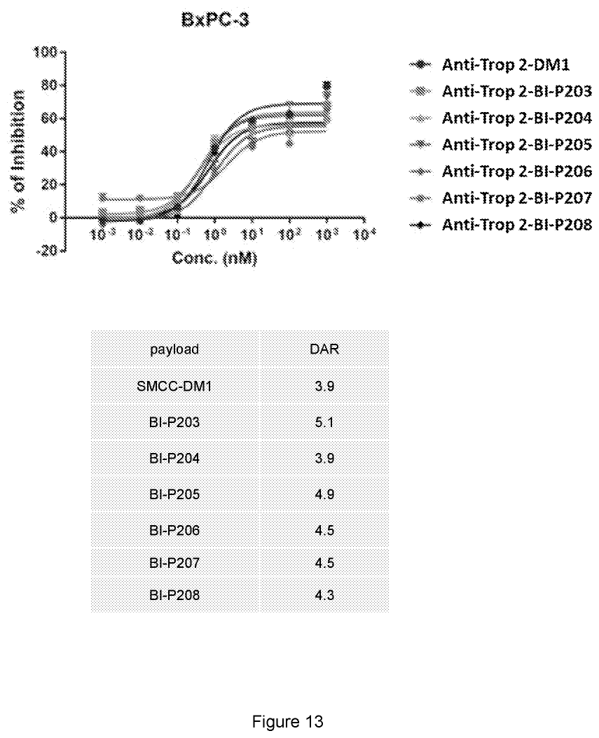

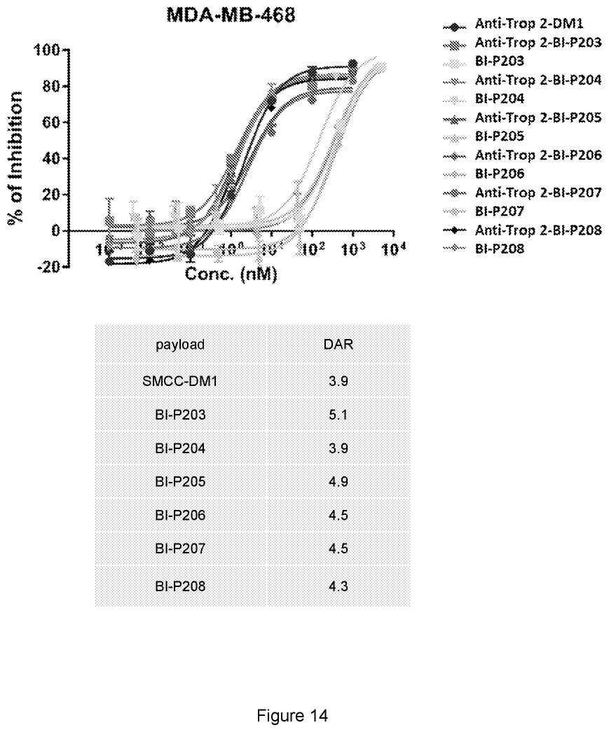

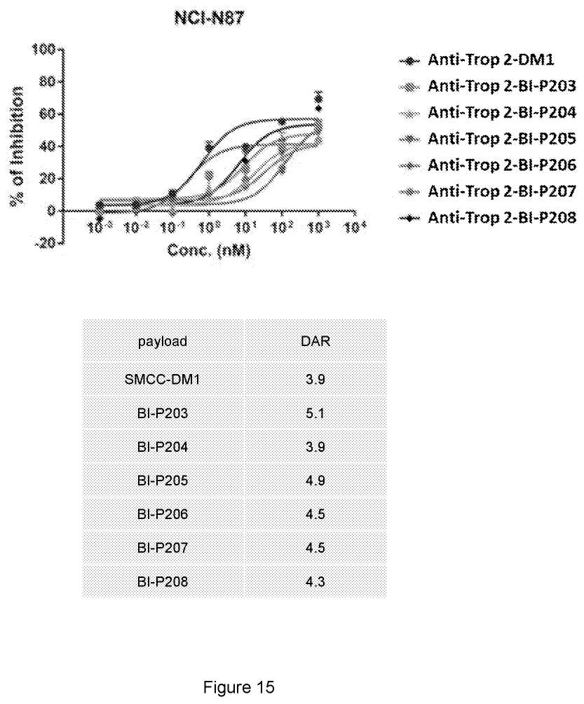

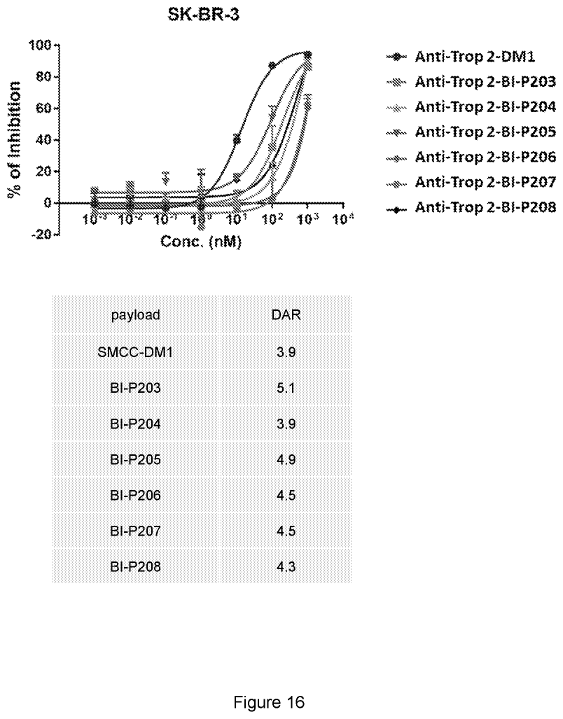

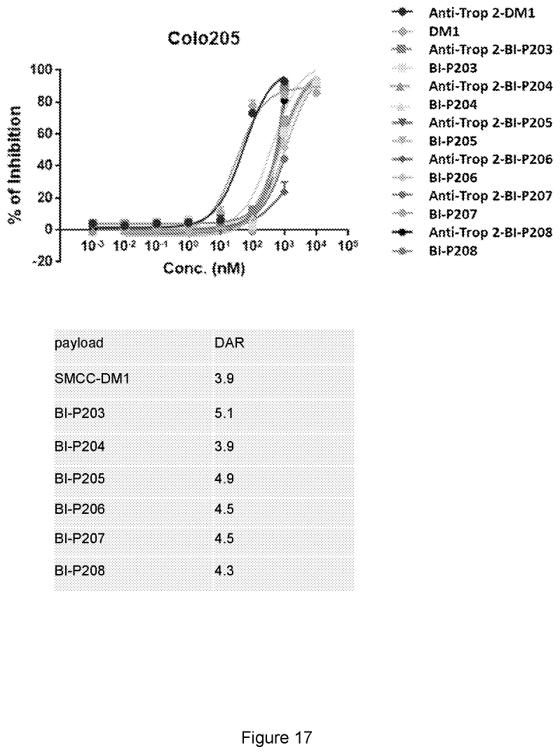

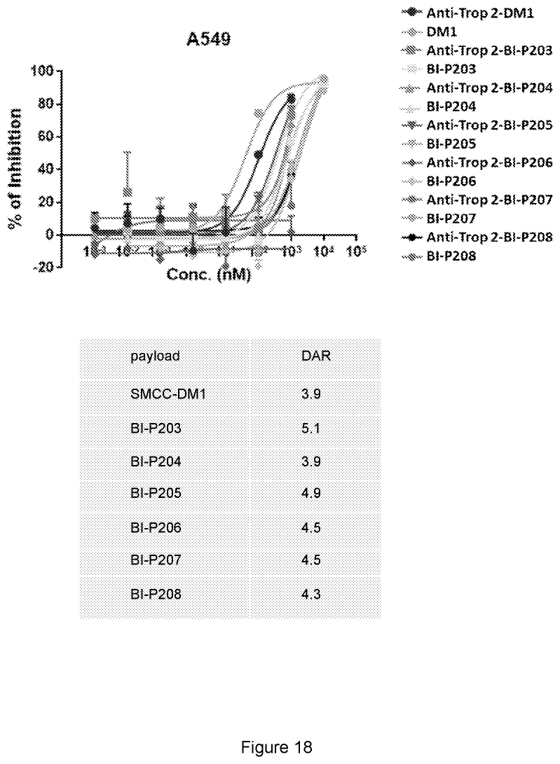

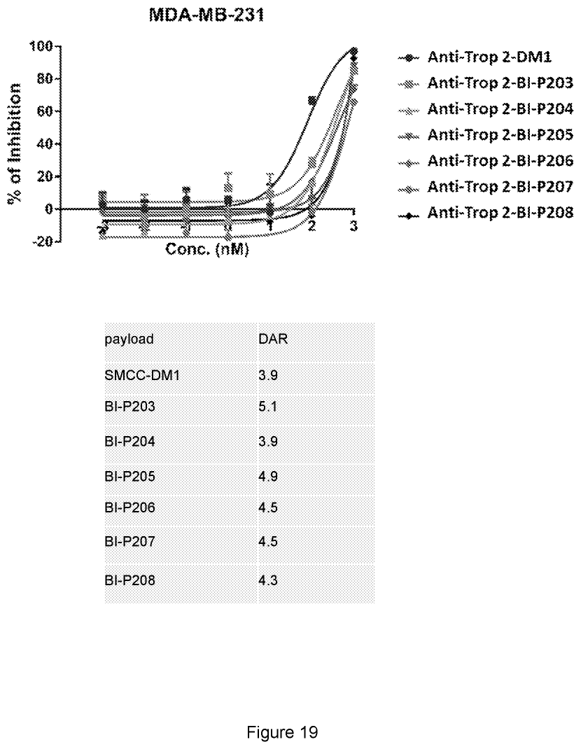

FIG. 13-FIG. 19 show the representative killing curves of ADCs containing payload SMCC-DM1, BI-P203, BI-P204, BI-P205, BI-P206, BI-P207, BI-P208 in BxPC-3 (FIG. 13), MDA-MB-468 (FIG. 14), N87 (FIG. 15), SK-BR-3 (FIG. 16), Colo205 (FIG. 17), A549 (FIG. 18), and MDA-MB-231 (FIG. 19) cells. Overall, all ADCs containing various DM1 derivative payloads demonstrated similar in vitro killing activities as ADC-DM1 in Trop-2 high or medium cells. They were less potent than ADC-DM1 against cells that had low or no Trop-2 expression.

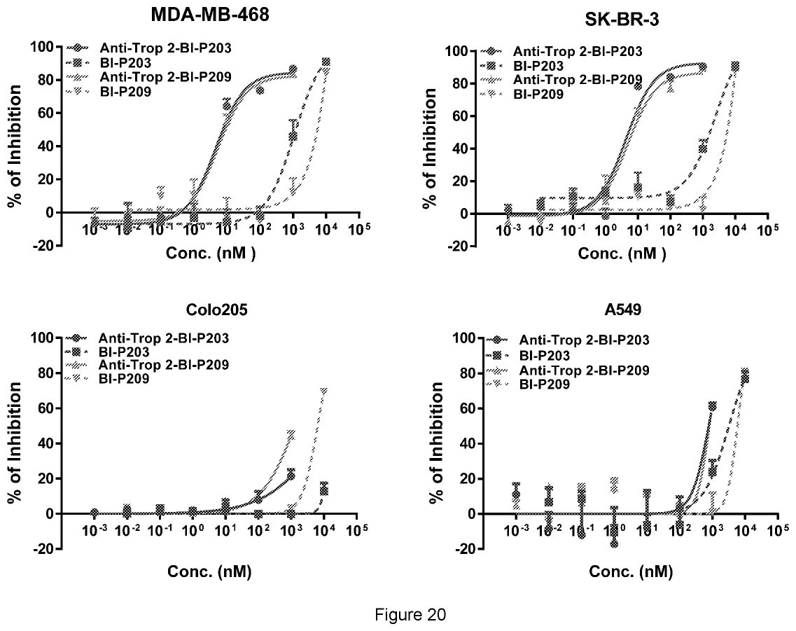

ADC-BI-P209 and free payload together with ADC-BI-P203 and its free payload were tested separately in MDA-MB-468, SK-BR-3, Colo205, and A549 cells. As shown in FIG. 20, ADC-BI-P209 showed comparable activity as ADC-BI-P203, while free BI-P209 was slightly more potent than free BI-P203 in all tested cells.

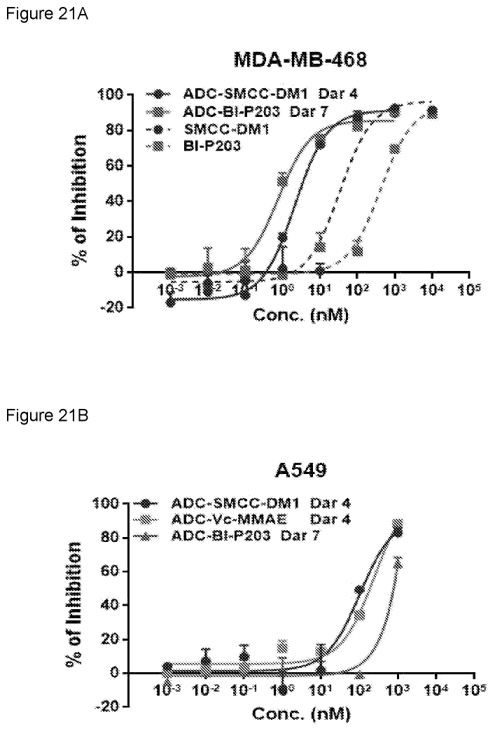

ADC-BI-P203 was also conjugated at an antibody to drug ratio of 7 (DAR=7), and its in vitro activity was compared with ADC-SMCC-DM1 which had DAR of 4 in Trop-2 positive MDA-M B-468 and Trop-2 negative A549 cells. As shown in FIG. 21, ADC-BI-P203 was more potent against Trop-2 high expresser MDA-MB-468 cells than ADC-SMCC-DM1, while the free BI-P203 payload had less activity than that of free SCMM-DM1 payload. In Trop-2 negative A549 cells, ADC-BI-P203 was the weakest among all 3 ADCs tested.

Table 3 summarizes the results from in vitro cytotoxicity assays in which ADCs containing various DM1 derivative payload at different DARs were tested. Results demonstrate that the ADC-BI-P203 with DAR7 had comparable cell killing activity to ADC-vc-MMAE (DAR4), and both ADCs were more potent than ADC-SMCC-DM1(DAR4) in those tumor cells that have high/moderate Trop-2 expression (i.e., BxPC-3, MDA-MB-468, NCI-N87). On the other hand, in the tumor cells express low Trop-2 levels (Colo-205), the in vitro cytotoxicity of ADC-BI-P203 was similar to that of ADC-SMCC-DM1, but less potent than ADC-vc-MMAE. In the Trop-2 negative A549 and MDA-MB-231 cells, IC50 of ADC-SMCC-DM1 and ADC-vc-MMAE was around 100 nM, while 1050 could not be calculated with ADC—BI-P203 (>500 nM), indicating no specific killing activity was achieved. Taken together, these in vitro cytoxicity results suggest that the BI-P203 containing ADCs remain the same potent anti-tumor activity as MMAE ADCs or DM1 ADCs particularly against high antigen expressing tumors, while having less effect on low or no antigen expressing cells i.e., normal cells, therefore provide a wider therapeutic window.

TABLE 3

| | | IC50 (nM) | | | | | | | | | |
|---|---|---|---|---|---|---|---|---|---|---|---|
| | | Anti-Trop | | | Anti-Trop | | Anti-Trop | | | Anti-Trop | |
| | | 2-BI-P203 | | | 2-BI-P204 | | 2-BI-P205 | | | 2-BI-P206 | |
| Cell | TROP2 | DAR | DAR | DAR | DAR | DAR | DAR | DAR | DAR | DAR | DAR |
| line | Expression | 3.5 | 5.1 | 7 | 2.8 | 3.9 | 3.6 | 4.9 | 7.1 | 3.6 | 4.5 |
| BxPC-3 | +++ | 0.48 | | 0.59 | 0.38 | | 0.43 | | 0.52 | 0.99 | |
| MDA-MB-468 | ++ | | 1.20 | 0.77 | | 1.81 | | 1.58 | 0.47 | | 2.663 |
| NCI-N87 | ++/+ | 8.93 | | 0.62 | 17.56 | | 0.282 | | | 124.6 | |
| Colo205 | + | | | 31.75 | | | | | 26.09 | | |
| A549 | − | | | >500 | | | >100 | | | | |
| MDA-MB-231 | − | | >500 | >500 | | >500 | | | | | >500 |

| | IC50 (nM) | | | | | | |
|---|---|---|---|---|---|---|---|
| | Anti-Trop | | Anti-Trop | | Anti-Trop | Anti-Trop | Anti-Trop |
| | 2-BI-P207 | | 2-BI-P208 | | 2-BI-P209 | 2-SMCC-DM1 | 2-Vc-MMAE |
| Cell | DAR | DAR | DAR | DAR | DAR | DAR | DAR |
| line | 3.4 | 4.5 | 3.0 | 4.3 | 6.2 | 4 | 4 |
| BxPC-3 | 2.091 | | 0.67 | | | 1.01 | 0.51 |
| MDA-MB-468 | | 4.29 | | 2.224 | 5.366 | 4.88 | 0.54 |
| NCI-N87 | 33.75 | | 6.432 | | | 2.88 | 0.49 |
| Colo205 | | | | | >500 | 56.42 | 0.68 |
| A549 | | | | | >500 | >100 | >100 |
| MDA-MB-231 | | >500 | | | | >100 | >100 |

### Example 10

### In Vivo Characterization of Anti-Trop-2-BI-P203

The anti-tumor activities of anti-Trop-2-BI-P203 ADC was assessed using Trop-2-positive MDA-MB-468 and Trop-2-negative Colo205 xenograft models in mouse subjects. Five million cells were harvested from culture flasks and implanted subcutaneously into the right flank of 6- to 7-week-old BALB/c nude mice. Dosing started when tumors were established. Anti-Trop-2-BI-P203 was administered at 1.5 and 5.0 mg/kg single dose. Tumors were measured twice a week throughout the course of the experiments, with tumor volume calculated using the following formula: tumor volume (mm³)=(length×width²)/2.

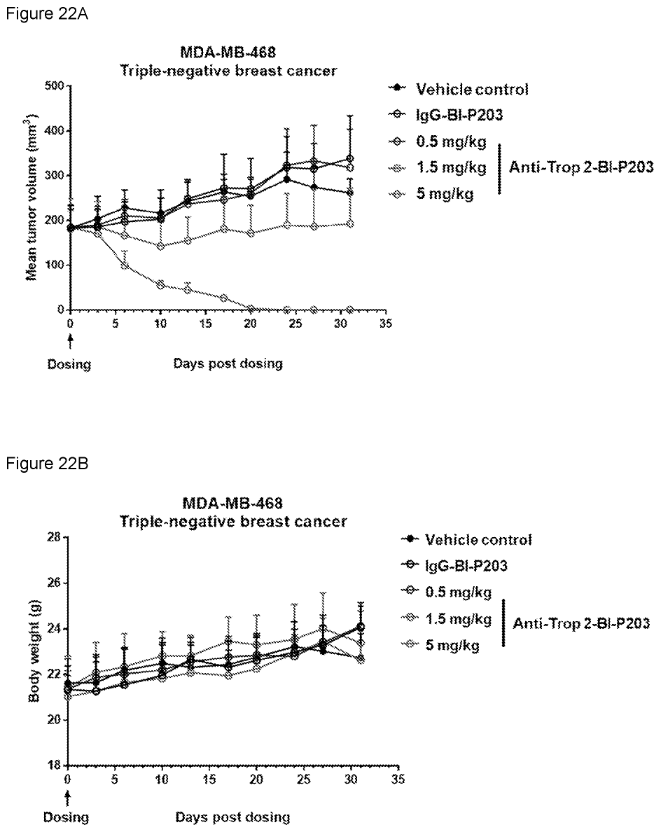

FIG. 22A shows the results of the in vivo MDA-MB-468 xenograft study. As shown, single administration of ADC at 5.0 mg/kg induced complete tumor regression. The 1.5 mg/kg dose of ADC resulted in stable disease. Anti-Trop-2-BI-P203 had no obvious effect on mouse body weigh change compared to vehicle control or IgG control ADC as show in FIG. 22B.

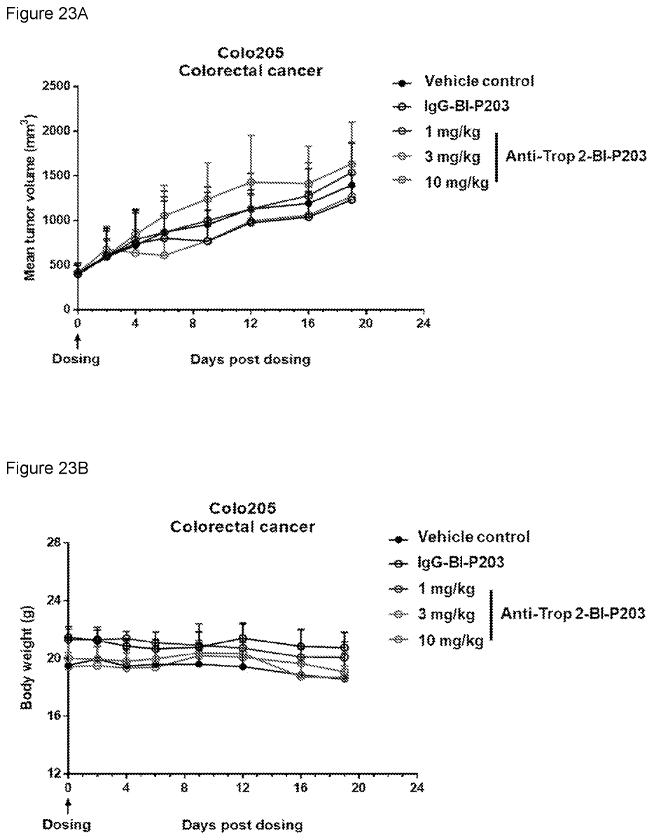

FIG. 23 depicts the results of the in vivo Colo205 xenograft study. The result indicates that anti-Trop-2-BI-P203 was not effective at all tested doses of 1, 3, and 10 mg/kg in this Trop-2-negative tumor model (FIG. 23A) and did affect mouse body weight change (FIG. 23B).

All of the articles and methods disclosed and claimed herein can be made and executed without undue experimentation in light of the present invention. While the articles and methods of this invention have been described in terms of preferred embodiments, it will be apparent to those of skill in the art that variations may be applied to the articles and methods without departing from the spirit and scope of the invention. All such variations and equivalents apparent to those skilled in the art, whether now existing or later developed, are deemed to be within the spirit and scope of the invention as defined by the appended claims. All patents, patent applications, and publications mentioned in the specification are indicative of the levels of those of ordinary skill in the art to which the invention pertains. All patents, patent applications, and publications are herein incorporated by reference in their entirety for all purposes and to the same extent as if each individual publication was specifically and individually indicated to be incorporated by reference in its entirety for any and all purposes. The invention illustratively described herein suitably may be practiced in the absence of any element(s) not specifically disclosed herein. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

### SEQUENCE LISTINGS

The nucleic and amino acid sequences listed in the accompanying sequence listing are shown using standard letter abbreviations for nucleotide bases and three letter code for amino acids, as defined in 37 C.F.R. 1.822.

SEQ ID NO: 1 is an amino acid sequence comprising a light chain of anti-Trop-2 antibody.

SEQ ID NO: 2 is an amino acid sequence comprising a heavy chain of anti-Trop-2 antibody.

SEQ ID NO: 3 is an amino acid sequence of a light chain CDR1 in anti-Trop-2 antibody.

SEQ ID NO: 4 is an amino acid sequence of a light chain CDR2 in anti-Trop-2 antibody.

SEQ ID NO: 5 is an amino acid sequence of a light chain CDR3 in anti-Trop-2 antibody.

SEQ ID NO: 6 is an amino acid sequence of a heavy chain CDR1 in anti-Trop-2 antibody.

SEQ ID NO: 7 is an amino acid sequence of a heavy chain CDR2 in anti-Trop-2 antibody.

SEQ ID NO: 8 is an amino acid sequence of a heavy chain CDR3 in anti-Trop-2 antibody.

SEQUENCE LISTINGS
Anti-Trop-2 antibody light chain
amino acid sequence
SEQ ID NO: 1
DIVMTQSPDSLAVSLGERATINCKSSQSLLNSGTR
KNYLAWYQQKPGQPPKLLISWASSRESGVPDRFSG
SGSGTDFTLTISSLQAEDVAVYYCKQSYNLFTFGG
GTKVEIKRTVAAPSVFIFPPSDEQLKSGTASVVCL
LNNFYPREAKVQWKVDNALQSGNSQESVTEQDSKD
STYSLSSTLTLSKADYEKHKVYACEVTHQGLSSPV
TKSFNRGEC*
Anti-Trop-2 antibody heavy chain
amino acid sequence
SEQ ID NO: 2
QVQLQESGPGLVKPSETLSLTCTVSGFSLTSYGVN
WIRQPPGKGLEWIGVMWAGGSTNYNSALMSRLTIS
KDTSKNQFSLKLSSVTAADTAVYYCARDENWDGAW
FAYWGQGTLVTVSSASTKGPSVFPLAPSSKSTSGG
TAALGCLVKDYFPEPVTVSWNSGALTSGVHTFPAV
LQSSGLYSLSSVVTVPSSSLGTQAYICNVNHKPSN
TKVDKKVGPKSCDKTHTCPPCPAPELLGGPSVFLF
PPKPKDTLMISRTPEVTCVVVDVSHEDPEVKFNWY
VDGVEVHNAKTKPREEQYNSTYRVVSVLTVLHQDW
LNGKEYKCKVSNKALPAPIERTISKAKGQPREPQV
YTLPPSRDELAKNQVSLTCLVKGFYPSDIAVEWES
NGQPENNYKTTPPVLDSDGSFFLYSKLTVDKSRWQ
QGNVFSCSVMHEALHNHYTQKSLSLSPGK*
Anti-Trop-2 antibody light chain
CDR1 amino acid sequence
SEQ ID NO: 3
KSSQSLLNSGTRKNYLA
Anti-Trop-2 antibody light chain
SEQ ID NO: 4
CDR2 amino acid sequence
WASSRES
Anti-Trop-2 antibody light chain
SEQ ID NO: 5
CDR3 amino acid sequence
KQSYNLFT
Anti-Trop-2 antibody heavy chain
CDR1 amino acid sequence
SEQ ID NO: 6
SYGVN
Anti-Trop-2 antibody heavy chain
CDR2 amino acid sequence
SEQ ID NO: 7
VMWAGGSTNYNSALMS
Anti-Trop-2 antibody heavy chain
CDR3 amino acid sequence
SEQ ID NO: 8
DENWDGAWFAY

1. An antibody drug conjugate (ADC) comprising an antibody chemically linked to a derivatized maytansinol or maytansinol analog residue represented by the following formula (I):
[MayO-L-]ₓ-Ab (I)
wherein x is about 1 to about 10;
Ab is an antibody or antigen binding fragment thereof;
wherein MayO is a maytansinol or maytansinol analog;
L is a bivalent linker comprising a N-methylalanine moiety represented by the following formula: 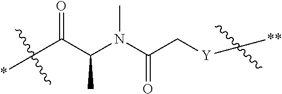
wherein * indicates the point of attachment to MayO, ** indicates the point of attachment to Ab;
Y is selected from 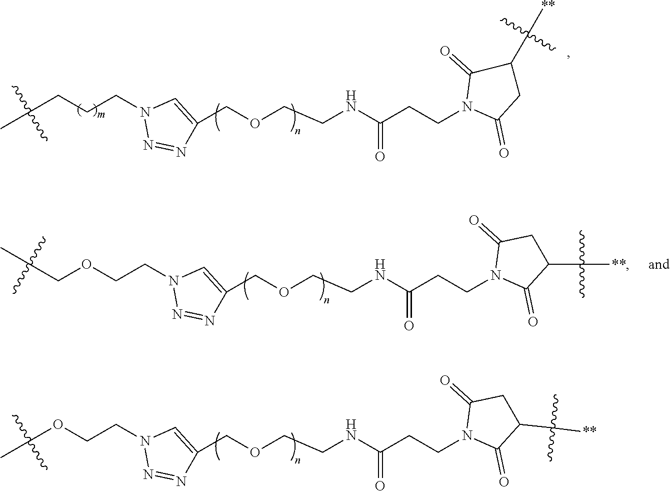
wherein m is 0-8, and n=2-12.

2. The ADC having the formula I of claim 1, wherein Ab is capable of binding to a tumor associated antigen (TAA) selected from the group consisting of Trop-2, Her2, Her3, Her4, EGF, EGFR, CD2, CD3, CD5, CD7, CD13, CD19, CD20, CD21, CD23, CD30, CD33, CD34, CD38, CD46, CD55, CD59, CD69, CD70, CD71, CD97, CD117, CD123, CD127, CD134, CD137, CD138, CD146, CD147, CD152, CD154, CD174, CD195, CD200, CD205, CD212, CD223, CD227, CD253, CD272, CD274, CD276, CD278, CD279, CD309, CD319, CD326, CD340, DR6, Kv1.3, 5E10, MUC1, uPA, MAGE3, MUC16, KLK3, K-ras, Mesothelin, p53, Survivin, G250, PSMA, Endoplasmin, BCMA, GPNMB, EphA2, EphB2, TMEFF2, Integrin beta 6, 5T4, CA9, IGF-1R, Axl, B7H3, B7H4, CDH6, HAVCR1, STEAP-1, STEAP-2, UPK2, and CLDN18.

3. The ADC having the formula I of claim 1, wherein Ab is an anti-Trop-2 antibody or a binding fragment thereof.

4. A derivatized maytansinol or maytansinol analogs represented by the following formula (II):
MayO-L′ (II)
wherein MayO is maytansinol or maytansinol analogs, L′ is a bivalent linker comprising a N-methylalanine moiety represented by the following formula: 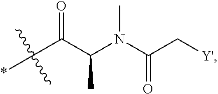
wherein * indicates the point of attachment to MayO; and Y′ comprises a functional group which can attach to an antibody.

5. The maytansinol or maytansinol analog of formula II of claim 4, wherein Y′ comprises pyrroline-dione.

6. The maytansinol or maytansinol analog of formula II of claim 4, wherein Y′ is selected from 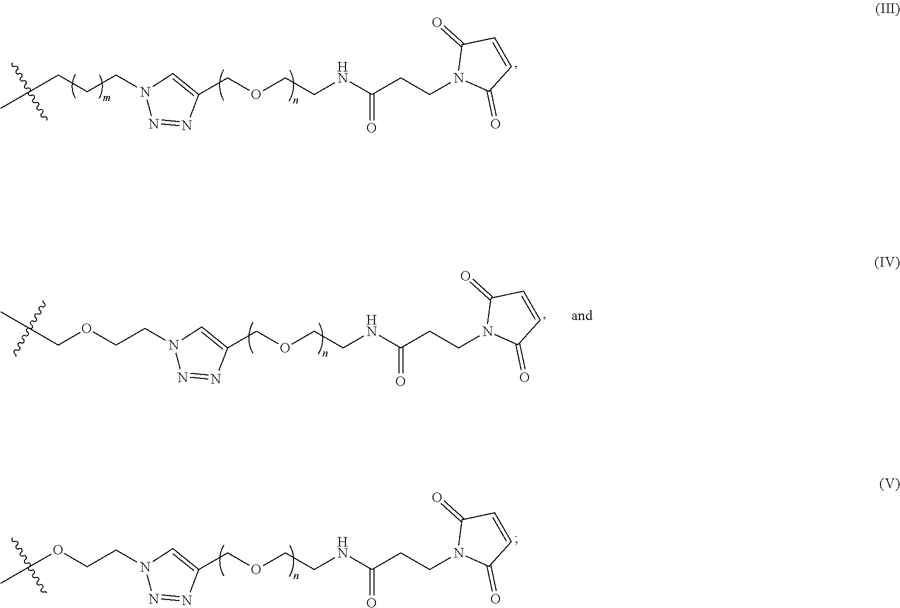
m is 0 to 8; and n is 2 to 12.

7. A derivatized maytansinol or maytansinol analog residue represented by the following formula (VI):
MayO-L²′ (VI)
wherein MayO is maytansinol or a maytansinol analog, L²′ is a bivalent linker represented by the following formula: *—C(═O)R—Y″, wherein
* indicates the point of attachment to MayO;
R is a 3-7 membered heterocyclyl, aryl or cyclic alkyl ring; and
Y″ comprises a functional group which can attach to an antibody.

8. An antibody drug conjugate (ADC) comprising an antibody chemically linked to the derivatized maytansinol or maytansinol analog residue of claim 7, wherein the ADC is represented by the following formula (VII):
[MayO-L²-]ₓ-Ab (VII)
wherein x is about 1 to about 10;
Ab is an antibody or antigen binding fragment thereof;
wherein MayO is maytansinol or a maytansinol analog;
L² is a bivalent linker represented by the following formula: *—C(═O)R—Y″—**, wherein
* indicates the point of attachment to MayO, ** indicates the point of attachment to Ab;
R is a 3-7 membered heterocyclyl, aryl or cyclic alkyl ring; and
Y″ comprises a functional group which can attach to an antibody.

9. The ADC of formula VII of claim 8, wherein Ab is capable of binding to a tumor associated antigen (TAA) selected from the group consisting of Trop-2, Her2, Her3, Her4, EGF, EGFR, CD2, CD3, CD5, CD7, CD13, CD19, CD20, CD21, CD23, CD30, CD33, CD34, CD38, CD46, CD55, CD59, CD69, CD70, CD71, CD97, CD117, CD123, CD127, CD134, CD137, CD138, CD146, CD147, CD152, CD154, CD174, CD195, CD200, CD205, CD212, CD223, CD227, CD253, CD272, CD274, CD276, CD278, CD279, CD309, CD319, CD326, CD340, DR6, Kv1.3, 5E10, MUC1, uPA, MAGE3, MUC16, KLK3, K-ras, Mesothelin, p53, Survivin, G250, PSMA, Endoplasmin, BCMA, GPNMB, EphA2, EphB2, TMEFF2, Integrin beta 6, 5T4, CA9, IGF-1R, Axl, B7H3, B7H4, CDH6, HAVCR1, STEAP-1, STEAP-2, UPK2, and CLDN18.

10. An ADC of formula VII of claim 8, wherein Ab is an anti-Trop-2 antibody or a binding fragment thereof.

11. The ADC of formula VII of claim 8, wherein L² is a bivalent linker selected from: 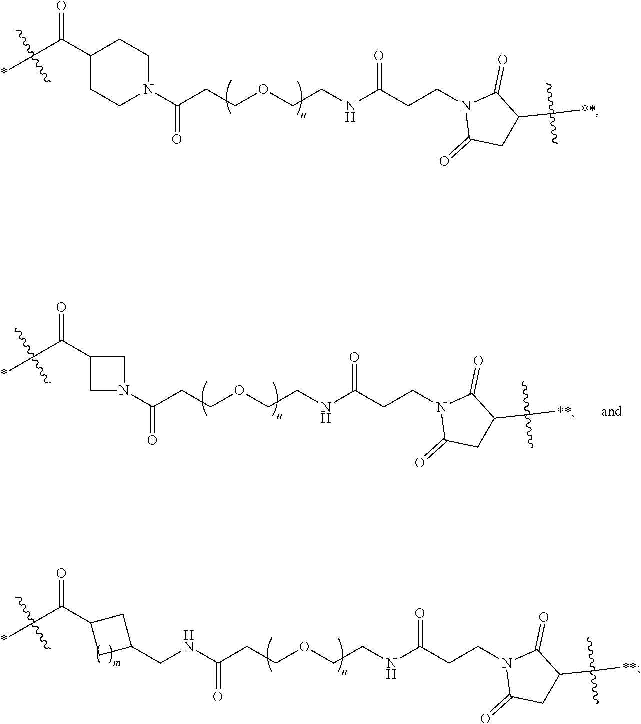
wherein m=0-3; and n=2-12.

12. The ADC of formula VII of claim 8, wherein L² comprises pyrroline-dione.

13. The maytansinol or maytansinol analog of formula VI of claim 7, wherein L²′ is a linker having the formula of (VIII): 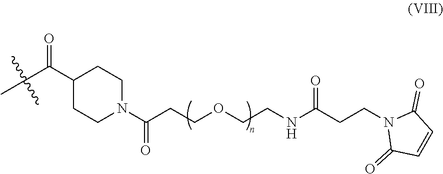
wherein n=2-12.

14. The maytansinol or maytansinol analog of formula VI of claim 7, wherein L²′ is a linker having the formula of (IX): 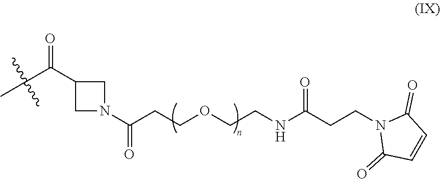
wherein n=2-12.

15. The maytansinol or maytansinol analog of formula VI of claim 7, wherein L²′ is a linker having the formula of (X):

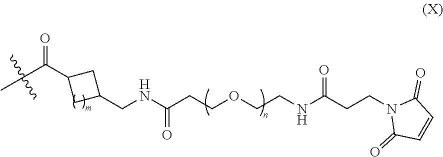

16. The ADC of formula VII of claim 8, L² is a non-cleavable linker.

17. The ADC of formula VII of claim 8, wherein the heterocyclyl ring is selected from saturated or unsaturated 4-6 membered nitrogen containing heterocyclic rings.

18. A pharmaceutical composition comprising an ADC of claim 1.

19. A method for treating a cancer comprising administering to a subject in need thereof a pharmaceutical composition according to claim 18.

20. (canceled)

21. A method according to claim 19, wherein the cancer is selected from the group consisting of pancreatic cancer, gastric cancer, liver cancer, breast cancer, ovarian cancer, colorectal cancer, melanoma, leukemia, myelodysplastic syndrome, lung cancer, prostate cancer, brain cancer, bladder cancer, head-neck cancer, and rhabdomyosarcoma.

22-24. (canceled)
